SEQUENCE LISTING (242 sequences)

SEQ ID NO: 1 - protein, 5 aa, synthetic construct
SYSMN

SEQ ID NO: 2 - protein, 17 aa, synthetic construct
SISSSSSYIYYADSVKG

SEQ ID NO: 3 - protein, 12 aa, synthetic construct
RHGYSNSDAFDN

SEQ ID NO: 4 - protein, 11 aa, synthetic construct
RASQGISHYLV

SEQ ID NO: 5 - protein, 7 aa, synthetic construct
AASSLQS

SEQ ID NO: 6 - protein, 9 aa, synthetic construct
LQHNSYPWT

SEQ ID NO: 7 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 8 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 9 - protein, 223 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKC

SEQ ID NO: 10 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 381 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 12 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQCGNSQESVTEQDSKDSTYSLSSTLTLSKADYEKHKVYACEVTHQGLSSPVTKSFNRGEC

SEQ ID NO: 13 - protein, 447 aa, synthetic construct
  VARIANT 128: note = X is S or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDICVEWESNGQPENNYKTTPPVLDSDGSFFLYSVLTVDKSRWQEGNVFSCSVMHEALHNHYTQKSLSLSLG

SEQ ID NO: 15 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDICVEWESNGQPENNYKTTPPVLDSDGSFFLASRLTVDKSRWQEGNVFSCSVMHEALHNHYTQKSLSLSLG

SEQ ID NO: 16 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 17 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 18 - protein, 446 aa, synthetic construct
  VARIANT 127: note = X is S or C
QVQLVQSGAEVKKPGSSVKVSCKASGYTFSSYAIEWVRQAPGQGLEWMGGILPGSGTINYNEKFKGRVTITADKSTSTAYMELSSLRSEDTAVYYCARMSSNSDQGFDLWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 19 - protein, 214 aa, synthetic construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 5 aa, synthetic construct
ETAVA

SEQ ID NO: 21 - protein, 17 aa, synthetic construct
GIGGGVDITYYADSVKG

SEQ ID NO: 22 - protein, 17 aa, synthetic construct
RPGRPLITSKVADLYPY

SEQ ID NO: 23 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 24 - protein, 20 aa, synthetic construct
GGGGQGGGGQGGGGQGGGGQ

SEQ ID NO: 25 - protein, 6 aa, synthetic construct
GSYWIC

SEQ ID NO: 26 - protein, 18 aa, synthetic construct
CIYSTSGGRTYYASWVKG

SEQ ID NO: 27 - protein, 12 aa, synthetic construct
GDDSISDAYFDL

SEQ ID NO: 28 - protein, 13 aa, synthetic construct
QSSQSVYNNNRLA

SEQ ID NO: 29 - protein, 7 aa, synthetic construct
DASTLAS

SEQ ID NO: 30 - protein, 12 aa, synthetic construct
QGTYFSSGWSWA

SEQ ID NO: 31 - protein, 121 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSS

SEQ ID NO: 32 - protein, 112 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVK

SEQ ID NO: 33 - protein, 447 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 219 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - RNA, 21 nt, synthetic construct
  source 1..21: mol_type = other RNA
agcaaaaccattgcttcacta

SEQ ID NO: 36 - RNA, 23 nt, synthetic construct
  source 1..23: mol_type = other RNA
tagtgaagcaatggttttgctgt

SEQ ID NO: 37 - RNA, 21 nt, synthetic construct
  source 1..21: mol_type = other RNA
cagcaaaaccattgcttcaca

SEQ ID NO: 38 - RNA, 23 nt, synthetic construct
  source 1..23: mol_type = other RNA
tgtgaagcaatggttttgctgtc

SEQ ID NO: 39 - RNA, 21 nt, synthetic construct
  source 1..21: mol_type = other RNA
gatgcctgaacttgaattaaa

SEQ ID NO: 40 - RNA, 23 nt, synthetic construct
  source 1..23: mol_type = other RNA
tttaattcaagttcaggcatcta

SEQ ID NO: 41 - RNA, 21 nt, synthetic construct
  source 1..21: mol_type = other RNA
ctgtaacacaagtagatgcca

SEQ ID NO: 42 - RNA, 23 nt, synthetic construct
  source 1..23: mol_type = other RNA
tggcatctacttgtgttacagca

SEQ ID NO: 43 - RNA, 21 nt, synthetic construct
  source 1..21: mol_type = other RNA
cctgatcactatgcattttaa

SEQ ID NO: 44 - RNA, 23 nt, synthetic construct
  source 1..23: mol_type = other RNA
ttaaaatgcatagtgatcaggaa

SEQ ID NO: 45 - RNA, 21 nt, synthetic construct
  source 1..21: mol_type = other RNA
tgacagctgtgctgtaacaca

SEQ ID NO: 46 - RNA, 23 nt, synthetic construct
  source 1..23: mol_type = other RNA
tgtgttacagcacagctgtcaaa

SEQ ID NO: 47 - RNA, 21 nt, synthetic construct
  source 1..21: mol_type = other RNA
catgaatagattctctcctga

SEQ ID NO: 48 - RNA, 23 nt, synthetic construct
  source 1..23: mol_type = other RNA
tcaggagagaatctattcatgca

SEQ ID NO: 49 - RNA, 21 nt, synthetic construct
  source 1..21: mol_type = other RNA
gtacagaatcattgcttatga

SEQ ID NO: 50 - RNA, 23 nt, synthetic construct
  source 1..23: mol_type = other RNA
tcataagcaatgattctgtacaa

SEQ ID NO: 51 - RNA, 21 nt, synthetic construct
  source 1..21: mol_type = other RNA
tgtccacgtatctttgggtca

SEQ ID NO: 52 - RNA, 23 nt, synthetic construct
  source 1..23: mol_type = other RNA
tgacccaaagatacgtggacaaa

SEQ ID NO: 53 - RNA, 21 nt, synthetic construct
  source 1..21: mol_type = other RNA
gtccacgtatctttgggtcta

SEQ ID NO: 54 - RNA, 23 nt, synthetic construct
  source 1..23: mol_type = other RNA
tagacccaaagatacgtggacaa

SEQ ID NO: 55 - RNA, 21 nt, synthetic construct
  source 1..21: mol_type = other RNA
gatataggaattaagaggata

SEQ ID NO: 56 - RNA, 23 nt, synthetic construct
  source 1..23: mol_type = other RNA
tatcctcttaattcctatatcac

SEQ ID NO: 57 - RNA, 21 nt, synthetic construct
  source 1..21: mol_type = other RNA
acagattgctgcttctgctaa

SEQ ID NO: 58 - RNA, 23 nt, synthetic construct
  source 1..23: mol_type = other RNA
ttagcagaagcagcaatctgtac

SEQ ID NO: 59 - RNA, 21 nt, synthetic construct
  source 1..21: mol_type = other RNA
ccaagatgcagcagaacggca

SEQ ID NO: 60 - RNA, 23 nt, synthetic construct
  source 1..23: mol_type = other RNA
tgccgttctgctgcatcttggac

SEQ ID NO: 61 - RNA, 21 nt, synthetic construct
  source 1..21: mol_type = other RNA
aggaagcagccaacgagagaa

SEQ ID NO: 62 - RNA, 23 nt, synthetic construct
  source 1..23: mol_type = other RNA
ttctctcgttggctgcttcctgt

SEQ ID NO: 63 - RNA, 21 nt, synthetic construct
  source 1..21: mol_type = other RNA
ctttgagcagatgcagaacta

SEQ ID NO: 64 - RNA, 23 nt, synthetic construct
  source 1..23: mol_type = other RNA
tagttctgcatctgctcaaagaa

SEQ ID NO: 65 - RNA, 21 nt, synthetic construct
  source 1..21: mol_type = other RNA
ggcagttatccagcatttcca

SEQ ID NO: 66 - RNA, 23 nt, synthetic construct
  source 1..23: mol_type = other RNA
tggaaatgctggataactgcctt

SEQ ID NO: 67 - RNA, 21 nt, synthetic construct
  source 1..21: mol_type = other RNA
tccaacctacaagttctttga

SEQ ID NO: 68 - RNA, 23 nt, synthetic construct
  source 1..23: mol_type = other RNA
tcaaagaacttgtaggttggatt

SEQ ID NO: 69 - RNA, 21 nt, synthetic construct
  source 1..21: mol_type = other RNA
ccaacctacaagttctttgaa

SEQ ID NO: 70 - RNA, 23 nt, synthetic construct
  source 1..23: mol_type = other RNA
ttcaaagaacttgtaggttggat

SEQ ID NO: 71 - RNA, 21 nt, synthetic construct
  source 1..21: mol_type = other RNA
ctgaagaagaaacagtacaca

SEQ ID NO: 72 - RNA, 23 nt, synthetic construct
  source 1..23: mol_type = other RNA
tgtgtactgtttcttcttcagca

SEQ ID NO: 73 - RNA, 21 nt, synthetic construct
  source 1..21: mol_type = other RNA
gacaaagtagtagaagtagca

SEQ ID NO: 74 - RNA, 23 nt, synthetic construct
  source 1..23: mol_type = other RNA
tgctacttctactactttgtctt

SEQ ID NO: 75 - RNA, 21 nt, synthetic construct
  source 1..21: mol_type = other RNA
aagttctttgagcagatgcaa

SEQ ID NO: 76 - RNA, 23 nt, synthetic construct
  source 1..23: mol_type = other RNA
ttgcatctgctcaaagaacttgt

SEQ ID NO: 77 - RNA, 21 nt, synthetic construct
  source 1..21: mol_type = other RNA
aggcagttatccagcatttca

SEQ ID NO: 78 - RNA, 23 nt, synthetic construct
  source 1..23: mol_type = other RNA
tgaaatgctggataactgccttc

SEQ ID NO: 79 - RNA, 21 nt, synthetic construct
  source 1..21: mol_type = other RNA
agcaccgagagagaatgtcca

SEQ ID NO: 80 - RNA, 23 nt, synthetic construct
  source 1..23: mol_type = other RNA
tggacattctctctcggtgcttg

SEQ ID NO: 81 - RNA, 21 nt, synthetic construct
  source 1..21: mol_type = other RNA
ccggtcccaggttatgacaca

SEQ ID NO: 82 - RNA, 23 nt, synthetic construct
  source 1..23: mol_type = other RNA
tgtgtcataacctgggaccggat

SEQ ID NO: 83 - RNA, 21 nt, synthetic construct
  source 1..21: mol_type = other RNA
aaccagtgaccatccagaaca

SEQ ID NO: 84 - RNA, 23 nt, synthetic construct
  source 1..23: mol_type = other RNA
tgttctggatggtcactggttgg

SEQ ID NO: 85 - RNA, 21 nt, synthetic construct
  source 1..21: mol_type = other RNA
accgaggactgaccactcgaa

SEQ ID NO: 86 - RNA, 23 nt, synthetic construct
  source 1..23: mol_type = other RNA
ttcgagtggtcagtcctcggtcg

SEQ ID NO: 87 - RNA, 21 nt, synthetic construct
  source 1..21: mol_type = other RNA
cacaccgtcgccaaagagaca

SEQ ID NO: 88 - RNA, 23 nt, synthetic construct
  source 1..23: mol_type = other RNA
tgtctctttggcgacggtgtgcc

SEQ ID NO: 89 - RNA, 21 nt, synthetic construct
  source 1..21: mol_type = other RNA
gccaagcaccgagagagaata

SEQ ID NO: 90 - RNA, 23 nt, synthetic construct
  source 1..23: mol_type = other RNA
tattctctctcggtgcttggcct

SEQ ID NO: 91 - RNA, 21 nt, synthetic construct
  source 1..21: mol_type = other RNA
gatccggtcccaggttatgaa

SEQ ID NO: 92 - RNA, 23 nt, synthetic construct
  source 1..23: mol_type = other RNA
ttcataacctgggaccggatctg

SEQ ID NO: 93 - RNA, 21 nt, synthetic construct
  source 1..21: mol_type = other RNA
aagcaccgagagagaatgtca

SEQ ID NO: 94 - RNA, 23 nt, synthetic construct
  source 1..23: mol_type = other RNA
tgacattctctctcggtgcttgg

SEQ ID NO: 95 - RNA, 21 nt, synthetic construct
  source 1..21: mol_type = other RNA
ccatccagaactggtgcaaga

SEQ ID NO: 96 - RNA, 23 nt, synthetic construct
  source 1..23: mol_type = other RNA
tcttgcaccagttctggatggtc

SEQ ID NO: 97 - RNA, 21 nt, synthetic construct
  source 1..21: mol_type = other RNA
gtccaagatgcagcagaacga

SEQ ID NO: 98 - RNA, 23 nt, synthetic construct
  source 1..23: mol_type = other RNA
tcgttctgctgcatcttggacag

SEQ ID NO: 99 - RNA, 21 nt, synthetic construct
  source 1..21: mol_type = other RNA
caggtcatgagagaatgggaa

SEQ ID NO: 100 - RNA, 23 nt, synthetic construct
  source 1..23: mol_type = other RNA
ttcccattctctcatgacctggg

SEQ ID NO: 101 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agcaaaaccattgcttcacta

SEQ ID NO: 102 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 103 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagcaaaaccattgcttcaca

SEQ ID NO: 104 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 105 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = gm
  modified_base 5: mod_base = cm
  modified_base 6: mod_base = cm
  modified_base 7: mod_base = OTHER; note = 2'-fluorouridine
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gatgcctgaacttgaattaaa

SEQ ID NO: 106 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorouridine
  modified_base 7: mod_base = um
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tttaattcaagttcaggcatcta

SEQ ID NO: 107 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = um
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ctgtaacacaagtagatgcca

SEQ ID NO: 108 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = cm
  modified_base 8: mod_base = um
  modified_base 9: mod_base = OTHER; note = 2'-O-methyladenosine
  modified_base 10: mod_base = cm
  modified_base 11: mod_base = um
  modified_base 12: mod_base = um
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tggcatctacttgtgttacagca

SEQ ID NO: 109 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = um
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = um
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
cctgatcactatgcattttaa

SEQ ID NO: 110 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = gm
  modified_base 9: mod_base = cm
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaaaatgcatagtgatcaggaa

SEQ ID NO: 111 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = um
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = gm
  modified_base 15: mod_base = um
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tgacagctgtgctgtaacaca

SEQ ID NO: 112 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = um
  modified_base 6: mod_base = OTHER; note = 2'-fluorouridine
  modified_base 7: mod_base = OTHER; note = 2'-O-methyladenosine
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tgtgttacagcacagctgtcaaa

SEQ ID NO: 113 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = cm
  modified_base 16: mod_base = um
  modified_base 17: mod_base = cm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
catgaatagattctctcctga

SEQ ID NO: 114 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = gm
  modified_base 6: mod_base = OTHER; note = 2'-fluoroadenosine
  modified_base 7: mod_base = gm
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tcaggagagaatctattcatgca

SEQ ID NO: 115 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gtacagaatcattgcttatga

SEQ ID NO: 116 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = um
  modified_base 18: mod_base = gm
  modified_base 19: mod_base = um
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tcataagcaatgattctgtacaa

SEQ ID NO: 117 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-fluorocytidine
  modified_base 8: mod_base = gm
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorouridine
  modified_base 12: mod_base = cm
  modified_base 13: mod_base = um
  modified_base 14: mod_base = um
  modified_base 15: mod_base = um
  modified_base 16: mod_base = gm
  modified_base 17: mod_base = gm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tgtccacgtatctttgggtca

SEQ ID NO: 118 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-fluorocytidine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = um
  modified_base 13: mod_base = OTHER; note = 2'-O-methyladenosine
  modified_base 14: mod_base = OTHER; note = 2'-fluorocytidine
  modified_base 15: mod_base = gm
  modified_base 16: mod_base = OTHER; note = 2'-fluorouridine
  modified_base 17: mod_base = gm
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tgacccaaagatacgtggacaaa

SEQ ID NO: 119 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = cm
  modified_base 7: mod_base = OTHER; note = 2'-fluoroguanosine
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = um
  modified_base 14: mod_base = um
  modified_base 15: mod_base = gm
  modified_base 16: mod_base = gm
  modified_base 17: mod_base = gm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gtccacgtatctttgggtcta

SEQ ID NO: 120 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluorocytidine
  modified_base 6: mod_base = cm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluoroguanosine
  modified_base 17: mod_base = um
  modified_base 18: mod_base = gm
  modified_base 19: mod_base = gm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tagacccaaagatacgtggacaa

SEQ ID NO: 121 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = OTHER; note = 2'-fluoroguanosine
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = gm
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gatataggaattaagaggata

SEQ ID NO: 122 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorocytidine
  modified_base 6: mod_base = um
  modified_base 7: mod_base = OTHER; note = 2'-fluorocytidine
  modified_base 8: mod_base = um
  modified_base 9: mod_base = um
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorocytidine
  modified_base 15: mod_base = cm
  modified_base 16: mod_base = OTHER; note = 2'-fluorouridine
  modified_base 17: mod_base = OTHER; note = 2'-O-methyladenosine
  modified_base 18: mod_base = um
  modified_base 19: mod_base = OTHER; note = 2'-O-methyladenosine
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = cm
tatcctcttaattcctatatcac

SEQ ID NO: 123 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = gm
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = um
  modified_base 15: mod_base = cm
  modified_base 16: mod_base = um
  modified_base 17: mod_base = gm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
acagattgctgcttctgctaa

SEQ ID NO: 124 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine
  modified_base 3: mod_base = OTHER; note = 2'-O-methyladenosine
  modified_base 4: mod_base = gm
  modified_base 5: mod_base = OTHER; note = 2'-fluorocytidine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = gm
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = cm
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = cm
ttagcagaagcagcaatctgtac

SEQ ID NO: 125 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-O-methyladenosine
  modified_base 7: mod_base = OTHER; note = 2'-fluorouridine
  modified_base 8: mod_base = gm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroguanosine
  modified_base 12: mod_base = cm
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = cm
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ccaagatgcagcagaacggca

SEQ ID NO: 126 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = gm
  modified_base 6: mod_base = OTHER; note = 2'-fluorouridine
  modified_base 7: mod_base = um
  modified_base 8: mod_base = cm
  modified_base 9: mod_base = um
  modified_base 10: mod_base = gm
  modified_base 11: mod_base = cm
  modified_base 12: mod_base = um
  modified_base 13: mod_base = gm
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorouridine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = cm
tgccgttctgctgcatcttggac

SEQ ID NO: 127 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = cm
  modified_base 15: mod_base = gm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
aggaagcagccaacgagagaa

SEQ ID NO: 128 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = um
  modified_base 5: mod_base = cm
  modified_base 6: mod_base = OTHER; note = 2'-fluorouridine
  modified_base 7: mod_base = cm
  modified_base 8: mod_base = gm
  modified_base 9: mod_base = um
  modified_base 10: mod_base = um
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = cm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
ttctctcgttggctgcttcctgt

SEQ ID NO: 129 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = gm
  modified_base 14: mod_base = cm
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
ctttgagcagatgcagaacta

SEQ ID NO: 130 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = um
  modified_base 6: mod_base = OTHER; note = 2'-fluorocytidine
  modified_base 7: mod_base = um
  modified_base 8: mod_base = gm
  modified_base 9: mod_base = cm
  modified_base 10: mod_base = OTHER; note = 2'-O-methyladenosine
  modified_base 11: mod_base = um
  modified_base 12: mod_base = cm
  modified_base 13: mod_base = um
  modified_base 14: mod_base = OTHER; note = 2'-fluoroguanosine
  modified_base 15: mod_base = cm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tagttctgcatctgctcaaagaa

SEQ ID NO: 131 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = gm
  modified_base 6: mod_base = um
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ggcagttatccagcatttcca

SEQ ID NO: 132 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tggaaatgctggataactgcctt

SEQ ID NO: 133 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = OTHER; note = 2'-fluorocytidine
  modified_base 8: mod_base = um
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluoroadenosine
  modified_base 12: mod_base = OTHER; note = 2'-O-methyladenosine
  modified_base 13: mod_base = gm
  modified_base 14: mod_base = um
  modified_base 15: mod_base = um
  modified_base 16: mod_base = cm
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tccaacctacaagttctttga

SEQ ID NO: 134 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyladenosine
  modified_base 9: mod_base = cm
  modified_base 10: mod_base = um
  modified_base 11: mod_base = um
  modified_base 12: mod_base = gm
  modified_base 13: mod_base = um
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = um
  modified_base 18: mod_base = um
  modified_base 19: mod_base = gm
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tcaaagaacttgtaggttggatt

SEQ ID NO: 135 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = um
  modified_base 14: mod_base = um
  modified_base 15: mod_base = cm
  modified_base 16: mod_base = um
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
ccaacctacaagttctttgaa

SEQ ID NO: 136 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = OTHER; note = 2'-O-methyladenosine
  modified_base 9: mod_base = OTHER; note = 2'-O-methyladenosine
  modified_base 10: mod_base = cm
  modified_base 11: mod_base = um
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = um
ttcaaagaacttgtaggttggat

SEQ ID NO: 137 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = cm
  modified_base 14: mod_base = OTHER; note = 2'-O-methyladenosine
  modified_base 15: mod_base = gm
  modified_base 16: mod_base = um
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ctgaagaagaaacagtacaca

SEQ ID NO: 138 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = um
  modified_base 6: mod_base = OTHER; note = 2'-fluoroadenosine
  modified_base 7: mod_base = cm
  modified_base 8: mod_base = um
  modified_base 9: mod_base = gm
  modified_base 10: mod_base = um
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = um
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgtgtactgtttcttcttcagca

SEQ ID NO: 139 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorouridine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
gacaaagtagtagaagtagca

SEQ ID NO: 140 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-O-methyladenosine
  modified_base 6: mod_base = OTHER; note = 2'-fluorocytidine
  modified_base 7: mod_base = um
  modified_base 8: mod_base = um
  modified_base 9: mod_base = cm
  modified_base 10: mod_base = um
  modified_base 11: mod_base = OTHER; note = 2'-O-methyladenosine
  modified_base 12: mod_base = cm
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tgctacttctactactttgtctt

SEQ ID NO: 141 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = um
  modified_base 5: mod_base = um
  modified_base 6: mod_base = cm
  modified_base 7: mod_base = OTHER; note = 2'-fluorouridine
  modified_base 8: mod_base = um
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = gm
  modified_base 13: mod_base = cm
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
aagttctttgagcagatgcaa

SEQ ID NO: 142 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = cm
  modified_base 8: mod_base = um
  modified_base 9: mod_base = gm
  modified_base 10: mod_base = cm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
ttgcatctgctcaaagaacttgt

SEQ ID NO: 143 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorouridine
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = cm
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aggcagttatccagcatttca

SEQ ID NO: 144 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = cm
  modified_base 9: mod_base = um
  modified_base 10: mod_base = gm
  modified_base 11: mod_base = gm
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = gm
  modified_base 19: mod_base = cm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = cm
tgaaatgctggataactgccttc

SEQ ID NO: 145 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroguanosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = gm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
agcaccgagagagaatgtcca

SEQ ID NO: 146 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-fluoroadenosine
  modified_base 7: mod_base = um
  modified_base 8: mod_base = um
  modified_base 9: mod_base = cm
  modified_base 10: mod_base = um
  modified_base 11: mod_base = cm
  modified_base 12: mod_base = um
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroguanosine
  modified_base 17: mod_base = gm
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = gm
tggacattctctctcggtgcttg

SEQ ID NO: 147 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = gm
  modified_base 5: mod_base = um
  modified_base 6: mod_base = cm
  modified_base 7: mod_base = OTHER; note = 2'-fluorocytidine
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroguanosine
  modified_base 12: mod_base = um
  modified_base 13: mod_base = um
  modified_base 14: mod_base = OTHER; note = 2'-O-methyladenosine
  modified_base 15: mod_base = um
  modified_base 16: mod_base = gm
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ccggtcccaggttatgacaca

SEQ ID NO: 148 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = um
  modified_base 6: mod_base = OTHER; note = 2'-fluorocytidine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = cm
  modified_base 13: mod_base = um
  modified_base 14: mod_base = OTHER; note = 2'-fluoroguanosine
  modified_base 15: mod_base = gm
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = cm
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = um
tgtgtcataacctgggaccggat

SEQ ID NO: 149 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorouridine
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = um
  modified_base 14: mod_base = cm
  modified_base 15: mod_base = cm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aaccagtgaccatccagaaca

SEQ ID NO: 150 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = cm
  modified_base 6: mod_base = OTHER; note = 2'-fluorouridine
  modified_base 7: mod_base = gm
  modified_base 8: mod_base = gm
  modified_base 9: mod_base = OTHER; note = 2'-O-methyladenosine
  modified_base 10: mod_base = um
  modified_base 11: mod_base = gm
  modified_base 12: mod_base = gm
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = gm
  modified_base 19: mod_base = gm
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = gm
tgttctggatggtcactggttgg

SEQ ID NO: 151 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = gm
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = cm
  modified_base 14: mod_base = cm
  modified_base 15: mod_base = OTHER; note = 2'-O-methyladenosine
  modified_base 16: mod_base = cm
  modified_base 17: mod_base = um
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
accgaggactgaccactcgaa

SEQ ID NO: 152 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = OTHER; note = 2'-fluoroguanosine
  modified_base 7: mod_base = um
  modified_base 8: mod_base = gm
  modified_base 9: mod_base = gm
  modified_base 10: mod_base = um
  modified_base 11: mod_base = cm
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = cm
  modified_base 16: mod_base = OTHER; note = 2'-fluorocytidine
  modified_base 17: mod_base = um
  modified_base 18: mod_base = cm
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = gm
ttcgagtggtcagtcctcggtcg

SEQ ID NO: 153 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluoroguanosine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cacaccgtcgccaaagagaca

SEQ ID NO: 154 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluorocytidine
  modified_base 7: mod_base = um
  modified_base 8: mod_base = um
  modified_base 9: mod_base = um
  modified_base 10: mod_base = gm
  modified_base 11: mod_base = gm
  modified_base 12: mod_base = cm
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluoroguanosine
  modified_base 17: mod_base = gm
  modified_base 18: mod_base = um
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = cm
tgtctctttggcgacggtgtgcc

SEQ ID NO: 155 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroguanosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gccaagcaccgagagagaata

SEQ ID NO: 156 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = um
  modified_base 4: mod_base = um
  modified_base 5: mod_base = cm
  modified_base 6: mod_base = OTHER; note = 2'-fluorouridine
  modified_base 7: mod_base = cm
  modified_base 8: mod_base = um
  modified_base 9: mod_base = cm
  modified_base 10: mod_base = um
  modified_base 11: mod_base = cm
  modified_base 12: mod_base = gm
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = gm
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
tattctctctcggtgcttggcct

SEQ ID NO: 157 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = cm
  modified_base 5: mod_base = cm
  modified_base 6: mod_base = gm
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = gm
  modified_base 15: mod_base = um
  modified_base 16: mod_base = um
  modified_base 17: mod_base = OTHER; note = 2'-O-methyladenosine
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gatccggtcccaggttatgaa

SEQ ID NO: 158 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = cm
  modified_base 9: mod_base = cm
  modified_base 10: mod_base = um
  modified_base 11: mod_base = gm
  modified_base 12: mod_base = gm
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = gm
  modified_base 18: mod_base = gm
  modified_base 19: mod_base = OTHER; note = 2'-O-methyladenosine
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = gm
ttcataacctgggaccggatctg

SEQ ID NO: 159 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = cm
  modified_base 7: mod_base = OTHER; note = 2'-fluorocytidine
  modified_base 8: mod_base = gm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aagcaccgagagagaatgtca

SEQ ID NO: 160 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = um
  modified_base 8: mod_base = cm
  modified_base 9: mod_base = um
  modified_base 10: mod_base = cm
  modified_base 11: mod_base = um
  modified_base 12: mod_base = cm
  modified_base 13: mod_base = um
  modified_base 14: mod_base = OTHER; note = 2'-fluorocytidine
  modified_base 15: mod_base = gm
  modified_base 16: mod_base = OTHER; note = 2'-fluoroguanosine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = gm
tgacattctctctcggtgcttgg

SEQ ID NO: 161 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = cm
  modified_base 7: mod_base = OTHER; note = 2'-fluoroadenosine
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = gm
  modified_base 14: mod_base = gm
  modified_base 15: mod_base = um
  modified_base 16: mod_base = gm
  modified_base 17: mod_base = cm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ccatccagaactggtgcaaga

SEQ ID NO: 162 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = gm
  modified_base 6: mod_base = OTHER; note = 2'-fluorocytidine
  modified_base 7: mod_base = OTHER; note = 2'-O-methyladenosine
  modified_base 8: mod_base = cm
  modified_base 9: mod_base = cm
  modified_base 10: mod_base = OTHER; note = 2'-O-methyladenosine
  modified_base 11: mod_base = gm
  modified_base 12: mod_base = um
  modified_base 13: mod_base = um
  modified_base 14: mod_base = OTHER; note = 2'-fluorocytidine
  modified_base 15: mod_base = um
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tcttgcaccagttctggatggtc

SEQ ID NO: 163 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = cm
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gtccaagatgcagcagaacga

SEQ ID NO: 164 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = um
  modified_base 6: mod_base = OTHER; note = 2'-fluorocytidine
  modified_base 7: mod_base = um
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = gm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = gm
tcgttctgctgcatcttggacag

SEQ ID NO: 165 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = gm
  modified_base 5: mod_base = um
  modified_base 6: mod_base = cm
  modified_base 7: mod_base = OTHER; note = 2'-fluoroadenosine
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroguanosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = gm
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
caggtcatgagagaatgggaa

SEQ ID NO: 166 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = cm
  modified_base 5: mod_base = cm
  modified_base 6: mod_base = OTHER; note = 2'-fluoroadenosine
  modified_base 7: mod_base = um
  modified_base 8: mod_base = um
  modified_base 9: mod_base = cm
  modified_base 10: mod_base = um
  modified_base 11: mod_base = cm
  modified_base 12: mod_base = um
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = gm
ttcccattctctcatgacctggg

SEQ ID NO: 167 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 168 - DNA, 3583 bp, Homo sapiens
  source 1..3583: mol_type = genomic DNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagca

SEQ ID NO: 169 - protein, 681 aa, synthetic construct
HHHHHHCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 170 - protein, 678 aa, synthetic construct
HHHHHHCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 171 - protein, 193 aa, synthetic construct
HHHHHHHHGKPIPNPLLGLDSTGGGGSDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGGGGGGLPNIPVQTISRAAAEKLFGNMEGDCPSDWKTDSTCRMVTSESKNVKLTVS

SEQ ID NO: 172 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agcaaaaccattgcttcacta

SEQ ID NO: 173 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 174 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagcaaaaccattgcttcaca

SEQ ID NO: 175 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-O-fluoroguanosine with a 3'-phosphorothioate linkage
  modified_base 3: mod_base = um
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 176 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 177 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 178 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-fluorouridine
  modified_base 5: mod_base = gm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 179 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 180 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = OTHER; note = 2'-fluoroguanosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 181 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agcaaaaccattgcttcacta

SEQ ID NO: 182 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-fluorouridine
  modified_base 5: mod_base = gm
  modified_base 6: mod_base = OTHER; note = 2'-fluoroadenosine
  modified_base 7: mod_base = OTHER; note = 2'-fluoroadenosine
  modified_base 8: mod_base = gm
  modified_base 9: mod_base = OTHER; note = 2'-fluorocytidine
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 183 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = n is an abasic moiety
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agcaaaaccnttgcttcacta

SEQ ID NO: 184 - RNA, 21 nt, synthetic construct
  source 1..21: mol_type = other RNA
  modified_base 10: mod_base = OTHER; note = n is an abasic moiety
agcaaaaccnttgcttcacta

SEQ ID NO: 185 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-fluoroadenosine
  modified_base 7: mod_base = OTHER; note = 2'-fluoroadenosine
  modified_base 8: mod_base = OTHER; note = 2'-fluoroguanosine
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 186 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = 2'-deoxyadenosine
  modified_base 11: mod_base = OTHER; note = 2'-fluorouridine
  modified_base 12: mod_base = um
  modified_base 13: mod_base = gm
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
agcaaaaccattgcttcacta

SEQ ID NO: 187 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorocytidine
  modified_base 10: mod_base = OTHER; note = n is an abasic moiety
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagcaaaacnattgcttcaca

SEQ ID NO: 188 - RNA, 21 nt, synthetic construct
  source 1..21: mol_type = other RNA
  modified_base 10: mod_base = OTHER; note = n is an abasic moiety
cagcaaaacnattgcttcaca

SEQ ID NO: 189 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = gm
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 190 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-fluoroguanosine
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 191 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 192 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-fluorouridine
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 193 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagcaaaaccattgcttcaca

SEQ ID NO: 194 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-fluoroguanosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-fluoroguanosine
  modified_base 8: mod_base = cm
  modified_base 9: mod_base = OTHER; note = 2'-fluoroadenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 21: mod_base = OTHER; note = 2'-O-methyluridine with a 3'-phosphorothioate linkage
  modified_base 23: mod_base = cm
tgtgaagcaatggttttgctgtc

SEQ ID NO: 195 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-fluoroadenosine
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 196 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-fluoroadenosine
  modified_base 6: mod_base = OTHER; note = 2'-fluoroadenosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = cm
tgtgaagcaatggttttgctgtc

SEQ ID NO: 197 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-deoxycytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagcaaaaccattgcttcaca

SEQ ID NO: 198 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = gm
  modified_base 5: mod_base = cm
  modified_base 6: mod_base = cm
  modified_base 7: mod_base = um
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gatgcctgaacttgaattaaa

SEQ ID NO: 199 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-fluoroadenosine
  modified_base 6: mod_base = um
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
tttaattcaagttcaggcatcta

SEQ ID NO: 200 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = cm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = um
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ctgtaacacaagtagatgcca

SEQ ID NO: 201 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = cm
  modified_base 5: mod_base = OTHER; note = 2'-fluoroadenosine
  modified_base 6: mod_base = um
  modified_base 7: mod_base = OTHER; note = 2'-fluorocytidine
  modified_base 8: mod_base = um
  modified_base 9: mod_base = OTHER; note = 2'-O-methyladenosine
  modified_base 10: mod_base = cm
  modified_base 11: mod_base = um
  modified_base 12: mod_base = um
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tggcatctacttgtgttacagca

SEQ ID NO: 202 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = um
  modified_base 7: mod_base = cm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroadenosine
  modified_base 12: mod_base = um
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
cctgatcactatgcattttaa

SEQ ID NO: 203 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluorouridine
  modified_base 8: mod_base = gm
  modified_base 9: mod_base = cm
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaaaatgcatagtgatcaggaa

SEQ ID NO: 204 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = um
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = gm
  modified_base 15: mod_base = um
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tgacagctgtgctgtaacaca

SEQ ID NO: 205 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = gm
  modified_base 5: mod_base = OTHER; note = 2'-fluorouridine
  modified_base 6: mod_base = um
  modified_base 7: mod_base = OTHER; note = 2'-fluoroadenosine
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tgtgttacagcacagctgtcaaa

SEQ ID NO: 206 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = cm
  modified_base 16: mod_base = um
  modified_base 17: mod_base = cm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
catgaatagattctctcctga

SEQ ID NO: 207 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tcaggagagaatctattcatgca

SEQ ID NO: 208 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gtacagaatcattgcttatga

SEQ ID NO: 209 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = um
  modified_base 18: mod_base = gm
  modified_base 19: mod_base = um
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tcataagcaatgattctgtacaa

SEQ ID NO: 210 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = cm
  modified_base 6: mod_base = OTHER; note = 2'-O-methyladenosine
  modified_base 7: mod_base = cm
  modified_base 8: mod_base = gm
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluorouridine
  modified_base 12: mod_base = cm
  modified_base 13: mod_base = um
  modified_base 14: mod_base = um
  modified_base 15: mod_base = um
  modified_base 16: mod_base = gm
  modified_base 17: mod_base = gm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tgtccacgtatctttgggtca

SEQ ID NO: 211 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = cm
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = um
  modified_base 14: mod_base = um
  modified_base 15: mod_base = gm
  modified_base 16: mod_base = gm
  modified_base 17: mod_base = gm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gtccacgtatctttgggtcta

SEQ ID NO: 212 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = OTHER; note = 2'-O-methyladenosine
  modified_base 5: mod_base = um
  modified_base 6: mod_base = OTHER; note = 2'-O-methyladenosine
  modified_base 7: mod_base = gm
  modified_base 8: mod_base = gm
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorouridine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = gm
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gatataggaattaagaggata

SEQ ID NO: 213 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = um
  modified_base 7: mod_base = um
  modified_base 8: mod_base = gm
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = um
  modified_base 15: mod_base = cm
  modified_base 16: mod_base = um
  modified_base 17: mod_base = gm
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
acagattgctgcttctgctaa

SEQ ID NO: 214 - RNA, 22 nt, synthetic construct
  source 1..22: mol_type = other RNA
tagtgaagcaatggttttgctg

SEQ ID NO: 215 - RNA, 22 nt, synthetic construct
  source 1..22: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = gm
tagtgaagcaatggttttgctg

SEQ ID NO: 216 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = OTHER; note = 2'-O-methylcytidine with a 3'-phosphorothioate linkage
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
tagtgaagcaatggttttgctgt

SEQ ID NO: 217 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyluridine with a 3'-inverted abasic moiety connected via a 3' to 5' phosphodiester linkage
tagtgaagcaatggttttgctgt

SEQ ID NO: 218 - RNA, 22 nt, synthetic construct
  source 1..22: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = OTHER; note = 2'-O-methylcytidine with a 3'-phosphorothioate linkage
  modified_base 21: mod_base = OTHER; note = 2'-O-methyluridine with a 3'-phosphorothioate linkage
  modified_base 22: mod_base = gm
tagtgaagcaatggttttgctg

SEQ ID NO: 219 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-O-methyladenosine
  modified_base 7: mod_base = um
  modified_base 8: mod_base = gm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroguanosine
  modified_base 12: mod_base = cm
  modified_base 13: mod_base = OTHER; note = 2'-O-methyladenosine
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = cm
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ccaagatgcagcagaacggca

SEQ ID NO: 220 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluoroguanosine
  modified_base 6: mod_base = um
  modified_base 7: mod_base = OTHER; note = 2'-fluorouridine
  modified_base 8: mod_base = cm
  modified_base 9: mod_base = um
  modified_base 10: mod_base = gm
  modified_base 11: mod_base = cm
  modified_base 12: mod_base = um
  modified_base 13: mod_base = gm
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorouridine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = cm
tgccgttctgctgcatcttggac

SEQ ID NO: 221 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = cm
  modified_base 15: mod_base = gm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
aggaagcagccaacgagagaa

SEQ ID NO: 222 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = um
  modified_base 5: mod_base = OTHER; note = 2'-fluorocytidine
  modified_base 6: mod_base = um
  modified_base 7: mod_base = OTHER; note = 2'-fluorocytidine
  modified_base 8: mod_base = gm
  modified_base 9: mod_base = um
  modified_base 10: mod_base = um
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = cm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = um
ttctctcgttggctgcttcctgt

SEQ ID NO: 223 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = um
  modified_base 5: mod_base = gm
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = gm
  modified_base 14: mod_base = cm
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
ctttgagcagatgcagaacta

SEQ ID NO: 224 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = OTHER; note = 2'-fluorouridine
  modified_base 6: mod_base = cm
  modified_base 7: mod_base = OTHER; note = 2'-fluorouridine
  modified_base 8: mod_base = gm
  modified_base 9: mod_base = cm
  modified_base 10: mod_base = OTHER; note = 2'-O-methyladenosine
  modified_base 11: mod_base = um
  modified_base 12: mod_base = cm
  modified_base 13: mod_base = um
  modified_base 14: mod_base = OTHER; note = 2'-fluoroguanosine
  modified_base 15: mod_base = cm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tagttctgcatctgctcaaagaa

SEQ ID NO: 225 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = gm
  modified_base 6: mod_base = um
  modified_base 7: mod_base = um
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = cm
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ggcagttatccagcatttcca

SEQ ID NO: 226 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluorouridine
  modified_base 8: mod_base = gm
  modified_base 9: mod_base = cm
  modified_base 10: mod_base = um
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroadenosine
  modified_base 17: mod_base = cm
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tggaaatgctggataactgcctt

SEQ ID NO: 227 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorouridine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = gm
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
gacaaagtagtagaagtagca

SEQ ID NO: 228 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-fluoroadenosine
  modified_base 6: mod_base = cm
  modified_base 7: mod_base = OTHER; note = 2'-fluorouridine
  modified_base 8: mod_base = um
  modified_base 9: mod_base = cm
  modified_base 10: mod_base = um
  modified_base 11: mod_base = OTHER; note = 2'-O-methyladenosine
  modified_base 12: mod_base = cm
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tgctacttctactactttgtctt

SEQ ID NO: 229 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = gm
  modified_base 7: mod_base = um
  modified_base 8: mod_base = um
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = cm
  modified_base 16: mod_base = OTHER; note = 2'-O-methyladenosine
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aggcagttatccagcatttca

SEQ ID NO: 230 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroadenosine
  modified_base 6: mod_base = um
  modified_base 7: mod_base = OTHER; note = 2'-fluoroguanosine
  modified_base 8: mod_base = cm
  modified_base 9: mod_base = um
  modified_base 10: mod_base = gm
  modified_base 11: mod_base = gm
  modified_base 12: mod_base = OTHER; note = 2'-O-methyladenosine
  modified_base 13: mod_base = um
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = gm
  modified_base 19: mod_base = cm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = cm
tgaaatgctggataactgccttc

SEQ ID NO: 231 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = gm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroguanosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = gm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
agcaccgagagagaatgtcca

SEQ ID NO: 232 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluorocytidine
  modified_base 6: mod_base = OTHER; note = 2'-O-methyladenosine
  modified_base 7: mod_base = OTHER; note = 2'-fluorouridine
  modified_base 8: mod_base = um
  modified_base 9: mod_base = cm
  modified_base 10: mod_base = um
  modified_base 11: mod_base = cm
  modified_base 12: mod_base = um
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroguanosine
  modified_base 17: mod_base = gm
  modified_base 18: mod_base = um
  modified_base 19: mod_base = gm
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = gm
tggacattctctctcggtgcttg

SEQ ID NO: 233 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = gm
  modified_base 7: mod_base = um
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = um
  modified_base 14: mod_base = cm
  modified_base 15: mod_base = cm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
aaccagtgaccatccagaaca

SEQ ID NO: 234 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = OTHER; note = 2'-fluorocytidine
  modified_base 6: mod_base = um
  modified_base 7: mod_base = OTHER; note = 2'-fluoroguanosine
  modified_base 8: mod_base = gm
  modified_base 9: mod_base = OTHER; note = 2'-O-methyladenosine
  modified_base 10: mod_base = um
  modified_base 11: mod_base = gm
  modified_base 12: mod_base = gm
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorocytidine
  modified_base 17: mod_base = um
  modified_base 18: mod_base = gm
  modified_base 19: mod_base = gm
  modified_base 20: mod_base = um
  modified_base 21: mod_base = OTHER; note = 2'-O-methyluridine with a 3'-phosphorothioate linkage
  modified_base 22: mod_base = OTHER; note = 2'-O-methylguanosine with a 3'-phosphorothioate linkage
  modified_base 23: mod_base = gm
tgttctggatggtcactggttgg

SEQ ID NO: 235 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = gm
  modified_base 7: mod_base = gm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluoroguanosine
  modified_base 12: mod_base = OTHER; note = 2'-O-methyladenosine
  modified_base 13: mod_base = cm
  modified_base 14: mod_base = cm
  modified_base 15: mod_base = OTHER; note = 2'-O-methyladenosine
  modified_base 16: mod_base = cm
  modified_base 17: mod_base = um
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
accgaggactgaccactcgaa

SEQ ID NO: 236 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = gm
  modified_base 5: mod_base = OTHER; note = 2'-fluoroadenosine
  modified_base 6: mod_base = gm
  modified_base 7: mod_base = OTHER; note = 2'-fluorouridine
  modified_base 8: mod_base = gm
  modified_base 9: mod_base = gm
  modified_base 10: mod_base = um
  modified_base 11: mod_base = cm
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = cm
  modified_base 16: mod_base = OTHER; note = 2'-fluorocytidine
  modified_base 17: mod_base = um
  modified_base 18: mod_base = cm
  modified_base 19: mod_base = gm
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = gm
ttcgagtggtcagtcctcggtcg

SEQ ID NO: 237 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = cm
  modified_base 6: mod_base = cm
  modified_base 7: mod_base = gm
  modified_base 8: mod_base = um
  modified_base 9: mod_base = OTHER; note = 2'-fluorocytidine
  modified_base 10: mod_base = OTHER; note = 2'-fluoroguanosine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cacaccgtcgccaaagagaca

SEQ ID NO: 238 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = cm
  modified_base 7: mod_base = OTHER; note = 2'-fluorouridine
  modified_base 8: mod_base = um
  modified_base 9: mod_base = um
  modified_base 10: mod_base = gm
  modified_base 11: mod_base = gm
  modified_base 12: mod_base = cm
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluoroguanosine
  modified_base 17: mod_base = gm
  modified_base 18: mod_base = um
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = cm
tgtctctttggcgacggtgtgcc

SEQ ID NO: 239 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroguanosine
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
gccaagcaccgagagagaata

SEQ ID NO: 240 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluoroadenosine with a 3'-phosphorothioate linkage
  modified_base 3: mod_base = um
  modified_base 4: mod_base = um
  modified_base 5: mod_base = OTHER; note = 2'-fluorocytidine
  modified_base 6: mod_base = um
  modified_base 7: mod_base = OTHER; note = 2'-fluorocytidine
  modified_base 8: mod_base = um
  modified_base 9: mod_base = cm
  modified_base 10: mod_base = um
  modified_base 11: mod_base = cm
  modified_base 12: mod_base = gm
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = um
  modified_base 19: mod_base = gm
  modified_base 20: mod_base = gm
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
tattctctctcggtgcttggcct

SEQ ID NO: 241 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = cm
  modified_base 5: mod_base = cm
  modified_base 6: mod_base = gm
  modified_base 7: mod_base = gm
  modified_base 8: mod_base = um
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = gm
  modified_base 15: mod_base = um
  modified_base 16: mod_base = um
  modified_base 17: mod_base = OTHER; note = 2'-O-methyladenosine
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gatccggtcccaggttatgaa

SEQ ID NO: 242 - RNA, 23 nt, synthetic construct
  source 1..23: mol_type = other RNA
  modified_base 1: mod_base = OTHER; note = 2'-O-methyluridine with a 3'-phosphorothioate linkage and either a 5'-phosphate or a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = cm
  modified_base 10: mod_base = um
  modified_base 11: mod_base = gm
  modified_base 12: mod_base = gm
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = gm
  modified_base 18: mod_base = gm
  modified_base 19: mod_base = OTHER; note = 2'-O-methyladenosine
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = gm
ttcataacctgggaccggatctg

(19) United States
(12) Patent Application Publication
(10) Pub. No.: US 2025/0064958 A1
(43) Pub. Date: Feb. 27, 2025

(54) AMYLOID PRECURSOR PROTEIN (APP) RNAi AGENTS

(71) Applicant: ELI LILLY AND COMPANY, Indianapolis, IN (US)

(72) Inventors: Riazul ALAM, Fishers, IN (US); Jee-Wei Emily CHEN, Indianapolis, IN (US); Carrie Hughes CROY, Fishers, IN (US); David Albert DRIVER, Solana Beach, CA (US); Sarah Katharina FRITSCHI, Boston, MA (US); Daniel Scott GIRARD, San Marcos, CA (US); Isabel C. GONZALEZ VALCARCEL, Indianapolis, IN (US); Ruben Martin MARTINEZ, Indianapolis, IN (US); Rebecca Ruth MILES, Pendleton, IN (US); Hatice Gulcin OZER, Columbus, OH (US); Douglas Raymond PERKINS, New Palestine, IN (US); Jibo WANG, Carmel, IN (US)

(21) Appl. No.: 18/806,842

(22) Filed: Aug. 16, 2024

Related U.S. Application Data
(60) Provisional application No. 63/578,018, filed on Aug. 22, 2023.
(60) Provisional application No. 63/645,219, filed on May 10, 2024.

(51) Int. Cl. A61K 47/68; A61K 9/00; A61P 25/28; C12N 15/113

(52) U.S. Cl. CPC A61K 47/6807; A61K 9/0019; A61K 47/6889; A61P 25/28; C12N 15/113; C12N 2310/11; C12N 2310/14; C12N 2310/315; C12N 2310/321; C12N 2310/322

(57) ABSTRACT

Provided herein are APP RNAi agents and compositions comprising an APP RNAi agent. Also provided herein are methods of using the APP RNAi agents or compositions comprising an APP RNAi agent in reducing APP expression and/or treating APP associated neurological diseases.

### SEQUENCE LISTING

The present application is being filed along with a Sequence Listing in ST.26 XML format. The Sequence Listing is provided as a file titled “30578 WO” created Jun. 14, 2024, and is 1.576 megabytes in size. The Sequence Listing information in the ST.26 XML format is incorporated herein by reference in its entirety.

### BACKGROUND

Amyloid precursor protein (APP) is a transmembrane protein expressed in neurons and glia. APP is cleaved by β-secretase and γ-secretase to release the amyloid beta (Aβ) peptides, which encompass a group of peptides ranging in size of 38-43 amino acid residues. Aβ monomers aggregate into various types of higher order structures including oligomers, protofibrils and amyloid fibrils. Amyloid oligomers are soluble and may spread throughout the brain, while amyloid fibrils are larger and insoluble and can further aggregate to form amyloid deposits or plaques. Amyloid plaques in the brain have been associated with a number of conditions and diseases, including Alzheimer's disease (AD), Down's syndrome, and cerebral amyloid angiopathy (CAA).

The blood brain barrier (BBB) is a selective semipermeable border of capillary endothelial cells that prevents solutes, including pathogens, from passing into the central nervous system (CNS). The BBB allows the passage of some small molecules by passive diffusion and the cells of BBB actively transport metabolic products crucial to neural function such as glucose and amino acids across the barrier using specific transport proteins. The BBB has neuroprotective function by tightly controlling access to the brain; but it also impedes access of therapeutic agents to CNS. Antibodies directed to transferrin receptor (“TfR”) have been used for modulating BBB transport. However, attempts at using anti-TfR antibodies to shuttle therapeutic agents across the BBB have proven challenging. To date, there are no approved TfR shuttles or conjugates for the treatment of CNS diseases.

RNA interference (RNAi) is a highly conserved regulatory mechanism in which RNA molecules are involved in sequence-specific suppression of gene expression by double-stranded RNA molecules (dsRNA) (Fire et al., Nature 391:806-811, 1998).

Currently, there are no disease modifying treatments available for Down's syndrome and cerebral amyloid angiopathy. Although FDA recently approved two anti-Aβ antibodies (aducanumab and lecanemab) for treating AD, the AD patients vary widely in the progression of disease, initiation of symptoms, trajectory of cognitive and functional decline, and their response to treatment. Accordingly, there remains a need for therapeutic agents that can cross BBB and access the CNS and attack the initiation of amyloid cascade by inhibiting APP mRNA expression, e.g., by utilizing RNAi, and thereby reduce the production and/or level of disease causing Aβ peptides.

### SUMMARY OF INVENTION

Provided herein are APP RNAi agents and compositions comprising an APP RNAi agent that can access CNS and reduce APP mRNA expression. Also provided herein are methods of using the APP RNAi agents or compositions comprising an APP RNAi agent for reducing APP expression and/or treating APP associated neurological diseases.

In one aspect, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain (“human TfR binding protein”); wherein L is a linker, or optionally absent, and wherein n is an integer of 1 to 3. In some embodiments, n is 1. In some embodiments, n is 2. In some embodiments, n is 3.

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain; wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, VH comprises SEQ ID NO: 7 and VL comprises SEQ ID NO: 8. In some embodiments, VH comprises a sequence having at least 95% sequence identity to SEQ ID NO: 7 and VL comprises a sequence having at least 95% sequence identity to SEQ ID NO: 8. Exemplary sequences of human TfR binding domains and proteins are provided in Table 1a and 1b.

In some embodiments, L is a Mal-Tet-TCO linker, SMCC linker, or GDM linker (see Table 4). In some embodiments, L is a SMCC linker in Table 4.

In another aspect, provided herein are APP RNAi agents comprising a double stranded RNA (dsRNA) comprising a sense stand and an antisense strand, wherein the sense stand and antisense strand sequences are selected from Table 5a, 5b, 7a, 7b. In some embodiments, APP RNAi agents comprising any dsRNA in Table 5a, 5b, 7a, 7b.

Exemplary unmodified sense strand and antisense strand sequences of dsRNA targeting human APP mRNA are provided in Table 5a and 5b. In some embodiments, the sense strand and the antisense strand of the dsRNA comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40;
(d) the sense strand comprises SEQ ID NO: 41, and the antisense strand comprises SEQ ID NO: 42;
(e) the sense strand comprises SEQ ID NO: 43, and the antisense strand comprises SEQ ID NO: 44;
(f) the sense strand comprises SEQ ID NO: 45, and the antisense strand comprises SEQ ID NO: 46;
(g) the sense strand comprises SEQ ID NO: 47, and the antisense strand comprises SEQ ID NO: 48;
(h) the sense strand comprises SEQ ID NO: 49, and the antisense strand comprises SEQ ID NO: 50;
(i) the sense strand comprises SEQ ID NO: 51, and the antisense strand comprises SEQ ID NO: 52;
(j) the sense strand comprises SEQ ID NO: 53, and the antisense strand comprises SEQ ID NO: 54;
(k) the sense strand comprises SEQ ID NO: 55, and the antisense strand comprises SEQ ID NO: 56;
(l) the sense strand comprises SEQ ID NO: 57, and the antisense strand comprises SEQ ID NO: 58;
(m) the sense strand comprises SEQ ID NO: 59, and the antisense strand comprises SEQ ID NO: 60;
(n) the sense strand comprises SEQ ID NO: 61, and the antisense strand comprises SEQ ID NO: 62;
(o) the sense strand comprises SEQ ID NO: 63, and the antisense strand comprises SEQ ID NO: 64;
(p) the sense strand comprises SEQ ID NO: 65, and the antisense strand comprises SEQ ID NO: 66;
(q) the sense strand comprises SEQ ID NO: 67, and the antisense strand comprises SEQ ID NO: 68;
(r) the sense strand comprises SEQ ID NO: 69, and the antisense strand comprises SEQ ID NO: 70;
(s) the sense strand comprises SEQ ID NO: 71, and the antisense strand comprises SEQ ID NO: 72;
(t) the sense strand comprises SEQ ID NO: 73, and the antisense strand comprises SEQ ID NO: 74;
(u) the sense strand comprises SEQ ID NO: 75, and the antisense strand comprises SEQ ID NO: 76;
(v) the sense strand comprises SEQ ID NO: 77, and the antisense strand comprises SEQ ID NO: 78;
(w) the sense strand comprises SEQ ID NO: 79, and the antisense strand comprises SEQ ID NO: 80;
(x) the sense strand comprises SEQ ID NO: 81, and the antisense strand comprises SEQ ID NO: 82;
(y) the sense strand comprises SEQ ID NO: 83, and the antisense strand comprises SEQ ID NO: 84;
(z) the sense strand comprises SEQ ID NO: 85, and the antisense strand comprises SEQ ID NO: 86;
(aa) the sense strand comprises SEQ ID NO: 87, and the antisense strand comprises SEQ ID NO: 88;
(bb) the sense strand comprises SEQ ID NO: 89, and the antisense strand comprises SEQ ID NO: 90;
(cc) the sense strand comprises SEQ ID NO: 91, and the antisense strand comprises SEQ ID NO: 92;
(dd) the sense strand comprises SEQ ID NO: 93, and the antisense strand comprises SEQ ID NO: 94;
(ee) the sense strand comprises SEQ ID NO: 95, and the antisense strand comprises SEQ ID NO: 96;
(ff) the sense strand comprises SEQ ID NO: 97, and the antisense strand comprises SEQ ID NO: 98;
(gg) the sense strand comprises SEQ ID NO: 99, and the antisense strand comprises SEQ ID NO: 100;
(hh) the sense strand comprises SEQ ID NO: 184, and the antisense strand comprises SEQ ID NO: 36;
(ii) the sense strand comprises SEQ ID NO: 188, and the antisense strand comprises SEQ ID NO: 38; and
(jj) the sense strand comprises SEQ ID NO: 35, and the antisense strand comprises SEQ ID NO: 214;
   wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 35, and the antisense strand comprises SEQ ID NO: 36. In some embodiments, the sense strand comprises SEQ ID NO: 35, and the antisense strand comprises SEQ ID NO: 214. In some embodiments, the sense strand comprises SEQ ID NO: 37, and the antisense strand comprises SEQ ID NO: 38.

The dsRNA can include modifications. The modifications can be made to one or more nucleotides of the sense and/or antisense strand or to the internucleotide linkages. In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl modified nucleotide.

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human APP mRNA are provided in Table 7a and 7b.

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain, and P is selected from TBP1, TBP2, TBP3, TBP4, or TBP5 in Table 1b; wherein L is a linker, or optionally absent, and wherein n is 1 or 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the dsRNA is any dsRNA in Table 5a, 5b, 7a or 7b (e.g., dsRNA No. 1); wherein P is a protein comprising one monovalent human TfR binding domain; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the dsRNA is any dsRNA in Table 5a, 5b, 7a or 7b (e.g., dsRNA No. 1); wherein P is a protein comprising one monovalent human TfR binding domain, and P is selected from TBP1, TBP2, TBP3, TBP4, or TBP5 in Table 1b; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 14, LC1 comprises SEQ ID NO: 10, HC2 comprises SEQ ID NO: 15, and wherein n is 1 or 2. In some embodiments, n is 1. In some embodiments, n is 2. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 16, LC1 comprises SEQ ID NO: 10, HC2 comprises SEQ ID NO: 17, and wherein n is 1. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 35, and the antisense strand comprises SEQ ID NO: 36 or 214; wherein P is a protein comprising one monovalent human TfR binding domain; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 35, and the antisense strand comprises SEQ ID NO: 36 or 214; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 14, LC1 comprises SEQ ID NO: 10, HC2 comprises SEQ ID NO: 15, and wherein n is 1 or 2. In some embodiments, n is 1. In some embodiments, n is 2. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 35, and the antisense strand comprises SEQ ID NO: 36 or 214; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 16, LC1 comprises SEQ ID NO: 10, HC2 comprises SEQ ID NO: 17, and wherein n is 1. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 172, and the antisense strand comprises SEQ ID NO: 173 or 217; wherein P is a protein comprising one monovalent human TfR binding domain; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 172, and the antisense strand comprises SEQ ID NO: 173 or 217; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 14, LC1 comprises SEQ ID NO: 10, HC2 comprises SEQ ID NO: 15, and wherein n is 1 or 2. In some embodiments, n is 1. In some embodiments, n is 2. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, wherein the sense strand comprises SEQ ID NO: 172, and the antisense strand comprises SEQ ID NO: 173 or 217; wherein P is a protein comprising one monovalent human TfR binding domain; wherein L is a linker, or optionally absent, wherein the human TfR binding domain comprises two heavy chains HC1 and HC2 and one light chain LC1, wherein HC1 comprises SEQ ID NO: 16, LC1 comprises SEQ ID NO: 10, HC2 comprises SEQ ID NO: 17, and wherein n is 1. In some embodiments, L is a linker in Table 4 (e.g., a SMCC linker in Table 4).

In another aspect, provided herein are methods of treating an APP associated neurologic disease in a patient in need thereof, and such the method comprises administering to the patient an effective amount of the APP RNAi agent or a pharmaceutical composition described herein. In some embodiments, the APP associated neurological disease is selected from Alzheimer's disease, Down's syndrome, or cerebral amyloid angiopathy. The APP RNAi agent or a pharmaceutical composition comprising APP RNAi agent can be administered to the patient intravenously or subcutaneously.

In another aspect, provided herein are APP RNAi agents or pharmaceutical compositions comprising an APP RNAi agent for use in a therapy. Also provided herein are APP RNAi agents or pharmaceutical compositions comprising an APP RNAi agent for use in the treatment of an APP associated neurological disease, e.g., Alzheimer's disease, Down's syndrome, or cerebral amyloid angiopathy. Also provided herein are uses of the APP RNAi agent in the manufacture of a medicament for treating an APP associated neurological disease, e.g., Alzheimer's disease, Down's syndrome, or cerebral amyloid angiopathy.

### BRIEF DESCRIPTION OF THE DRAWINGS

FIG. 1A shows an exemplary analytical anion exchange (aAEX) chromatogram of DAR profile for TBP5-dsRNA No. 48 conjugate before purification. FIG. 1B shows an exemplary aAEX chromatogram of DAR profile for TBP5-dsRNA No. 48 conjugate after purification. FIG. 1C shows an exemplary analytical anion exchange (aAEX) chromatogram of DAR profile for TBP4-dsRNA No. 48 conjugate before purification. FIG. 1D shows an exemplary aAEX chromatogram of DAR profile for TBP4-dsRNA No. 48 conjugate after purification. FIG. 1E shows an exemplary analytical anion exchange (aAEX) chromatogram of DAR profile for TBP5-dsRNA No. 109 conjugate before purification. FIG. 1F shows an exemplary aAEX chromatogram of DAR profile for TBP5-dsRNA No. 109 conjugate after purification.

FIGS. 2A and 2B show in vitro potency of two APP RNAi agents (TBP4-dsRNA NO. 48 in 2A and TBP4-dsRNA NO. 50 in 2B) for knocking down human APP gene (mRNA) in EFO-21 cells. FIGS. 2C and 2D show in vitro potency of two APP RNAi agents (mTBP1-dsRNA NO. 48 in 2C and mTBP1-dsRNA NO. 50 in 2D) for knocking down mouse APP gene (mRNA) in mouse cortical neurons. FIG. 2C dose response curves generated the following IC50 fits: cholesterol conjugated APP dsRNA48 (IC50=0.749 nm); IsoAb-dsRNA48-DAR2 (IC50=28.3 nM); mTBP1-dsRNA48-DAR2 (IC50=0.558 nM). FIG. 2D dose response curves generated the following IC50 fits: cholesterol conjugated APP dsRNA50 (IC50=0.270 nM); IsoAb-dsRNA50-DAR2 (IC50=36.6 nM); mTBP1-dsRNA50-DAR2 (IC50=0.430 nM).

FIGS. 3A and 3B show in vivo efficacy of two APP RNAi agents after a single ICV dose of 30 ug in wild-type mice after 7 days. The two agents have distinct 2′ fluoro modification patterns, see Table 7a for dsRNA No. 48 and dsRNA No. 63. FIG. 3A shows APP gene (mRNA) knockdown and FIG. 3B shows Aβ, measures both Aβ(1-40) and Aβ(1-42), protein knockdown of the two specified RNAi agents in disease relevant cortical and hippocampal regions.

FIGS. 4A and 4B show the in vivo efficacy in the brain for two TfR binding protein APP siRNA conjugates: TBP4-dsRNA No. 48 (DAR2) or TBP5-sdRNA No. 48 (DAR1) 28 days after single (IV) dose of 10 mg/kg (effective dsRNA concentration) in hTfR transgenic-mouse. FIG. 4A shows APP mRNA reduction of APP RNAi agent TBP4-dsRNA No. 48 (DAR2) or TBP5-sdRNA No. 48 (DAR1). FIG. 4B shows the exposure of the antisense strand of the above APP RNAi agents in the mouse brain.

FIGS. 5A-5D show the in vivo efficacy in the brain for two TfR binding protein APP siRNA conjugates: TBP4-dsRNA No. 48 (DAR2) or TBP5-sdRNA No. 48 (DAR1), 28 days after single (IV) dose of 10 mg/kg (effective dsRNA concentration) in Cynomolgus monkey. FIG. 5A shows APP mRNA reduction in Cynomolgus monkey brain after a single intravenous (IV) dose of the APP RNAi agent. FIG. 5B shows Aβ, measures both Aβ(1-40) and Aβ(1-42), protein reduction after a single intravenous (IV) dose of the APP RNAi agent.

FIG. 5C shows the exposure of the antisense strand of APP RNAi agent in both central-nervous system (prefrontal cortex, hippocampus) and peripheral (spleen, kidney, liver, heart) tissues of Cynomolgus monkey at 28 days post dose. FIG. 5D shows the pharmacokinetic exposure profile of the specified reagent in the plasma, antibody-conjugated antisense strand concentration (nM).

FIGS. 6A-6D demonstrate the durability of the TfR binding protein APP siRNA conjugates, TBP5-sdRNA No. 48 (DAR1), efficacy 29, 92 or 181 days after a single (IV) dose of 10 mg/kg (effective dsRNA concentration) in Cynomolgus monkey. FIGS. 6A and 6C shows APP mRNA reduction (mean±SEM, n=3) in Cynomolgus monkey brain after a single intravenous (IV) dose of the APP RNAi agent in the prefrontal cortex (6A) and hippocampus (6C) respectively. FIGS. 6B and 6D shows Aβ, measures both Aβ(1-40) and Aβ(1-42), protein reduction (mean±SEM, n=3) after a single intravenous (IV) dose of the APP RNAi agent in the prefrontal cortex (6B) and hippocampus (6D) respectively.

FIGS. 7A and 7B show the in vivo efficacy of an APP RNAi agent after a single intravenous (IV) or subcutaneous (SC) dose of APP RNAi agent TBP5-sdRNA No. 48 (DAR1) in transgenic hTfR mice. The agent was dosed at either 3, 1, or 0.3 mg/kg (effective dsRNA concentration). FIG. 7A shows the APP mRNA reduction in the prefrontal cortex 28 days post dose; and FIG. 7B shows the APP mRNA reduction in the hippocampus 28 days post dose.

FIG. 8 shows the in vivo efficacy of an APP RNAi agent after a single intravenous (IV) dose of APP RNAi agent TBP5-dsRNA No. 48 (DAR1) or TBP5-dsRNA No. 109 (DAR1) in transgenic hTfR mice. TBP5-dsRNA No. 109 (DAR1) has an inverted abasic cap at the 3′ end of the antisense strand. The agents were dosed at 1 mg/kg (effective dsRNA concentration) IV into transgenic hTfR mice. FIG. 8 illustrates the level of APP mRNA reduction in hippocampus, prefrontal cortex and brain stem 29 or 84 days post dose.

FIGS. 9A and 9B demonstrate the potency and durability of the TfR binding protein APP siRNA conjugate, TBP5-dsRNA No. 109 (DAR1), which has an inverted abasic cap at the 3′ end of the antisense strand, after a single (IV) dose of 10 mg/kg (effective dsRNA concentration) in Cynomolgus monkey. FIG. 9A shows the APP mRNA reduction (mean, n=3-4) in disease relevant cortical (prefrontal, motor, parietal and temporal) and hippocampal regions 29 and 85 days post dose. FIG. 9B shows Aβ, measures both Aβ(1-40) and Aβ(1-42), protein reduction (mean, n=3-4) in disease relevant cortical (prefrontal, motor, parietal and temporal) and hippocampal regions 29 and 85 days post dose.

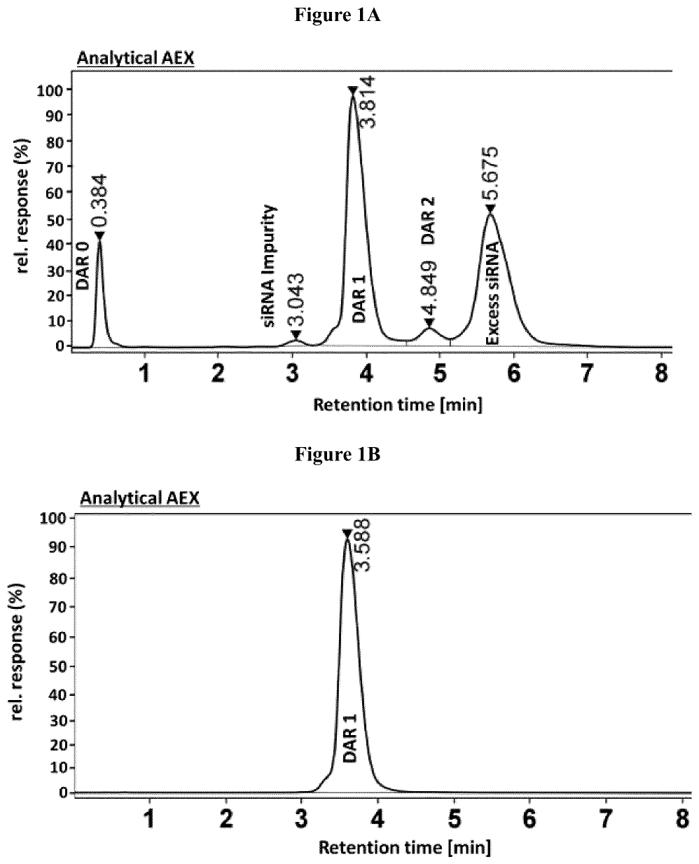

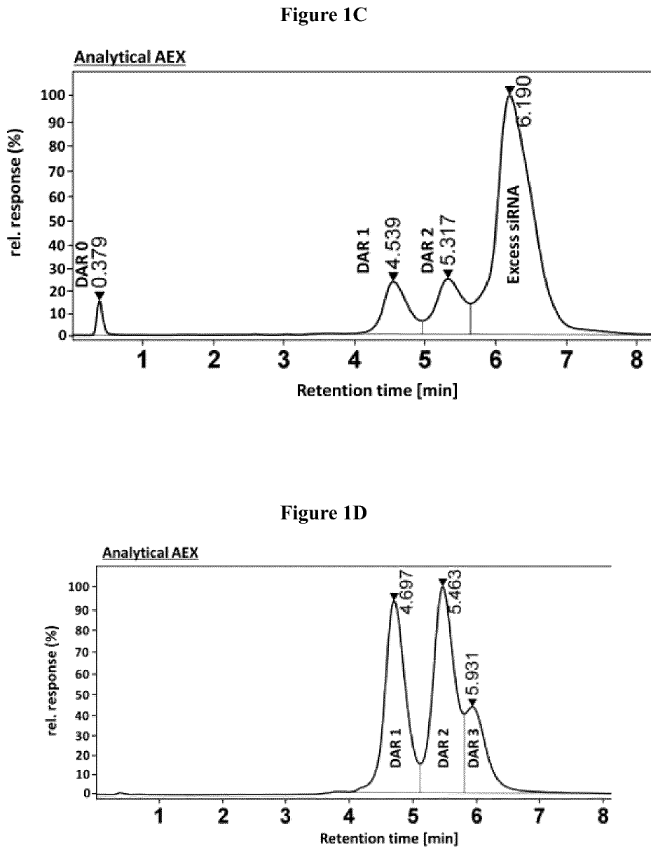

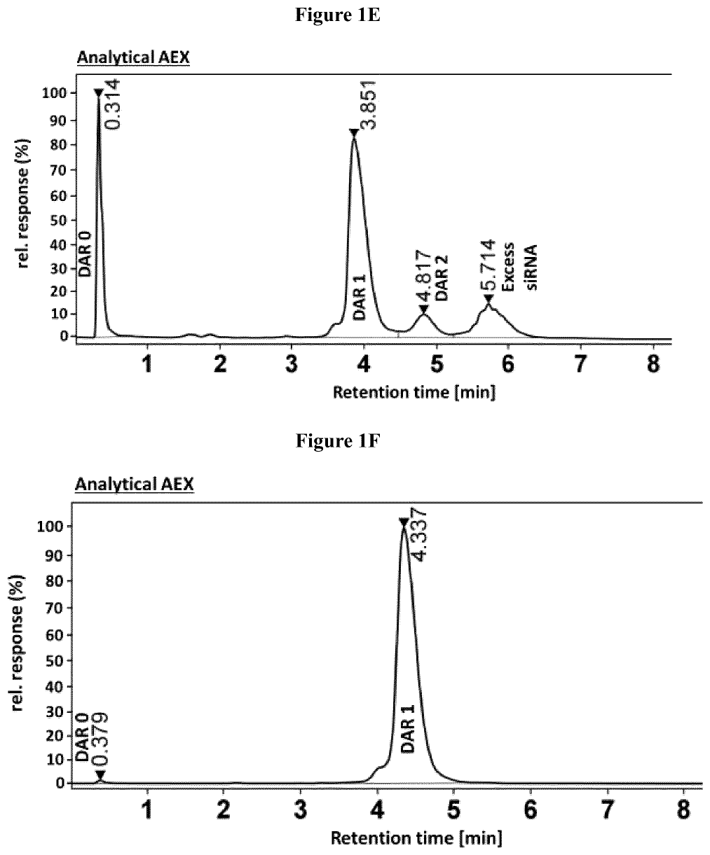

### DETAILED DESCRIPTION

Provided herein are APP RNAi agents and compositions comprising an APP RNAi agent that can access CNS and reduce APP mRNA expression. Also provided herein are methods of using the APP RNAi agents or compositions comprising an APP RNAi agent for reducing APP expression and/or treating APP associated neurological diseases.

In one aspect, provided herein are APP RNAi agents comprising Formula (I): (R-L)ₙ-P, wherein R is a double stranded RNA (dsRNA) comprising a sense stand and an antisense strand, wherein the antisense strand is complementary to APP mRNA; wherein P is a protein comprising one monovalent human TfR binding domain (“human TfR binding protein”); wherein L is a linker, or optionally absent, and wherein n is an integer of 1 to 3. In some embodiments, n is 1. In some embodiments, n is 2. In some embodiments, n is 3.

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain; wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the antisense strand is complementary to APP mRNA; wherein P is a protein comprising one monovalent human TfR binding domain, and P is selected from TBP1, TBP2, TBP3, TBP4, or TBP5 in Table 1b; wherein L is a linker, or optionally absent, and wherein n is 1 or 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the dsRNA is any dsRNA in Table 5a, 5b, 7a or 7b (e.g., dsRNA No. 1); wherein P is a protein comprising one monovalent human TfR binding domain; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In some embodiments, provided herein are APP RNAi agents comprising Formula (I): (R-L)ₙ-P, wherein R is a double stranded RNA (dsRNA) comprising a sense stand and an antisense strand, and the dsRNA is any dsRNA in Table 5a, 5b, 7a or 7b (e.g., dsRNA No. 1); wherein P is a protein comprising one monovalent human TfR binding domain, and P is selected from TBP1, TBP2, TBP3, TBP4, or TBP5 in Table 1b; wherein L is a linker, or optionally absent, and wherein n is an integer of 1 to 3. In some embodiments, n is 1. In some embodiments, n is 2. In some embodiments, n is 3. In some embodiments, L is a linker in Table 4 (e.g., a SMCC linker in Table 4).

In another aspect, provided herein are APP RNAi agents comprising a double stranded RNA (dsRNA) comprising a sense stand and an antisense strand, wherein the sense stand and antisense strand sequences are selected from Table 5a, 5b, 7a, 7b. In some embodiments, APP RNAi agents comprising any dsRNA in Table 5a, 5b, 7a, 7b.

### Human TfR Binding Proteins

The APP RNAi agents described herein comprise a protein comprising one monovalent human TfR binding domain (“human TfR binding protein”). Human TfR binding protein of the APP RNAi agents can bind TfR on BBB and transport the dsRNA into the CNS.

Exemplary sequences of human TfR binding domains and proteins are provided in Table 1a and 1b. In some embodiments, the monovalent human TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, HCDR1 comprises SEQ TD NO: 1, HCDR2 comprises SEQ TD NO: 2, HCDR3 comprises SEQ TD NO: 3, LCDR1 comprises SEQ ID NO: 4, LCDR2 comprises SEQ TD NO: 5, and LCDR3 comprises SEQ ID NO: 6. In some embodiments, VH comprises SEQ TD NO: 7, and VL comprises SEQ TD NO: 8. In some embodiments, VH comprises a sequence having at least 9500 sequence identity to SEQ ID NO: 7, and VL comprises a sequence having at least 95% sequence identity to SEQ TD NO: 8.

TABLE 1a
Exemplary sequences of human TfR binding domains and proteins

| Region | Sequence | SEQ ID NO |
|---|---|---|
| HCDR1 | SYSMN | 1 |
| (KABAT) | | |
| HCDR2 | SISSSSSYIYYADSVKG | 2 |
| (KABAT) | | |
| HCDR3 | RHGYSNSDAFDN | 3 |
| (KABAT) | | |
| LCDR1 | RASQGISHYLV | 4 |
| (KABAT) | | |
| LCDR2 | AASSLQS | 5 |
| (KABAT) | | |
| LCDR3 | LQHNSYPWT | 6 |
| (KABAT) | | |
| VH | EVQLVESGGGLVKPGGSLRLSCVASGFTFSSYSMNW | 7 |
| | VRQAPGKGLEWVSSISSSSSYIYYADSVKGRFTISRD | |
| | NAKNSLYLQMNSLRAEDTAVYYCARRHGYSNSDAF | |
| | DNWGQGTLVTVSS | |
| VL | DIQMTQSPSAMSASVGDRVTITCRASQGISHYLVWF | 8 |
| | QQKPGKVPKRLIYAASSLQSGVPSRFSGSGSGTEFTL | |
| | TISSLQPEDFATYYCLQHNSYPWTFGQGTKVEIK | |
| Fab HC | EVQLVESGGGLVKPGGSLRLSCVASGFTFSSYSMNW | 9 |
| | VRQAPGKGLEWVSSISSSSSYIYYADSVKGRFTISRD | |
| | NAKNSLYLQMNSLRAEDTAVYYCARRHGYSNSDAF | |
| | DNWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | RVEPKC | |
| Fab LC | DIQMTQSPSAMSASVGDRVTITCRASQGISHYLVWF | 10 |
| | QQKPGKVPKRLIYAASSLQSGVPSRFSGSGSGTEFTL | |
| | TISSLQPEDFATYYCLQHNSYPWTFGQGTKVEIKRTV | |
| | AAPSVFIFPPSDEQLKSGTASVVCLLNNFYPREAKVQ | |
| | WKVDNALQSGNSQESVTEQDSKDSTYSLSSTLTLSK | |
| | ADYEKHKVYACEVTHQGLSSPVTKSFNRGEC | |
| Fab-VHH HC | EVQLVESGGGLVKPGGSLRLSCVASGFTFSSYSMNW | 11 |
| | VRQAPGKGLEWVSSISSSSSYIYYADSVKGRFTISRD | |
| | NAKNSLYLQMNSLRAEDTAVYYCARRHGYSNSDAF | |
| | DNWGQGTLVTVSSASTKGPCVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | RVEPKCDKTHTGGGGQGGGGQGGGGQGGGGQGGG | |
| | GQEVQLLESGGGLVQPGGSLRLSCAASGRYIDETAV | |
| | AWFRQAPGKGREFVAGIGGGVDITYYADSVKGRFTI | |
| | SRDNSKNTLYLQMNSLRPEDTAVYYCGARPGRPLIT | |
| | SKVADLYPYWGQGTLVTVSSPP | |
| Fab-VHH LC | DIQMTQSPSAMSASVGDRVTITCRASQGISHYLVWF | 12 |
| | QQKPGKVPKRLIYAASSLQSGVPSRFSGSGSGTEFTL | |
| | TISSLQPEDFATYYCLQHNSYPWTFGQGTKVEIKRTV | |
| | AAPSVFIFPPSDEQLKSGTASVVCLLNNFYPREAKVQ | |
| | WKVDNALQCGNSQESVTEQDSKDSTYSLSSTLTLSK | |
| | ADYEKHKVYACEVTHQGLSSPVTKSFNRGEC | |
| hIgG4 PAA HC | EVQLVESGGGLVKPGGSLRLSCVASGFTFSSYSMNW | 13 |
| | VRQAPGKGLEWVSSISSSSSYIYYADSVKGRFTISRD | |
| | NAKNSLYLQMNSLRAEDTAVYYCARRHGYSNSDAF | |
| | DNWGQGTLVTVSSASTKGPXVFPLAPCSRSTSESTA | |
| | ALGCLVKDYFPEPVTVSWNSGALTSGVHTFPAVLQS | |
| | SGLYSLSSVVTVPSSSLGTKTYTCNVDHKPSNTKVD | |
| | KRVESKYGPPCPPCPAPEAAGGPSVFLFPPKPKDTLM | |
| | ISRTPEVTCVVVDVSQEDPEVQFNWYVDGVEVHNA | |
| | KTKPREEQFNSTYRVVSVLTVLHQDWLNGKEYKCK | |
| | VSNKGLPSSIEKTISKAKGQPREPQVYTLPPSQEEMT | |
| | KNQVSLTCLVKGFYPSDIAVEWESNGQPENNYKTTP | |
| | PVLDSDGSFFLYSRLTVDKSRWQEGNVFSCSVMHEA | |
| | LHNHYTQKSLSLSLG, wherein X is S or C. | |
| OAH1 (one arm | EVQLVESGGGLVKPGGSLRLSCVASGFTFSSYSMNW | 14 |
| heteromab) HC1 | VRQAPGKGLEWVSSISSSSSYIYYADSVKGRFTISRD | |
| (A378C) | NAKNSLYLQMNSLRAEDTAVYYCARRHGYSNSDAF | |
| | DNWGQGTLVTVSSASTKGPSVFPLAPCSRSTSESTAA | |
| | LGCLVKDYFPEPVTVSWNSGALTSGVHTFPAVLQSS | |
| | GLYSLSSVVTVPSSSLGTKTYTCNVDHKPSNTKVDK | |
| | RVESKYGPPCPPCPAPEAAGGPSVFLFPPKPKDTLMI | |
| | SRTPEVTCVVVDVSQEDPEVQFNWYVDGVEVHNAK | |
| | TKPREEQFNSTYRVVSVLTVLHQDWLNGKEYKCKV | |
| | SNKGLPSSIEKTISKAKGQPREPQVSTLPPSQEEMTKN | |
| | QVSLMCLVYGFYPSDICVEWESNGQPENNYKTTPPV | |
| | LDSDGSFFLYSVLTVDKSRWQEGNVFSCSVMHEAL | |
| | HNHYTQKSLSLSLG | |
| OAH1 HC2 | ESKYGPPCPPCPAPEAAGGPSVFLFPPKPKDTLMISRT | 15 |
| (A378C) | PEVTCVVVDVSQEDPEVQFNWYVDGVEVHNAKTKP | |
| | REEQFNSTYRVVSVLTVLHQDWLNGKEYKCKVSNK | |
| | GLPSSIEKTISKAKGQPREPQVYTLPPSQGDMTKNQV | |
| | QLTCLVKGFYPSDICVEWESNGQPENNYKTTPPVLD | |
| | SDGSFFLASRLTVDKSRWQEGNVFSCSVMHEALHN | |
| | HYTQKSLSLSLG | |
| OAH LC | DIQMTQSPSAMSASVGDRVTITCRASQGISHYLVWF | 10 |
| | QQKPGKVPKRLIYAASSLQSGVPSRFSGSGSGTEFTL | |
| | TISSLQPEDFATYYCLQHNSYPWTFGQGTKVEIKRTV | |
| | AAPSVFIFPPSDEQLKSGTASVVCLLNNFYPREAKVQ | |
| | WKVDNALQSGNSQESVTEQDSKDSTYSLSSTLTLSK | |
| | ADYEKHKVYACEVTHQGLSSPVTKSFNRGEC | |
| OAH2 HC1 | EVQLVESGGGLVKPGGSLRLSCVASGFTFSSYSMNW | 16 |
| (S124C) | VRQAPGKGLEWVSSISSSSSYIYYADSVKGRFTISRD | |
| | NAKNSLYLQMNSLRAEDTAVYYCARRHGYSNSDAF | |
| | DNWGQGTLVTVSSASTKGPCVFPLAPCSRSTSESTA | |
| | ALGCLVKDYFPEPVTVSWNSGALTSGVHTFPAVLQS | |
| | SGLYSLSSVVTVPSSSLGTKTYTCNVDHKPSNTKVD | |
| | KRVESKYGPPCPPCPAPEAAGGPSVFLFPPKPKDTLM | |
| | ISRTPEVTCVVVDVSQEDPEVQFNWYVDGVEVHNA | |
| | KTKPREEQFNSTYRVVSVLTVLHQDWLNGKEYKCK | |
| | VSNKGLPSSIEKTISKAKGQPREPQVSTLPPSQEEMTK | |
| | NQVSLMCLVYGFYPSDIAVEWESNGQPENNYKTTPP | |
| | VLDSDGSFFLYSVLTVDKSRWQEGNVFSCSVMHEA | |
| | LHNHYTQKSLSLSLG | |
| OAH2 HC2 | ESKYGPPCPPCPAPEAAGGPSVFLFPPKPKDTLMISRT | 17 |
| (S124C) | PEVTCVVVDVSQEDPEVQFNWYVDGVEVHNAKTKP | |
| | REEQFNSTYRVVSVLTVLHQDWLNGKEYKCKVSNK | |
| | GLPSSIEKTISKAKGQPREPQVYTLPPSQGDMTKNQV | |
| | QLTCLVKGFYPSDIAVEWESNGQPENNYKTTPPVLD | |
| | SDGSFFLASRLTVDKSRWQEGNVFSCSVMHEALHN | |
| | HYTQKSLSLSLG | |
| Null Arm HC | QVQLVQSGAEVKKPGSSVKVSCKASGYTFSSYAIEW | 18 |
| | VRQAPGQGLEWMGGILPGSGTINYNEKFKGRVTITA | |
| | DKSTSTAYMELSSLRSEDTAVYYCARMSSNSDQGFD | |
| | LWGQGTLVTVSSASTKGPXVFPLAPCSRSTSESTAAL | |
| | GCLVKDYFPEPVTVSWNSGALTSGVHTFPAVLQSSG | |
| | LYSLSSVVTVPSSSLGTKTYTCNVDHKPSNTKVDKR | |
| | VESKYGPPCPPCPAPEAAGGPSVFLFPPKPKDTLMIS | |
| | RTPEVTCVVVDVSQEDPEVQFNWYVDGVEVHNAKT | |
| | KPREEQFNSTYRVVSVLTVLHQDWLNGKEYKCKVS | |
| | NKGLPSSIEKTISKAKGQPREPQVYTLPPSQEEMTKN | |
| | QVSLTCLVKGFYPSDIAVEWESNGQPENNYKTTPPV | |
| | LDSDGSFLLYSKLTVDKSRWQEGNVFSCSVMHEAL | |
| | HNHYTQKSLSLSLG, wherein X is S or C. | |
| Null Arm LC | DIQMTQSPSSLSASVGDRVTITCKASQGISRFLSWFQ | 19 |
| | QKPGKAPKSLIYAVSSLVDGVPSRFSGSGSGTDFTLTI | |
| | SSLQPEDFATYYCVQYNSYPYGFGGGTKVEIKRTVA | |
| | APSVFIFPPSDEQLKSGTASVVCLLNNFYPREAKVQW | |
| | KVDNALQSGNSQESVTEQDSKDSTYSLSSTLTLSKA | |
| | DYEKHKVYACEVTHQGLSSPVTKSFNRGEC | |

TABLE 1b
Exemplary sequences of human TfR binding proteins

| Human TfR | | | | |
|---|---|---|---|---|
| binding | | | | |
| protein (TBP) | HC1 | LC1 | HC2 | LC2 |
| TBP1 | SEQ ID NO: 9 | SEQ ID NO: 10 | N/A | N/A |
| (Fab) | | | | |
| TBP2 | SEQ ID NO: 11 | SEQ ID NO: 12 | N/A | N/A |
| (Fab-VHH) | | or 10 | | |
| TBP3 | SEQ ID NO: 13 | SEQ ID NO: 10 | SEQ ID NO: 18 | SEQ ID NO: 19 |
| (Heterodimeric | | | | |
| Ab) | | | | |
| TBP4 | SEQ ID NO: 14 | SEQ ID NO: 10 | SEQ ID NO: 15 | N/A* |
| (One Arm | | | | |
| Heteromab 1, | | | | |
| A378C) | | | | |
| TBP5 | SEQ ID NO: 16 | SEQ ID NO: 10 | SEQ ID NO: 17 | N/A* |
| (One Arm | | | | |
| Heteromab 2, | | | | |
| S124C) | | | | |

In some embodiments, the monovalent human TfR binding domain is an antibody fragment, e.g., Fab, scFv, Fv, or scFab (single chain Fab). In some embodiments, the monovalent human TfR binding domain is Fab. In some embodiments, the human TfR binding domain further comprises a heavy chain constant region and/or a light chain constant region.

In some embodiments, the human TfR binding protein further comprises a half-life extender, e.g., an immunoglobulin Fc region or a VHH that binds human serum albumin (HSA).

In some embodiments, the human TfR binding protein further comprises an immunoglobulin Fc region, e.g., a modified human IgG4 Fc region, or a modified human IgG1 Fc region. In some embodiments, the human TfR binding protein further comprises a modified human IgG4 Fc region comprising proline at residue 228, and alanine at residues 234 and 235 (all residues are numbered according to the EU Index numbering, also called hIgG4PAA Fc region). In some embodiments, the human TfR binding protein further comprises a modified human IgG1 Fc region comprising alanine at residues 234, 235, and 329, serine at position 265, aspartic acid at position 436 (all residues are numbered according to the EU Index numbering, also called hIgG1 effector null or hIgG1EN Fc region).

In some embodiments, the human TfR binding protein further comprise a VHH that binds human HSA. In some embodiments, the VHH also binds mouse, rat, and/or Cynomolgus monkey albumin. An exemplary VHH that binds human HSA is shown in Table 2. In some embodiments, such a VHH comprises CDR1 comprising SEQ ID NO: 20, CDR2 comprising SEQ ID NO: 21, and CDR3 comprising SEQ ID NO: 22. In some embodiments, such a VHH comprises SEQ ID NO: 23. In some embodiments, the VHH is linked to the TfR binding domain through a peptide linker, e.g., (GGGGQ)₄ (SEQ ID NO: 24). In some embodiments, the VHH is linked to the C-terminus of the TfR binding domain.

TABLE 2
Exemplary sequences of VHH that binds human
serum albumin (HSA)

| | | SEQ |
|---|---|---|
| Region | Sequence | ID NO |
| CDR1 | ETAVA | 20 |
| (KABAT) | | |
| CDR2 | GIGGGVDITYYADSVKG | 21 |
| (KABAT) | | |
| CDR3 | RPGRPLITSKVADLYPY | 22 |
| (KABAT) | | |
| VHH full | EVQLLESGGGLVQPGGSLRLSCAASGRYIDETAV | 23 |
| length | AWFRQAPGKGREFVAGIGGGVDITYYADSVKGR | |
| | FTISRDNSKNTLYLQMNSLRPEDTAVYYCGARPG | |
| | RPLITSKVADLYPYWGQGTLVTVSSPP | |
| Optional | GGGGQGGGGQGGGGQGGGGQ | 24 |
| linker | | |

In some embodiments, the human TfR binding protein is heterodimeric antibody that comprises a first arm comprising one monovalent human TfR binding domain and a second arm that is a null arm, e.g., an arm that does not bind any known human target (e.g., an isotype arm). Heterodimeric antibodies such as heteromab, orthomab or duobody have been described in WO2014150973, WO2016118742, WO2018118616, and WO2011131746. In some embodiments, the first arm comprises any monovalent human TfR binding domain described herein. In some embodiments, the second arm is a null arm that does not bind any known human target (e.g., an isotype arm) comprises the sequences in Table 1a. In some embodiments, the second arm comprises a heavy chain (HC) and a light chain (LC), wherein the HC comprises SEQ ID NO: 18, and the LC comprises SEQ ID NO: 19.

In some embodiments, the human TfR binding protein comprises heterodimeric mutations. In some embodiments, the human TfR binding protein comprises a modified Fc region comprising a first Fc CH3 domain comprising serine at residue 349, methionine at residue 366, tyrosine at residue 370, and valine at residue 409, and a second Fc CH3 domain comprising glycine at residue 356, aspartic acid at residue 357, glutamine at residue 364 and alanine at residue 407 (all residues are numbered according to the EU Index numbering). In some embodiments, the human TfR binding protein comprises a modified Fc region comprising a first Fc CH3 domain comprising leucine at residue 405, and a second Fc CH3 domain comprising arginine at residue 409 (all residues are numbered according to the EU Index numbering).

In some embodiments, the human TfR binding protein comprises one or more native cysteine residues, which can be used for conjugation. For example, in some embodiments, the human TfR binding protein comprises a native cysteine at position 220 of the light chain and/or a native cysteine at position 226 of the heavy chain, which can be used for conjugation (all residues according to the EU Index numbering).

In some embodiments, the human TfR binding protein comprises engineered cysteine residues for conjugation. The approach of including engineered cysteines as a means for conjugation has been described in WO 2018/232088. In some embodiments, the human TfR binding protein comprises a heavy chain comprising one or more cysteines at the following residues: 124, 157, 162, 262, 373, 375, 378, 397, 415 (all residues according to the EU Index numbering). In some embodiments, the human TfR binding protein comprises a light chain (e.g., a kappa light chain) comprising one or more cysteines at the following residues: 156, 171, 191, 193, 202, 208 (all residues according to the EU Index numbering). In some embodiments, the human TfR binding protein comprises a heavy chain constant region comprising cysteine at residue 124 (according to the EU Index numbering). In some embodiments, the human TfR binding protein comprises a light chain constant region comprising cysteine at residue 156 (according to the EU Index numbering). In some embodiments, the human TfR binding protein comprises an immunoglobulin Fc region comprising cysteine at residue 378 (according to the EU Index numbering).

In some embodiments, the human TfR binding protein is any one of the human TfR binding proteins in Table 1b, e.g., TBP1, TBP2, TBP3, TBP4, TBP5.

In some embodiments, the human TfR binding protein has a Fab format, e.g., TBP1. In some embodiments, the human TfR binding protein comprises one HC and one LC, and wherein the HC comprises SEQ ID NO: 9 and the LC comprises SEQ ID NO: 10.

In some embodiments, the human TfR binding protein has a Fab-VHH format, e.g., TBP2. In some embodiments, the human TfR binding proteins comprises one HC and one LC, wherein the HC comprises SEQ ID NO: 11 and the LC comprises SEQ ID NO: 12 or 10.

In some embodiments, the human TfR binding protein has a heterodimeric antibody format, e.g., TBP3. In some embodiments, the human TfR binding protein comprises two heavy chains HC1 and HC2 and two light chains LC1 and LC2, wherein HC1 comprises SEQ ID NO: 13, LC1 comprises SEQ ID NO: 10, HC2 comprises SEQ ID NO: 18, and LC2 comprises SEQ ID NO: 19.

In some embodiments, the human TfR binding protein has a one arm heteromab format, e.g., TBP4 or TBP5. In some embodiments, the human TfR binding protein comprises two heavy chains HC1 and HC2 and one light chain LC1, wherein HC1 comprises SEQ ID NO: 14, LC1 comprises SEQ ID NO: 10, HC2 comprises SEQ ID NO: 15. In some embodiments, provided herein are human TfR binding proteins comprise two heavy chains HC1 and HC2 and one light chain LC1, wherein HC1 comprises SEQ ID NO: 16, LC1 comprises SEQ ID NO: 10, HC2 comprises SEQ ID NO: 17.

The human TfR binding proteins described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the TfR binding proteins) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of the TfR binding proteins described herein. According to some embodiments, a host cell may be stably or transiently transfected, transformed, transduced or infected with an expression vector expressing HC polypeptides and an expression vector expressing LC polypeptides of the TfR binding proteins described herein. In some embodiments, a host cell may be stably or transiently transfected, transformed, transduced or infected with an expression vector expressing HC and LC polypeptides of the TfR binding proteins described herein. The TfR binding proteins may be produced in mammalian cells such as CHO, NS0, HEK293 or COS cells according to techniques well known in the art.

Medium, into which the TfR binding proteins has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

### Mouse TfR Binding Proteins

Some APP RNAi agents used in the Examples below comprise a protein comprising one monovalent mouse TfR binding domain (“mouse TfR binding proteins” or mTBP). Exemplary sequences of mouse TfR binding proteins are provided in Table 3. Such APP RNAi agents comprising a mouse TfR binding protein can serve as surrogate molecules in mouse models for APP RNAi agents comprising a human TfR binding protein.

TABLE 3
Exemplary sequences of mouse TfR binding protein (mTBP1)

| Region | Sequence | SEQ ID NO |
|---|---|---|
| HCDR1 | GSYWIC | 25 |
| (KABAT) | | |
| HCDR2 | CIYSTSGGRTYYASWVKG | 26 |
| (KABAT) | | |
| HCDR3 | GDDSISDAYFDL | 27 |
| (KABAT) | | |
| LCDR1 | QSSQSVYNNNRLA | 28 |
| (KABAT) | | |
| LCDR2 | DASTLAS | 29 |
| (KABAT) | | |
| LCDR3 | QGTYFSSGWSWA | 30 |
| (KABAT) | | |
| VH | QSLEESGGDLVKPEGSLTLTCTASGFSFSGSYWI | 31 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSS | |
| VL | ALDMTQTASPVSAAVGGTVTINCQSSQSVYNN | 32 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVK | |
| HC1 | QSLEESGGDLVKPEGSLTLTCTASGFSFSGSYWI | 33 |
| | CWVRQAPGKGLEWIGCIYSTSGGRTYYASWVK | |
| | GRFTISKTSSTTVTLQMTSLTAADTATYFCARG | |
| | DDSISDAYFDLWGPGTLVTVSSASTKGPCVFPL | |
| | APCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPP | |
| | VLDSDGSFFLYSRLTVDKSRWQEGNVFSCSVM | |
| | HEALHNHYTQKSLSLSLG | |
| LC1 | ALDMTQTASPVSAAVGGTVTINCQSSQSVYNN | 34 |
| | NRLAWYQQKPGQPPKLLIYDASTLASGVPSRFK | |
| | GSGSGTQFTLTISGVQSDDSATYYCQGTYFSSG | |
| | WSWAFGGGTEVVVKRTVAAPSVFIFPPSDEQLK | |
| | SGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Null Arm HC | QVQLVQSGAEVKKPGSSVKVSCKASGYTFSSY | 18 |
| (HC2) | AIEWVRQAPGQGLEWMGGILPGSGTINYNEKFK | |
| | GRVTITADKSTSTAYMELSSLRSEDTAVYYCAR | |
| | MSSNSDQGFDLWGQGTLVTVSSASTKGPXVFPL | |
| | APCSRSTSESTAALGCLVKDYFPEPVTVSWNSG | |
| | ALTSGVHTFPAVLQSSGLYSLSSVVTVPSSSLGT | |
| | KTYTCNVDHKPSNTKVDKRVESKYGPPCPPCPA | |
| | PEAAGGPSVFLFPPKPKDTLMISRTPEVTCVVVD | |
| | VSQEDPEVQFNWYVDGVEVHNAKTKPREEQFN | |
| | STYRVVSVLTVLHQDWLNGKEYKCKVSNKGLP | |
| | SSIEKTISKAKGQPREPQVYTLPPSQEEMTKNQV | |
| | SLTCLVKGFYPSDIAVEWESNGQPENNYKTTPP | |
| | VLDSDGSFLLYSKLTVDKSRWQEGNVFSCSVM | |
| | HEALHNHYTQKSLSLSLG, wherein X is S or C. | |
| Null Arm LC | DIQMTQSPSSLSASVGDRVTITCKASQGISRFLS | 19 |
| (LC2) | WFQQKPGKAPKSLIYAVSSLVDGVPSRFSGSGS | |
| | GTDFTLTISSLQPEDFATYYCVQYNSYPYGFGG | |
| | GTKVEIKRTVAAPSVFIFPPSDEQLKSGTASVVC | |
| | LLNNFYPREAKVQWKVDNALQSGNSQESVTEQ | |
| | DSKDSTYSLSSTLTLSKADYEKHKVYACEVTHQ | |
| | GLSSPVTKSFNRGEC | |

### Linker

In some embodiments, the APP RNAi agents described herein comprises a linker that links the human TfR binding protein to the dsRNA. In some embodiments, the linker is a Mal-Tet-TCO linker, SMCC linker, or GDM linker (structures of these linkers shown in Table 4). In some embodiments, the linker is a SMCC linker.

TABLE 4
Exemplary linker structures

| Linker | Structure |
|---|---|
| 1 | SMCC linker 1 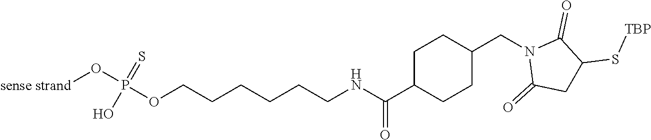 |
| 2 | SMCC linker 2 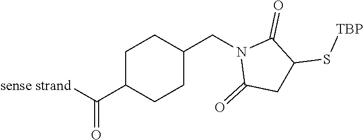 |
| 3 | Hydrolyzed ring open form of SMCC linker 1 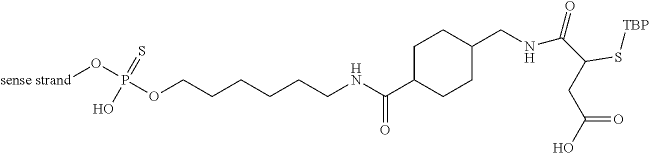 |
| 4 | Hydrolyzed ring open form of SMCC linker 2 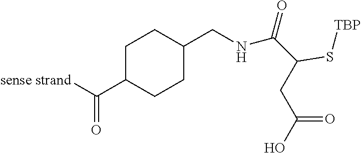 |
| 5 | Mal-Tet-TCO linker1 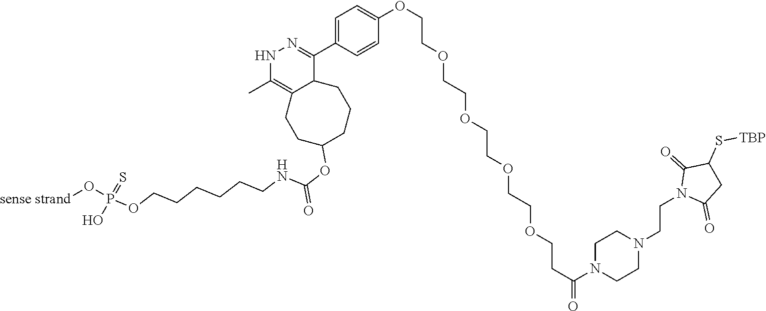 |
| 6 | Mal-Tet-TCO linker2 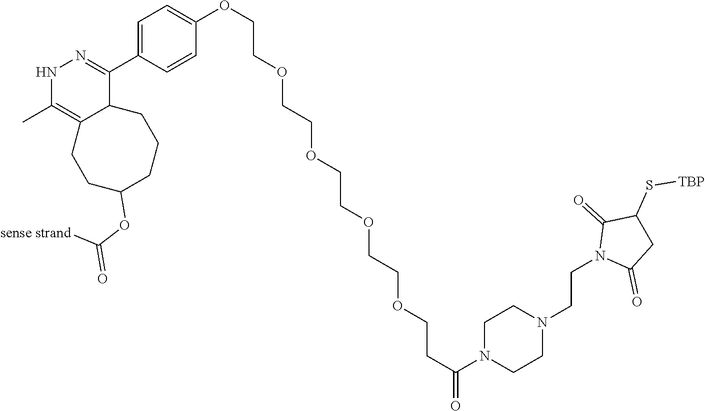 |
| 7 | GDM linker1 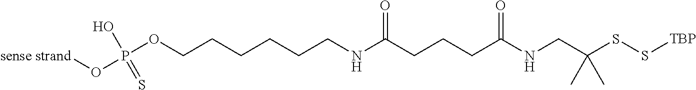 |
| 8 | GDM linker2 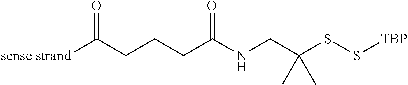 |

dsRNA

The APP RNAi agents described herein comprise a double stranded RNA (dsRNA) comprising a sense stand and an antisense strand, and wherein the antisense strand is complementary to APP mRNA. After the antisense strand of the dsRNA is incorporated into the RNA-induced silencing complex (RISC), the RISC can bind and degrade target APP mRNA.

In some embodiments, the sense strand and the antisense strand of the dsRNA are each 15-30 nucleotides in length, e.g., 20-25 nucleotides in length. In some embodiments, the dsRNA has a sense strand of 21 nucleotides and an antisense strand of 23 nucleotides. In some embodiments, the sense strand and antisense strand of the dsRNA may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

Exemplary unmodified sense strand and antisense strand sequences of dsRNA targeting human APP mRNA are provided in Table 5a and 5b.

TABLE 5a
Unmodified Nucleic Acid Sequences of dsRNA targeting 3′ UTR of human APP
mRNA

| | | | | | Start |
|---|---|---|---|---|---|
| | | | | | position of |
| | | | | | target |
| | | | | | region on |
| | | | | | human |
| | | SEQ | | SEQ | APP mRNA |
| dsRNA | Sense Strand | ID | Antisense Strand | ID | transcript |
| No. | (5′ to 3′) | NO | (5′ to 3′) | NO | NM_000484.4 |
| 1 | AGCAAAACCAUUGC | 35 | UAGUGAAGCAAUGGUUUU | 36 | 2495 |
| | UUCACUA | | GCUGU | | |
| 2 | CAGCAAAACCAUUG | 37 | UGUGAAGCAAUGGUUUUG | 38 | 2494 |
| | CUUCACA | | CUGUC | | |
| 3 | GAUGCCUGAACUUG | 39 | UUUAAUUCAAGUUCAGGC | 40 | 2614 |
| | AAUUAAA | | AUCUA | | |
| 4 | CUGUAACACAAGUA | 41 | UGGCAUCUACUUGUGUUA | 42 | 2600 |
| | GAUGCCA | | CAGCA | | |
| 5 | CCUGAUCACUAUGC | 43 | UUAAAAUGCAUAGUGAUC | 44 | 2913 |
| | AUUUUAA | | AGGAA | | |
| 6 | UGACAGCUGUGCUG | 45 | UGUGUUACAGCACAGCUG | 46 | 2591 |
| | UAACACA | | UCAAA | | |
| 7 | CAUGAAUAGAUUCU | 47 | UCAGGAGAGAAUCUAUUC | 48 | 2740 |
| | CUCCUGA | | AUGCA | | |
| 8 | GUACAGAAUCAUUG | 49 | UCAUAAGCAAUGAUUCUG | 50 | 3259 |
| | CUUAUGA | | UACAA | | |
| 9 | UGUCCACGUAUCUU | 51 | UGACCCAAAGAUACGUGG | 52 | 3120 |
| | UGGGUCA | | ACAAA | | |
| 10 | GUCCACGUAUCUUU | 53 | UAGACCCAAAGAUACGUG | 54 | 3121 |
| | GGGUCUA | | GACAA | | |
| 11 | GAUAUAGGAAUUAA | 55 | UAUCCUCUUAAUUCCUAU | 56 | 3028 |
| | GAGGAUA | | AUCAC | | |
| 12 | ACAGAUUGCUGCUU | 57 | UUAGCAGAAGCAGCAAUC | 58 | 3001 |
| | CUGCUAA | | UGUAC | | |
| 13 | AGCAAAACC(n)UUGC | 184 | UAGUGAAGCAAUGGUUUU | 36 | 2495 |
| | UUCACUA, wherein n is | | GCUGU | | |
| | an abasic moiety. | | | | |
| 14 | CAGCAAAAC(n)AUUG | 188 | UGUGAAGCAAUGGUUUUG | 38 | 2494 |
| | CUUCACA, wherein n is | | CUGUC | | |
| | an abasic moiety. | | | | |
| 106 | AGCAAAACCAUUGC | 35 | UAGUGAAGCAAUGGUUUU | 214 | 2495 |
| | UUCACUA | | GCUG | | |

Table 5b. Unmodified Nucleic Acid Sequences of dsRNA Targeting the Coding Sequence of Human APP mRNA

| | | | | | Start |
|---|---|---|---|---|---|
| | | | | | position |
| | | | | | of target |
| | | | | | region on |
| | | | | | human |
| | | SEQ | | SEQ | APP mRNA |
| dsRNA | Sense Strand | ID | Antisense Strand | ID | transcript |
| No. | (5′ to 3′) | NO | (5′ to 3′) | NO | NM_000484.4 |
| 15 | CCAAGAUGCAGCAGA | 59 | UGCCGUUCUGCUGCAUCU | 60 | 2399 |
| | ACGGCA | | UGGAC | | |
| 16 | AGGAAGCAGCCAACG | 61 | UUCUCUCGUUGGCUGCUU | 62 | 1481 |
| | AGAGAA | | CCUGU | | |
| 17 | CUUUGAGCAGAUGCA | 63 | UAGUUCUGCAUCUGCUCA | 64 | 2442 |
| | GAACUA | | AAGAA | | |
| 18 | GGCAGUUAUCCAGCA | 65 | UGGAAAUGCUGGAUAACU | 66 | 1437 |
| | UUUCCA | | GCCUU | | |
| 19 | UCCAACCUACAAGUU | 67 | UCAAAGAACUUGUAGGUU | 68 | 2427 |
| | CUUUGA | | GGAUU | | |
| 20 | CCAACCUACAAGUUC | 69 | UUCAAAGAACUUGUAGGU | 70 | 2428 |
| | UUUGAA | | UGGAU | | |
| 21 | CUGAAGAAGAAACAG | 71 | UGUGUACUGUUUCUUCUU | 72 | 2317 |
| | UACACA | | CAGCA | | |
| 22 | GACAAAGUAGUAGAA | 73 | UGCUACUUCUACUACUUU | 74 | 817 |
| | GUAGCA | | GUCUU | | |
| 23 | AAGUUCUUUGAGCAG | 75 | UUGCAUCUGCUCAAAGAA | 76 | 2437 |
| | AUGCAA | | CUUGU | | |
| 24 | AGGCAGUUAUCCAGC | 77 | UGAAAUGCUGGAUAACUG | 78 | 1436 |
| | AUUUCA | | CCUUC | | |
| 25 | AGCACCGAGAGAGAA | 79 | UGGACAUUCUCUCUCGGU | 80 | 1352 |
| | UGUCCA | | GCUUG | | |
| 26 | CCGGUCCCAGGUUAU | 81 | UGUGUCAUAACCUGGGAC | 82 | 1728 |
| | GACACA | | CGGAU | | |
| 27 | AACCAGUGACCAUCC | 83 | UGUUCUGGAUGGUCACUG | 84 | 419 |
| | AGAACA | | GUUGG | | |
| 28 | ACCGAGGACUGACCA | 85 | UUCGAGUGGUCAGUCCUC | 86 | 2090 |
| | CUCGAA | | GGUCG | | |
| 29 | CACACCGUCGCCAAA | 87 | UGUCUCUUUGGCGACGGU | 88 | 601 |
| | GAGACA | | GUGCC | | |
| 30 | GCCAAGCACCGAGAG | 89 | UAUUCUCUCUCGGUGCUU | 90 | 1348 |
| | AGAAUA | | GGCCU | | |
| 31 | GAUCCGGUCCCAGGU | 91 | UUCAUAACCUGGGACCGG | 92 | 1725 |
| | UAUGAA | | AUCUG | | |
| 32 | AAGCACCGAGAGAGA | 93 | UGACAUUCUCUCUCGGUG | 94 | 1351 |
| | AUGUCA | | CUUGG | | |
| 33 | CCAUCCAGAACUGGU | 95 | UCUUGCACCAGUUCUGGA | 96 | 428 |
| | GCAAGA | | UGGUC | | |
| 34 | GUCCAAGAUGCAGCA | 97 | UCGUUCUGCUGCAUCUUG | 98 | 2397 |
| | GAACGA | | GACAG | | |
| 35 | CAGGUCAUGAGAGAA | 99 | UUCCCAUUCUCUCAUGACC | 100 | 1372 |
| | UGGGAA | | UGGG | | |

In some embodiments, the sense strand and the antisense strand of the dsRNA comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40;
(d) the sense strand comprises SEQ ID NO: 41, and the antisense strand comprises SEQ ID NO: 42;
(e) the sense strand comprises SEQ ID NO: 43, and the antisense strand comprises SEQ ID NO: 44;
(f) the sense strand comprises SEQ ID NO: 45, and the antisense strand comprises SEQ ID NO: 46;
(g) the sense strand comprises SEQ ID NO: 47, and the antisense strand comprises SEQ ID NO: 48;
(h) the sense strand comprises SEQ ID NO: 49, and the antisense strand comprises SEQ ID NO: 50;
(i) the sense strand comprises SEQ ID NO: 51, and the antisense strand comprises SEQ ID NO: 52;
(j) the sense strand comprises SEQ ID NO: 53, and the antisense strand comprises SEQ ID NO: 54;
(k) the sense strand comprises SEQ ID NO: 55, and the antisense strand comprises SEQ ID NO: 56;
(l) the sense strand comprises SEQ ID NO: 57, and the antisense strand comprises SEQ ID NO: 58;
(m) the sense strand comprises SEQ ID NO: 59, and the antisense strand comprises SEQ ID NO: 60;
(n) the sense strand comprises SEQ ID NO: 61, and the antisense strand comprises SEQ ID NO: 62;
(o) the sense strand comprises SEQ ID NO: 63, and the antisense strand comprises SEQ ID NO: 64;
(p) the sense strand comprises SEQ ID NO: 65, and the antisense strand comprises SEQ ID NO: 66;
(q) the sense strand comprises SEQ ID NO: 67, and the antisense strand comprises SEQ ID NO: 68;
(r) the sense strand comprises SEQ ID NO: 69, and the antisense strand comprises SEQ ID NO: 70;
(s) the sense strand comprises SEQ ID NO: 71, and the antisense strand comprises SEQ ID NO: 72;
(t) the sense strand comprises SEQ ID NO: 73, and the antisense strand comprises SEQ ID NO: 74;
(u) the sense strand comprises SEQ ID NO: 75, and the antisense strand comprises SEQ ID NO: 76;
(v) the sense strand comprises SEQ ID NO: 77, and the antisense strand comprises SEQ ID NO: 78;
(w) the sense strand comprises SEQ ID NO: 79, and the antisense strand comprises SEQ ID NO: 80;
(x) the sense strand comprises SEQ ID NO: 81, and the antisense strand comprises SEQ ID NO: 82;
(y) the sense strand comprises SEQ ID NO: 83, and the antisense strand comprises SEQ ID NO: 84;
(z) the sense strand comprises SEQ ID NO: 85, and the antisense strand comprises SEQ ID NO: 86;
(aa) the sense strand comprises SEQ ID NO: 87, and the antisense strand comprises SEQ ID NO: 88;
(bb) the sense strand comprises SEQ ID NO: 89, and the antisense strand comprises SEQ ID NO: 90;
(cc) the sense strand comprises SEQ ID NO: 91, and the antisense strand comprises SEQ ID NO: 92;
(dd) the sense strand comprises SEQ ID NO: 93, and the antisense strand comprises SEQ ID NO: 94;
(ee) the sense strand comprises SEQ ID NO: 95, and the antisense strand comprises SEQ ID NO: 96;
(ff) the sense strand comprises SEQ ID NO: 97, and the antisense strand comprises SEQ ID NO: 98;
(gg) the sense strand comprises SEQ ID NO: 99, and the antisense strand comprises SEQ ID NO: 100;
(hh) the sense strand comprises SEQ ID NO: 184, and the antisense strand comprises SEQ ID NO: 36;
(ii) the sense strand comprises SEQ ID NO: 188, and the antisense strand comprises SEQ ID NO: 38; and
(jj) the sense strand comprises SEQ ID NO: 35, and the antisense strand comprises SEQ ID NO: 214;
   wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 35, and the antisense strand comprises SEQ ID NO: 36. In some embodiments, the sense strand comprises SEQ ID NO: 35, and the antisense strand comprises SEQ ID NO: 214. In some embodiments, the sense strand comprises SEQ ID NO: 37, and the antisense strand comprises SEQ ID NO: 38.

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl.

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, at least one nucleotide of the sense strand is an unmodified RNA nucleotide. In some embodiments, each nucleotide of the antisense strand is a modified nucleotide. In some embodiments, the modified nucleotide is a 2′-fluoro modified nucleotide, 2′-O-methyl modified nucleotide, 2′ deoxy nucleotide (DNA), or 2′-O-alkyl modified nucleotide. In some embodiments, each nucleotide of the sense strand and the antisense strand is independently a modified nucleotide, e.g., a 2′-fluoro modified nucleotide, 2′-O-methyl modified nucleotide, 2′ deoxy nucleotide (DNA), or 2′-O-alkyl modified nucleotide. In some embodiments, at least one nucleotide of the sense strand is 2′ deoxy nucleotide (DNA).

In some embodiments, the sense strand has four 2′-fluoro modified nucleotides, e.g., at positions 7, 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, at least one nucleotide of the sense strand is an unmodified RNA nucleotide. In some embodiments, at least one nucleotide of the sense strand is 2′ deoxy nucleotide (DNA). In some embodiments, the other nucleotides of the sense strand are 2′-O-methyl modified nucleotides.

In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 6. In some embodiments, the inverted basic moiety or abasic moiety increases stability of the sense strand or the antisense strand. In some embodiments, the sense strand comprises an inverted abasic moiety. In some embodiments, the antisense strand comprises an inverted abasic moiety.

TABLE 6
Abasic or inverted abasic (iAb) moieties

| | Structure |
|---|---|
| 1 (abasic) | 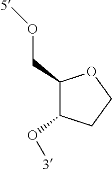 |
| 2 (iAb) | 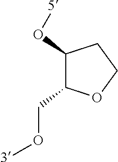 |

“5′” and “3′” indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human APP mRNA are provided in Table 7a and 7b.

In some embodiments, the dsRNA comprises a sense strand that comprises a sequence that has 1, 2, or 3 differences from a sense stand sequence in Table 7a or 7b. In some embodiments, the dsRNA comprises an antisense strand that comprises a sequence that has 1, 2, or 3 differences from an antisense stand sequence in Table 7a or 7b.

TABLE 7a
Modified Nucleic Acid Sequences of dsRNA targeting 3′ UTR of human APP
mRNA

| | | SEQ |
|---|---|---|
| dsRNA | | ID |
| No. | Oligo Sequence 5′ to 3′ | NO |
| 36 | mA*mG*mCmAmAmAfAmCfCfAfUmUmGmCmUmUmCmAmC*mU*mA | 101 |
| | mU*fA*mGmUmGfAmAmGmCmAmAmUmGfGmUfUmUmUmGmCmU*mG*mU | 102 |
| 37 | mC*mA*mGmCmAmAfAmAfCfCfAmUmUmGmCmUmUmCmA*mC*mA | 103 |
| | mU*fG*mUmGmAfAmGmCmAmAmUmGmGfUmUfUmUmGmCmUmG*mU*mC | 104 |
| 38 | mG*mA*mUmGmCmCfUmGfAfAfCmUmUmGmAmAmUmUmA*mA*mA | 105 |
| | mU*fU*mUmAmAfUmUmCmAmAmGmUmUfCmAfGmGmCmAmUmC*mU*mA | 106 |
| 39 | mC*mU*mGmUmAmAfCmAfCfAfAmGmUmAmGmAmUmGmC*mC*mA | 107 |
| | mU*fG*mGmCmAfUmCmUmAmCmUmUmGfUmGfUmUmAmCmAmG*mC*mA | 108 |
| 40 | mC*mC*mUmGmAmUfCmAfCfUfAmUmGmCmAmUmUmUmU*mA*mA | 109 |
| | mU*fU*mAmAmAfAmUmGmCmAmUmAmGfUmGfAmUmCmAmGmG*mA*mA | 110 |
| 41 | mU*mG*mAmCmAmGfCmUfGfUfGmCmUmGmUmAmAmCmA*mC*mA | 111 |
| | mU*fG*mUmGmUfUmAmCmAmGmCmAmCfAmGfCmUmGmUmCmA*mA*mA | 112 |
| 42 | mC*mA*mUmGmAmAfUmAfGfAfUmUmCmUmCmUmCmCmU*mG*mA | 113 |
| | mU*fC*mAmGmGfAmGmAmGmAmAmUmCfUmAfUmUmCmAmUmG*mC*mA | 114 |
| 43 | mG*mU*mAmCmAmGfAmAfUfCfAmUmUmGmCmUmUmAmU*mG*mA | 115 |
| | mU*fC*mAmUmAfAmGmCmAmAmUmGmAfUmUfCmUmGmUmAmC*mA*mA | 116 |
| 44 | mU*mG*mUmCmCmAfCmGfUfAfUmCmUmUmUmGmGmGmU*mC*mA | 117 |
| | mU*fG*mAmCfCmCfAmAmAmGmAmUmAfCmGfUmGmGmAmCmA*mA*mA | 118 |
| 45 | mG*mU*mCmCmAmCfGmUfAfUfCmUmUmUmGmGmGmUmC*mU*mA | 119 |
| | mU*fA*mGmAfCmCfCmAmAmAmGmAmUfAmCfGmUmGmGmAmC*mA*mA | 120 |
| 46 | mG*mA*mUmAmUmAfGmGfAfAfUmUmAmAmGmAmGmGmA*mU*mA | 121 |
| | mU*fA*mUmCfCmUfCmUmUmAmAmUmUfCmCfUmAmUmAmUmC*mA*mC | 122 |
| 47 | mA*mC*mAmGmAmUfUmGfCfUfGmCmUmUmCmUmGmCmU*mA*mA | 123 |
| | mU*fU*mAmGfCmAfGmAmAmGmCmAmGfCmAfAmUmCmUmGmU*mA*mC | 124 |
| 48 | mA*mG*mCmAmAmAmAmCfCfAfUmUmGmCmUmUmCmAmC*mU*mA | 172 |
| | mU*fA*mGmUfGmAfAmGmCmAmAmUmGfGmUfUmUmUmGmCmU*mG*mU | 173 |
| 49 | mA*mG*mCmAmAmAfAmCfCfAfUmUmGmCmUmUmCmAmC*mU*mA | 101 |
| | mU*fA*mGmUfGmAfAmGmCmAmAmUmGfGmUfUmUmUmGmCmU*mG*mU | 173 |
| 50 | mC*mA*mGmCmAmAmAmAfCfCfAmUmUmGmCmUmUmCmA*mC*mA | 174 |
| | mU*fG*mUmGfAmAfGmCmAmAmUmGmGfUmUfUmUmGmCmUmG*mU*mC | 175 |
| 51 | mC*mA*mGmCmAmAfAmAfCfCfAmUmUmGmCmUmUmCmA*mC*mA | 103 |
| | mU*fG*mUmGfAmAfGmCmAmAmUmGmGfUmUfUmUmGmCmUmG*mU*mC | 175 |
| 52 | mA*mG*mCmAmAmAfAmCfCfAfUmUmGmCmUmUmCmAmC*mU*mA | 101 |
| | mU*fA*mGmUfGmAmAmGmCmAmAmUmGfGmUfUmUmUmGmCmU*mG*mU | 176 |
| 53 | mA*mG*mCmAmAmAfAmCfCfAfUmUmGmCmUmUmCmAmC*mU*mA | 101 |
| | mU*fA*mGmUfGmAmAfGmCmAmAmUmGfGmUfUmUmUmGmCmU*mG*mU | 177 |
| 54 | mA*mG*mCmAmAmAfAmCfCfAfUmUmGmCmUmUmCmAmC*mU*mA | 101 |
| | mU*fA*mGfUmGmAmAmGmCmAmAmUmGfGmUfUmUmUmGmCmU*mG*mU | 178 |
| 55 | mA*mG*mCmAmAmAfAmCfCfAfUmUmGmCmUmUmCmAmC*mU*mA | 101 |
| | mU*fA*mGmUmGmAmAmGmCmAmAmUmGfGmUfUmUmUmGmCmU*mG*mU | 179 |
| 56 | mA*mG*mCmAmAmAfAmCfCfAfUmUmGmCmUmUmCmAmC*mU*mA | 101 |
| | mU*fA*fGmUmGmAfAmGmCmAmAmUmGfGmUfUmUmUmGmCmU*mG*mU | 180 |
| 57 | mA*mG*mCmAmAmAAmCfCfAfUmUmGmCmUmUmCmAmC*mU*mA | 181 |
| | mU*fA*mGmUfGmAfAmGmCmAmAmUmGfGmUfUmUmUmGmCmU*mG*mU | 173 |
| 58 | mA*mG*mCmAmAmAfAmCfCfAfUmUmGmCmUmUmCmAmC*mU*mA | 101 |
| | mU*fA*mGfUmGfAfAmGfCmAmAmUmGfGmUfUmUmUmGmCmU*mG*mU | 182 |
| 59 | mA*mG*mCmAmAmAfAmCfC(n)fUmUmGmCmUmUmCmAmC*mU*mA | 183 |
| | mU*fA*mGmUmGfAmAmGmCmAmAmUmGfGmUfUmUmUmGmCmU*mG*mU | 102 |
| 60 | mA*mG*mCmAmAmAfAmCfCfAfUmUmGmCmUmUmCmAmC*mU*mA | 101 |
| | mU*fA*mGmUfGfAfAfGmCmAmAmUmGfGmUfUmUmUmGmCmU*mG*mU | 185 |
| 61 | mA*mG*mCmAmAmAAmCfCfAfUmUmGmCmUmUmCmAmC*mU*mA | 181 |
| | mU*fA*mGmUmGfAmAmGmCmAmAmUmGfGmUfUmUmUmGmCmU*mG*mU | 102 |
| 62 | mA*mG*mCmAmAmAfAmCfCdAfUmUmGmCmUmUmCmAmC*mU*mA | 186 |
| | mU*fA*mGmUmGfAmAmGmCmAmAmUmGfGmUfUmUmUmGmCmU*mG*mU | 102 |
| 63 | mA*mG*mCmAmAmAmAmCfCfAfUmUmGmCmUmUmCmAmC*mU*mA | 172 |
| | mU*fA*mGmUfGmAmAfGmCmAmAmUmGfGmUfUmUmUmGmCmU*mG*mU | 177 |
| 64 | mC*mA*mGmCmAmAfAmAfC(n)fAmUmUmGmCmUmUmCmA*mC*mA | 187 |
| | mU*fG*mUmGmAfAmGmCmAmAmUmGmGfUmUfUmUmGmCmUmG*mU*mC | 104 |
| 65 | mC*mA*mGmCmAmAfAmAfCfCfAmUmUmGmCmUmUmCmA*mC*mA | 103 |
| | mU*fG*mUmGfAmAmGfCmAmAmUmGmGfUmUfUmUmGmCmUmG*mU*mC | 189 |
| 66 | mC*mA*mGmCmAmAfAmAfCfCfAmUmUmGmCmUmUmCmA*mC*mA | 103 |
| | mU*fG*mUfGmAmAmGmCmAmAmUmGmGfUmUfUmUmGmCmUmG*mU*mC | 190 |
| 67 | mC*mA*mGmCmAmAfAmAfCfCfAmUmUmGmCmUmUmCmA*mC*mA | 103 |
| | mU*fG*mUmGmAmAmGmCmAmAmUmGmGfUmUfUmUmGmCmUmG*mU*mC | 191 |
| 68 | mC*mA*mGmCmAmAfAmAfCfCfAmUmUmGmCmUmUmCmA*mC*mA | 103 |
| | mU*fG*fUmGmAmAfGmCmAmAmUmGmGfUmUfUmUmGmCmUmG*mU*mC | 192 |
| 69 | mC*mA*mGmCmAmAAmAfCfCfAmUmUmGmCmUmUmCmA*mC*mA | 193 |
| | mU*fG*mUmGfAmAfGmCmAmAmUmGmGfUmUfUmUmGmCmUmG*mU*mC | 175 |
| 70 | mC*mA*mGmCmAmAfAmAfCfCfAmUmUmGmCmUmUmCmA*mC*mA | 103 |
| | mU*fG*mUfGmAfAfGmCfAmAmUmGmGfUmUfUmUmGmCmUmG*mU*mC | 194 |
| 71 | mC*mA*mGmCmAmAfAmAfCfCfAmUmUmGmCmUmUmCmA*mC*mA | 103 |
| | mU*fG*mUmGfAmAmGmCmAmAmUmGmGfUmUfUmUmGmCmUmG*mU*mC | 195 |
| 72 | mC*mA*mGmCmAmAfAmAfCfCfAmUmUmGmCmUmUmCmA*mC*mA | 103 |
| | mU*fG*mUmGfAfAfGfCmAmAmUmGmGfUmUfUmUmGmCmUmG*mU*mC | 196 |
| 73 | mC*mA*mGmCmAmAAmAfCfCfAmUmUmGmCmUmUmCmA*mC*mA | 193 |
| | mU*fG*mUmGmAfAmGmCmAmAmUmGmGfUmUfUmUmGmCmUmG*mU*mC | 104 |
| 74 | mC*mA*mGmCmAmAfAmAfCdCfAmUmUmGmCmUmUmCmA*mC*mA | 197 |
| | mU*fG*mUmGmAfAmGmCmAmAmUmGmGfUmUfUmUmGmCmUmG*mU*mC | 104 |
| 75 | mG*mA*mUmGmCmCmUmGfAfAfCmUmUmGmAmAmUmUmA*mA*mA | 198 |
| | mU*fU*mUmAfAmUfUmCmAmAmGmUmUfCmAfGmGmCmAmUmC*mU*mA | 199 |
| 76 | mC*mU*mGmUmAmAmCmAfCfAfAmGmUmAmGmAmUmGmC*mC*mA | 200 |
| | mU*fG*mGmCfAmUfCmUmAmCmUmUmGfUmGfUmUmAmCmAmG*mC*mA | 201 |
| 77 | mC*mC*mUmGmAmUmCmAfCfUfAmUmGmCmAmUmUmUmU*mA*mA | 202 |
| | mU*fU*mAmAfAmAfUmGmCmAmUmAmGfUmGfAmUmCmAmGmG*mA*mA | 203 |
| 78 | mU*mG*mAmCmAmGmCmUfGfUfGmCmUmGmUmAmAmCmA*mC*mA | 204 |
| | mU*fG*mUmGfUmUfAmCmAmGmCmAmCfAmGfCmUmGmUmCmA*mA*mA | 205 |
| 79 | mC*mA*mUmGmAmAmUmAfGfAfUmUmCmUmCmUmCmCmU*mG*mA | 206 |
| | mU*fC*mAmGfGmAfGmAmGmAmAmUmCfUmAfUmUmCmAmUmG*mC*mA | 207 |
| 80 | mG*mU*mAmCmAmGmAmAfUfCfAmUmUmGmCmUmUmAmU*mG*mA | 208 |
| | mU*fC*mAmUfAmAfGmCmAmAmUmGmAfUmUfCmUmGmUmAmC*mA*mA | 209 |
| 81 | mU*mG*mUmCmCmAmCmGfUfAfUmCmUmUmUmGmGmGmU*mC*mA | 210 |
| | mU*fG*mAmCfCmCfAmAmAmGmAmUmAfCmGfUmGmGmAmCmA*mA*mA | 118 |
| 82 | mG*mU*mCmCmAmCmGmUfAfUfCmUmUmUmGmGmGmUmC*mU*mA | 211 |
| | mU*fA*mGmAfCmCfCmAmAmAmGmAmUfAmCfGmUmGmGmAmC*mA*mA | 120 |
| 83 | mG*mA*mUmAmUmAmGmGfAfAfUmUmAmAmGmAmGmGmA*mU*mA | 212 |
| | mU*fA*mUmCfCmUfCmUmUmAmAmUmUfCmCfUmAmUmAmUmC*mA*mC | 122 |
| 84 | mA*mC*mAmGmAmUmUmGfCfUfGmCmUmUmCmUmGmCmU*mA*mA | 213 |
| | mU*fU*mAmGfCmAfGmAmAmGmCmAmGfCmAfAmUmCmUmGmU*mA*mC | 124 |
| 107 | mA*mG*mCmAmAmAmAmCfCfAfUmUmGmCmUmUmCmAmC*mU*mA | 172 |
| | mU*fA*mGmUfGmAfAmGmCmAmAmUmGfGmUfUmUmUmGmCmU*mG | 215 |
| 108 | mA*mG*mCmAmAmAmAmCfCfAfUmUmGmCmUmUmCmAmC*mU*mA | 172 |
| | mU*fA*mGmUfGmAfAmGmCmAmAmUmGfGmUfUmUmUmGmC*mU*mG*mU | 216 |
| 109 | mA*mG*mCmAmAmAmAmCfCfAfUmUmGmCmUmUmCmAmC*mU*mA | 172 |
| | mU*fA*mGmUfGmAfAmGmCmAmAmUmGfGmUfUmUmUmGmCmU*mG*mU[iAb] | 217 |
| 110 | mA*mG*mCmAmAmAmAmCfCfAfUmUmGmCmUmUmCmAmC*mU*mA | 172 |
| | mU*fA*mGmUfGmAfAmGmCmAmAmUmGfGmUfUmUmUmGmC*mU*mG | 218 |

Abbreviations-“m” indicates 2′-OMe; “f” indicates 2′-fluoro; “*” indicates phosphorothioate linkage; “VP″ indicates 5′-vinylphosphonate; “iAb” indicates inverted abasic moiety in Table 6; “n” indicates an abasic moiety; “d” indicates a 2′-deoxy; “S” means the sense strand; “AS” means the antisense strand; unless otherwise noted, the 5′ position of the AS can include 5′-phosphate or 5′-vinylphosphonate.

TABLE 7b
Modified Nucleic Acid Sequences of dsRNA targeting the coding sequence of
human APP mRNA

| | | SEQ |
|---|---|---|
| dsRNA | | ID |
| No. | Oligo Sequence 5′ to 3′ | NO |
| 85 | mC*mC*mAmAmGmAfUmGfCfAfGmCmAmGmAmAmCmGmG*mC*mA | 125 |
| | mU*fG*mCmCmGfUmUmCmUmGmCmUmGfCmAfUmCmUmUmGmG*mA*mC | 126 |
| 86 | mA*mG*mGmAmAmGfCmAfGfCfCmAmAmCmGmAmGmAmG*mA*mA | 127 |
| | mU*fU*mCmUmCfUmCmGmUmUmGmGmCfUmGfCmUmUmCmCmU*mG*mU | 128 |
| 87 | mC*mU*mUmUmGmAfGmCfAfGfAmUmGmCmAmGmAmAmC*mU*mA | 129 |
| | mU*fA*mGmUmUfCmUmGmCmAmUmCmUfGmCfUmCmAmAmAmG*mA*mA | 130 |
| 88 | mG*mG*mCmAmGmUfUmAfUfCfCmAmGmCmAmUmUmUmC*mC*mA | 131 |
| | mU*fG*mGmAmAfAmUmGmCmUmGmGmAfUmAfAmCmUmGmCmC*mU*mU | 132 |
| 89 | mU*mC*mCmAmAmCfCmUfAfCfAmAmGmUmUmCmUmUmU*mG*mA | 133 |
| | mU*fC*mAmAmAfGmAmAmCmUmUmGmUfAmGfGmUmUmGmGmA*mU*mU | 134 |
| 90 | mC*mC*mAmAmCmCfUmAfCfAfAmGmUmUmCmUmUmUmG*mA*mA | 135 |
| | mU*fU*mCmAmAfAmGmAmAmCmUmUmGfUmAfGmGmUmUmGmG*mA*mU | 136 |
| 91 | mC*mU*mGmAmAmGfAmAfGfAfAmAmCmAmGmUmAmCmA*mC*mA | 137 |
| | mU*fG*mUmGmUfAmCmUmGmUmUmUmCfUmUfCmUmUmCmAmG*mC*mA | 138 |
| 92 | mG*mA*mCmAmAmAfGmUfAfGfUmAmGmAmAmGmUmAmG*mC*mA | 139 |
| | mU*fG*mCmUmAfCmUmUmCmUmAmCmUfAmCfUmUmUmGmUmC*mU*mU | 140 |
| 93 | mA*mA*mGmUmUmCfUmUfUfGfAmGmCmAmGmAmUmGmC*mA*mA | 141 |
| | mU*fU*mGmCmAfUmCmUmGmCmUmCmAfAmAfGmAmAmCmUmU*mG*mU | 142 |
| 94 | mA*mG*mGmCmAmGfUmUfAfUfCmCmAmGmCmAmUmUmU*mC*mA | 143 |
| | mU*fG*mAmAmAfUmGmCmUmGmGmAmUfAmAfCmUmGmCmCmU*mU*mC | 144 |
| 95 | mA*mG*mCmAmCmCfGmAfGfAfGmAmGmAmAmUmGmUmC*mC*mA | 145 |
| | mU*fG*mGmAmCfAmUmUmCmUmCmUmCfUmCfGmGmUmGmCmU*mU*mG | 146 |
| 96 | mC*mC*mGmGmUmCfCmCfAfGfGmUmUmAmUmGmAmCmA*mC*mA | 147 |
| | mU*fG*mUmGmUfCmAmUmAmAmCmCmUfGmGfGmAmCmCmGmG*mA*mU | 148 |
| 97 | mA*mA*mCmCmAmGfUmGfAfCfCmAmUmCmCmAmGmAmA*mC*mA | 149 |
| | mU*fG*mUmUmCfUmGmGmAmUmGmGmUfCmAfCmUmGmGmUmU*mG*mG | 150 |
| 98 | mA*mC*mCmGmAmGfGmAfCfUfGmAmCmCmAmCmUmCmG*mA*mA | 151 |
| | mU*fU*mCmGmAfGmUmGmGmUmCmAmGfUmCfCmUmCmGmGmU*mC*mG | 152 |
| 99 | mC*mA*mCmAmCmCfGmUfCfGfCmCmAmAmAmGmAmGmA*mC*mA | 153 |
| | mU*fG*mUmCmUfCmUmUmUmGmGmCmGfAmCfGmGmUmGmUmG*mC*mC | 154 |
| 100 | mG*mC*mCmAmAmGfCmAfCfCfGmAmGmAmGmAmGmAmA*mU*mA | 155 |
| | mU*fA*mUmUmCfUmCmUmCmUmCmGmGfUmGfCmUmUmGmGmC*mC*mU | 156 |
| 101 | mG*mA*mUmCmCmGfGmUfCfCfCmAmGmGmUmUmAmUmG*mA*mA | 157 |
| | mU*fU*mCmAmUfAmAmCmCmUmGmGmGfAmCfCmGmGmAmUmC*mU*mG | 158 |
| 102 | mA*mA*mGmCmAmCfCmGfAfGfAmGmAmGmAmAmUmGmU*mC*mA | 159 |
| | mU*fG*mAmCmAfUmUmCmUmCmUmCmUfCmGfGmUmGmCmUmU*mG*mG | 160 |
| 103 | mC*mC*mAmUmCmCfAmGfAfAfCmUmGmGmUmGmCmAmA*mG*mA | 161 |
| | mU*fC*mUmUmGfCmAmCmCmAmGmUmUfCmUfGmGmAmUmGmG*mU*mC | 162 |
| 104 | mG*mU*mCmCmAmAfGmAfUfGfCmAmGmCmAmGmAmAmC*mG*mA | 163 |
| | mU*fC*mGmUmUfCmUmGmCmUmGmCmAfUmCfUmUmGmGmAmC*mA*mG | 164 |
| 105 | mC*mA*mGmGmUmCfAmUfGfAfGmAmGmAmAmUmGmGmG*mA*mA | 165 |
| | mU*fU*mCmCmCfAmUmUmCmUmCmUmCfAmUfGmAmCmCmUmG*mG*mG | 166 |
| 111 | mC*mC*mAmAmGmAmUmGfCfAfGmCmAmGmAmAmCmGmG*mC*mA | 219 |
| | mU*fG*mCmCfGmUfUmCmUmGmCmUmGfCmAfUmCmUmUmGmG*mA*mC | 220 |
| 112 | mA*mG*mGmAmAmGmCmAfGfCfCmAmAmCmGmAmGmAmG*mA*mA | 221 |
| | mU*fU*mCmUfCmUfCmGmUmUmGmGmCfUmGfCmUmUmCmCmU*mG*mU | 222 |
| 113 | mC*mU*mUmUmGmAmGmCfAfGfAmUmGmCmAmGmAmAmC*mU*mA | 223 |
| | mU*fA*mGmUfUmCfUmGmCmAmUmCmUfGmCfUmCmAmAmAmG*mA*mA | 224 |
| 114 | mG*mG*mCmAmGmUmUmAfUfCfCmAmGmCmAmUmUmUmC*mC*mA | 225 |
| | mU*fG*mGmAfAmAfUmGmCmUmGmGmAfUmAfAmCmUmGmCmC*mU*mU | 226 |
| 115 | mG*mA*mCmAmAmAmGmUfAfGfUmAmGmAmAmGmUmAmG*mC*mA | 227 |
| | mU*fG*mCmUfAmCfUmUmCmUmAmCmUfAmCfUmUmUmGmUmC*mU*mU | 228 |
| 116 | mA*mG*mGmCmAmGmUmUfAfUfCmCmAmGmCmAmUmUmU*mC*mA | 229 |
| | mU*fG*mAmAfAmUfGmCmUmGmGmAmUfAmAfCmUmGmCmCmU*mU*mC | 230 |
| 117 | mA*mG*mCmAmCmCmGmAfGfAfGmAmGmAmAmUmGmUmC*mC*mA | 231 |
| | mU*fG*mGmAfCmAfUmUmCmUmCmUmCfUmCfGmGmUmGmCmU*mU*mG | 232 |
| 118 | mA*mA*mCmCmAmGmUmGfAfCfCmAmUmCmCmAmGmAmA*mC*mA | 233 |
| | mU*fG*mUmUfCmUfGmGmAmUmGmGmUfCmAfCmUmGmGmUmU*mG*mG | 234 |
| 119 | mA*mC*mCmGmAmGmGmAfCfUfGmAmCmCmAmCmUmCmG*mA*mA | 235 |
| | mU*fU*mCmGfAmGfUmGmGmUmCmAmGfUmCfCmUmCmGmGmU*mC*mG | 236 |
| 120 | mC*mA*mCmAmCmCmGmUfCfGfCmCmAmAmAmGmAmGmA*mC*mA | 237 |
| | mU*fG*mUmCfUmCfUmUmUmGmGmCmGfAmCfGmGmUmGmUmG*mC*mC | 238 |
| 121 | mG*mC*mCmAmAmGmCmAfCfCfGmAmGmAmGmAmGmAmA*mU*mA | 239 |
| | mU*fA*mUmUfCmUfCmUmCmUmCmGmGfUmGfCmUmUmGmGmC*mC*mU | 240 |
| 122 | mG*mA*mUmCmCmGmGmUfCfCfCmAmGmGmUmUmAmUmG*mA*mA | 241 |
| | mU*fU*mCmAfUmAfAmCmCmUmGmGmGfAmCfCmGmGmAmUmC*mU*mG | 242 |

Abbreviations-“m” indicates 2′-OMe; “f” indicates 2′-fluoro; “*” indicates phosphorothioate linkage; “VP″ indicates 5′-vinylphosphonate; “iAb” indicates inverted abasic moiety in Table 6; “n” indicates an abasic moiety; “d” indicates a 2′-deoxy; “S” means the sense strand; “AS” means the antisense strand; unless otherwise noted, the 5′ position of the AS can include 5′-phosphate or 5′-vinylphosphonate.

In some embodiments, the sense strand and the antisense strand of the dsRNA comprise a pair of nucleic acid sequences selected from the group consisting of:
(a) the sense strand comprises SEQ ID NO: 101, and the antisense strand comprises SEQ ID NO: 102, 173, 176, 177, 178, 179, 180, 182, or 185;
(b) the sense strand comprises SEQ ID NO: 103, and the antisense strand comprises SEQ ID NO: 104, 175, 189, 190, 191, 192, 194, 195, or 196;
(c) the sense strand comprises SEQ ID NO: 105, and the antisense strand comprises SEQ ID NO: 106;
(d) the sense strand comprises SEQ ID NO: 107, and the antisense strand comprises SEQ ID NO: 108;
(e) the sense strand comprises SEQ ID NO: 109, and the antisense strand comprises SEQ ID NO: 110;
(f) the sense strand comprises SEQ ID NO: 111, and the antisense strand comprises SEQ ID NO: 112;
(g) the sense strand comprises SEQ ID NO: 113, and the antisense strand comprises SEQ ID NO: 114;
(h) the sense strand comprises SEQ ID NO: 115, and the antisense strand comprises SEQ ID NO: 116;
(i) the sense strand comprises SEQ ID NO: 117, and the antisense strand comprises SEQ ID NO: 118;
(j) the sense strand comprises SEQ ID NO: 119, and the antisense strand comprises SEQ ID NO: 120;
(k) the sense strand comprises SEQ ID NO: 121, and the antisense strand comprises SEQ ID NO: 122;
(l) the sense strand comprises SEQ ID NO: 123, and the antisense strand comprises SEQ ID NO: 124;
(m) the sense strand comprises SEQ ID NO: 125, and the antisense strand comprises SEQ ID NO: 126;
(n) the sense strand comprises SEQ ID NO: 127, and the antisense strand comprises SEQ ID NO: 128;
(o) the sense strand comprises SEQ ID NO: 129, and the antisense strand comprises SEQ ID NO: 130;
(p) the sense strand comprises SEQ ID NO: 131, and the antisense strand comprises SEQ ID NO: 132;
(q) the sense strand comprises SEQ ID NO: 133, and the antisense strand comprises SEQ ID NO: 134;
(r) the sense strand comprises SEQ ID NO: 135, and the antisense strand comprises SEQ ID NO: 136;
(s) the sense strand comprises SEQ ID NO: 137, and the antisense strand comprises SEQ ID NO: 138;
(t) the sense strand comprises SEQ ID NO: 139, and the antisense strand comprises SEQ ID NO: 140;
(u) the sense strand comprises SEQ ID NO: 141, and the antisense strand comprises SEQ ID NO: 142;
(v) the sense strand comprises SEQ ID NO: 143, and the antisense strand comprises SEQ ID NO: 144;
(w) the sense strand comprises SEQ ID NO: 145, and the antisense strand comprises SEQ ID NO: 146;
(x) the sense strand comprises SEQ ID NO: 147, and the antisense strand comprises SEQ ID NO: 148;
(y) the sense strand comprises SEQ ID NO: 149, and the antisense strand comprises SEQ ID NO: 150;
(z) the sense strand comprises SEQ ID NO: 151, and the antisense strand comprises SEQ ID NO: 152;
(aa) the sense strand comprises SEQ ID NO: 153, and the antisense strand comprises SEQ ID NO: 154;
(bb) the sense strand comprises SEQ ID NO: 155, and the antisense strand comprises SEQ ID NO: 156;
(cc) the sense strand comprises SEQ ID NO: 157, and the antisense strand comprises SEQ ID NO: 158;
(dd) the sense strand comprises SEQ ID NO: 159, and the antisense strand comprises SEQ ID NO: 160;
(ee) the sense strand comprises SEQ ID NO: 161, and the antisense strand comprises SEQ ID NO: 162;
(ff) the sense strand comprises SEQ ID NO: 163, and the antisense strand comprises SEQ ID NO: 164;
(gg) the sense strand comprises SEQ ID NO: 165, and the antisense strand comprises SEQ ID NO: 166;
(hh) the sense strand comprises SEQ ID NO: 172, and the antisense strand comprises SEQ ID NO: 173;
(ii) the sense strand comprises SEQ ID NO: 181, and the antisense strand comprises SEQ ID NO: 173;
(jj) the sense strand comprises SEQ ID NO: 174 or 193, and the antisense strand comprises SEQ ID NO: 175;
(kk) the sense strand comprises SEQ ID NO: 172, and the antisense strand comprises SEQ ID NO: 177;
(ll) the sense strand comprises SEQ ID NO: 181, 183, or 186, and the antisense strand comprises SEQ ID NO: 102;
(mm) the sense strand comprises SEQ ID NO: 187, 193, or 197, and the antisense strand comprises SEQ ID NO: 104;
(nn) the sense strand comprises SEQ ID NO: 198, and the antisense strand comprises SEQ ID NO: 199;
(oo) the sense strand comprises SEQ ID NO: 200, and the antisense strand comprises SEQ ID NO: 201;
(pp) the sense strand comprises SEQ ID NO: 202, and the antisense strand comprises SEQ ID NO: 203;
(qq) the sense strand comprises SEQ ID NO: 204, and the antisense strand comprises SEQ ID NO: 205;
(rr) the sense strand comprises SEQ ID NO: 206, and the antisense strand comprises SEQ ID NO: 207;
(ss) the sense strand comprises SEQ ID NO: 208, and the antisense strand comprises SEQ ID NO: 209;
(tt) the sense strand comprises SEQ ID NO: 210, and the antisense strand comprises SEQ ID NO: 118;
(uu) the sense strand comprises SEQ ID NO: 211, and the antisense strand comprises SEQ ID NO: 120;
(vv) the sense strand comprises SEQ ID NO: 212, and the antisense strand comprises SEQ ID NO: 122;
(ww) the sense strand comprises SEQ ID NO: 213, and the antisense strand comprises SEQ ID NO: 124;
(xx) the sense strand comprises SEQ ID NO: 172, and the antisense strand comprises SEQ ID NO: 215;
(yy) the sense strand comprises SEQ ID NO: 172, and the antisense strand comprises SEQ ID NO: 216;
(zz) the sense strand comprises SEQ ID NO: 172, and the antisense strand comprises SEQ ID NO: 217;
(aaa) the sense strand comprises SEQ ID NO: 172, and the antisense strand comprises SEQ ID NO: 218;
(bbb) the sense strand comprises SEQ ID NO: 219, and the antisense strand comprises SEQ ID NO: 220;
(ccc) the sense strand comprises SEQ ID NO: 221, and the antisense strand comprises SEQ ID NO: 222;
(ddd) the sense strand comprises SEQ ID NO: 223, and the antisense strand comprises SEQ ID NO: 224;
(eee) the sense strand comprises SEQ ID NO: 225, and the antisense strand comprises SEQ ID NO: 226;
(fff) the sense strand comprises SEQ ID NO: 227, and the antisense strand comprises SEQ ID NO: 228;
(ggg) the sense strand comprises SEQ ID NO: 229, and the antisense strand comprises SEQ ID NO: 230;
(hhh) the sense strand comprises SEQ ID NO: 231, and the antisense strand comprises SEQ ID NO: 232;
(iii) the sense strand comprises SEQ ID NO: 233, and the antisense strand comprises SEQ ID NO: 234;
(jjj) the sense strand comprises SEQ ID NO: 235, and the antisense strand comprises SEQ ID NO: 236;
(kkk) the sense strand comprises SEQ ID NO: 237, and the antisense strand comprises SEQ ID NO: 238;
(lll) the sense strand comprises SEQ ID NO: 239, and the antisense strand comprises SEQ ID NO: 240; and
(mmm) the sense strand comprises SEQ ID NO: 241, and the antisense strand comprises SEQ ID NO: 242.

In some embodiments, the sense strand and the antisense strand of the dsRNA have a pair of nucleic acid sequences selected from the group consisting of:
(a) the sense strand consists of SEQ ID NO: 101, and the antisense strand consists of SEQ ID NO: 102, 173, 176, 177, 178, 179, 180, 182, or 185;
(b) the sense strand consists of SEQ ID NO: 103, and the antisense strand consists of SEQ ID NO: 104, 175, 189, 190, 191, 192, 194, 195, or 196;
(c) the sense strand consists of SEQ ID NO: 105, and the antisense strand consists of SEQ ID NO: 106;
(d) the sense strand consists of SEQ ID NO: 107, and the antisense strand consists of SEQ ID NO: 108;
(e) the sense strand consists of SEQ ID NO: 109, and the antisense strand consists of SEQ ID NO: 110;
(f) the sense strand consists of SEQ ID NO: 111, and the antisense strand consists of SEQ ID NO: 112;
(g) the sense strand consists of SEQ ID NO: 113, and the antisense strand consists of SEQ ID NO: 114;
(h) the sense strand consists of SEQ ID NO: 115, and the antisense strand consists of SEQ ID NO: 116;
(i) the sense strand consists of SEQ ID NO: 117, and the antisense strand consists of SEQ ID NO: 118;
(j) the sense strand consists of SEQ ID NO: 119, and the antisense strand consists of SEQ ID NO: 120;
(k) the sense strand consists of SEQ ID NO: 121, and the antisense strand consists of SEQ ID NO: 122;
(l) the sense strand consists of SEQ ID NO: 123, and the antisense strand consists of SEQ ID NO: 124;
(m) the sense strand consists of SEQ ID NO: 125, and the antisense strand consists of SEQ ID NO: 126;
(n) the sense strand consists of SEQ ID NO: 127, and the antisense strand consists of SEQ ID NO: 128;
(o) the sense strand consists of SEQ ID NO: 129, and the antisense strand consists of SEQ ID NO: 130;
(p) the sense strand consists of SEQ ID NO: 131, and the antisense strand consists of SEQ ID NO: 132;
(q) the sense strand consists of SEQ ID NO: 133, and the antisense strand consists of SEQ ID NO: 134;
(r) the sense strand consists of SEQ ID NO: 135, and the antisense strand consists of SEQ ID NO: 136;
(s) the sense strand consists of SEQ ID NO: 137, and the antisense strand consists of SEQ ID NO: 138;
(t) the sense strand consists of SEQ ID NO: 139, and the antisense strand consists of SEQ ID NO: 140;
(u) the sense strand consists of SEQ ID NO: 141, and the antisense strand consists of SEQ ID NO: 142;
(v) the sense strand consists of SEQ ID NO: 143, and the antisense strand consists of SEQ ID NO: 144;
(w) the sense strand consists of SEQ ID NO: 145, and the antisense strand consists of SEQ ID NO: 146;
(x) the sense strand consists of SEQ ID NO: 147, and the antisense strand consists of SEQ ID NO: 148;
(y) the sense strand consists of SEQ ID NO: 149, and the antisense strand consists of SEQ ID NO: 150;
(z) the sense strand consists of SEQ ID NO: 151, and the antisense strand consists of SEQ ID NO: 152;
(aa) the sense strand consists of SEQ ID NO: 153, and the antisense strand consists of SEQ ID NO: 154;
(bb) the sense strand consists of SEQ ID NO: 155, and the antisense strand consists of SEQ ID NO: 156;
(cc) the sense strand consists of SEQ ID NO: 157, and the antisense strand consists of SEQ ID NO: 158;
(dd) the sense strand consists of SEQ ID NO: 159, and the antisense strand consists of SEQ ID NO: 160;
(ee) the sense strand consists of SEQ ID NO: 161, and the antisense strand consists of SEQ ID NO: 162;
(ff) the sense strand consists of SEQ ID NO: 163, and the antisense strand consists of SEQ ID NO: 164;
(gg) the sense strand consists of SEQ ID NO: 165, and the antisense strand consists of SEQ ID NO: 166, (hh) the sense strand consists of SEQ ID NO: 172, and the antisense strand consists of SEQ ID NO: 173;
(ii) the sense strand consists of SEQ ID NO: 181, and the antisense strand consists of SEQ ID NO: 173;
(jj) the sense strand consists of SEQ ID NO: 174 or 193, and the antisense strand consists of SEQ ID NO: 175;
(kk) the sense strand consists of SEQ ID NO: 172, and the antisense strand consists of SEQ ID NO: 177;
(ll) the sense strand consists of SEQ ID NO: 181, 183, or 186, and the antisense strand consists of SEQ ID NO: 102;
(mm) the sense strand consists of SEQ ID NO: 187, 193, or 197, and the antisense strand consists of SEQ ID NO: 104;
(nn) the sense strand consists of SEQ ID NO: 198, and the antisense strand consists of SEQ ID NO: 199;
(oo) the sense strand consists of SEQ ID NO: 200, and the antisense strand consists of SEQ ID NO: 201;
(pp) the sense strand consists of SEQ ID NO: 202, and the antisense strand consists of SEQ ID NO: 203;
(qq) the sense strand consists of SEQ ID NO: 204, and the antisense strand consists of SEQ ID NO: 205;
(rr) the sense strand consists of SEQ ID NO: 206, and the antisense strand consists of SEQ ID NO: 207;
(ss) the sense strand consists of SEQ ID NO: 208, and the antisense strand consists of SEQ ID NO: 209;
(tt) the sense strand consists of SEQ ID NO: 210, and the antisense strand consists of SEQ ID NO: 118;
(uu) the sense strand consists of SEQ ID NO: 211, and the antisense strand consists of SEQ ID NO: 120;
(vv) the sense strand consists of SEQ ID NO: 212, and the antisense strand consists of SEQ ID NO: 122;
(ww) the sense strand consists of SEQ ID NO: 213, and the antisense strand consists of SEQ ID NO: 124;
(xx) the sense strand consists of SEQ ID NO: 172, and the antisense strand consists of SEQ ID NO: 215;
(yy) the sense strand consists of SEQ ID NO: 172, and the antisense strand consists of SEQ ID NO: 216;
(zz) the sense strand consists of SEQ ID NO: 172, and the antisense strand consists of SEQ ID NO: 217;
(aaa) the sense strand consists of SEQ ID NO: 172, and the antisense strand consists of SEQ ID NO: 218;
(bbb) the sense strand consists of SEQ ID NO: 219, and the antisense strand consists of SEQ ID NO: 220;
(ccc) the sense strand consists of SEQ ID NO: 221, and the antisense strand consists of SEQ ID NO: 222;
(ddd) the sense strand consists of SEQ ID NO: 223, and the antisense strand consists of SEQ ID NO: 224;
(eee) the sense strand consists of SEQ ID NO: 225, and the antisense strand consists of SEQ ID NO: 226;
(fff) the sense strand consists of SEQ ID NO: 227, and the antisense strand consists of SEQ ID NO: 228;
(ggg) the sense strand consists of SEQ ID NO: 229, and the antisense strand consists of SEQ ID NO: 230;
(hhh) the sense strand consists of SEQ ID NO: 231, and the antisense strand consists of SEQ ID NO: 232;
(iii) the sense strand consists of SEQ ID NO: 233, and the antisense strand consists of SEQ ID NO: 234;
(jjj) the sense strand consists of SEQ ID NO: 235, and the antisense strand consists of SEQ ID NO: 236;
(kkk) the sense strand consists of SEQ ID NO: 237, and the antisense strand consists of SEQ ID NO: 238;
(lll) the sense strand consists of SEQ ID NO: 239, and the antisense strand consists of SEQ ID NO: 240; and
(mmm) the sense strand consists of SEQ ID NO: 241, and the antisense strand consists of SEQ ID NO: 242.

In some embodiments, the sense strand comprises SEQ ID NO: 172, and the antisense strand comprises SEQ ID NO: 217. In some embodiments, the sense strand consists of SEQ ID NO: 172, and the antisense strand consists of SEQ ID NO: 217.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

The RNAi agent described herein can be made by a variety of procedures known to one of ordinary skill in the art, some of which are illustrated in the preparations and examples below, e.g., in Examples 1-3. One of ordinary skill in the art recognizes that the specific synthetic steps for each of the routes described may be combined in different ways, or in conjunction with steps from different schemes, to prepare the RNAi agent. The product of each step can be recovered by conventional methods well known in the art, including extraction, evaporation, precipitation, chromatography, filtration, trituration, and crystallization. The reagents and starting materials are readily available to one of ordinary skill in the art.

In some embodiments, the TfR binding protein with native or engineered cysteines described herein can be first treated with a reducing agent, e.g., DTT, and then re-oxidized with an oxidizing agent, e.g., DHAA. The resulting oxidized TfR binding protein is then incubated with a linker functionalized dsRNA, e.g., linker-dsRNA, to produce the conjugated RNAi agent.

### Pharmaceutical Composition

In another aspect, provided herein are pharmaceutical compositions comprising any of the APP RNAi agents described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating an APP associated neurologic disease in a patient in need thereof, and such the method comprises administering to the patient an effective amount of the APP RNAi agent or a pharmaceutical composition described herein. In some embodiments, the APP associated neurological disease is selected from Alzheimer's disease, Down's syndrome, or cerebral amyloid angiopathy. The APP RNAi agent or a pharmaceutical composition comprising APP RNAi agent can be administered to the patient intravenously or subcutaneously.

APP RNAi agent dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are APP RNAi agents or pharmaceutical compositions comprising an APP RNAi agent for use in a therapy. Also provided herein are APP RNAi agents or pharmaceutical compositions comprising an APP RNAi agent for use in the treatment of an APP associated neurological disease, e.g., Alzheimer's disease, Down's syndrome, or cerebral amyloid angiopathy. Also provided herein are uses of the APP RNAi agent in the manufacture of a medicament for treating an APP associated neurological disease, e.g., Alzheimer's disease, Down's syndrome, or cerebral amyloid angiopathy.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “antigen binding domain”, as used herein, refers to a portion of an antibody or antibody fragment that binds an antigen or an epitope of the antigen. For example. “TfR binding domain” refers to a portion of an antibody or antibody fragment that binds TfR or an epitope of TfR.

The term “heterodimeric antibody”, as used herein, refers to an antibody that comprises two distinct antigen-binding domains.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

As used herein, “APP” (also known as amyloid beta precursor protein or ABPP) refers to an amyloid precursor protein (APP) mRNA transcript, protein, or polypeptide. The nucleotide sequences of human APP mRNA transcript variants and amino acid sequences of human APP protein isoforms can be found at:
a. NM_000484.4 transcript variant 1→NP_000475.1 APP protein isoform a (the longest isoform);
b. NM_201413.3 transcript variant 2→NP_958816.1 APP protein isoform b;
c. NM_201414.3 transcript variant 3→NP_958817.1 APP protein isoform c;
d. NM_001136016.3 transcript variant 4→NP_001129488.1 APP protein isoform d;
e. NM_001136129.3 transcript variant 5→NP_001129601.1 APP protein isoform e;
f. NM_001136130.3 transcript variant 6→NP_001129602.1 APP protein isoform f,
g. NM_001136131.3 transcript variant 7→NP_001129603.1 APP protein isoform g;
h. NM_001204301.2 transcript variant 8→NP_001191230.1 APP protein isoform h;
i. NM_001204302.2 transcript variant 9→NP_001191231.1 APP protein isoform i;
j. NM_001204303.2 transcript variant 10→NP_001191232.1 APP protein isoform j;
k. NM_001385253.1 transcript variant 11→NP_001372182.1 APP protein isoform k.
   The amino acid sequence of human APP protein isoform a (longest isoform) can be found at NP 000475.1:

| (SEQ ID NO: 167) | |
|---|---|
| 1 | MLPGLALLLLAAWTARALEVPTDGNAGLLAEPQIAMFCGRLNMHMNVQNGKWDSDPSGTK |
| 61 | TCIDTKEGILQYCQEVYPELQITNVVEANQPVTIQNWCKRGRKQCKTHPHFVIPYRCLVG |
| 121 | EFVSDALLVPDKCKFLHQERMDVCETHLHWHTVAKETCSEKSTNLHDYGMLLPCGIDKFR |
| 181 | GVEFVCCPLAEESDNVDSADAEEDDSDVWWGGADTDYADGSEDKVVEVAEEEEVAEVEEE |
| 241 | EADDDEDDEDGDEVEEEAEEPYEEATERTTSIATTTTTTTESVEEVVREVCSEQAETGPC |
| 301 | RAMISRWYFDVTEGKCAPFFYGGCGGNRNNFDTEEYCMAVCGSAMSQSLLKTTQEPLARD |
| 361 | PVKLPTTAASTPDAVDKYLETPGDENEHAHFQKAKERLEAKHRERMSQVMREWEEAERQA |
| 421 | KNLPKADKKAVIQHFQEKVESLEQEAANERQQLVETHMARVEAMLNDRRRLALENYITAL |
| 481 | QAVPPRPRHVFNMLKKYVRAEQKDRQHTLKHFEHVRMVDPKKAAQIRSQVMTHLRVIYER |
| 541 | MNQSLSLLYNVPAVAEEIQDEVDELLQKEQNYSDDVLANMISEPRISYGNDALMPSLTET |
| 601 | KTTVELLPVNGEFSLDDLQPWHSFGADSVPANTENEVEPVDARPAADRGLTTRPGSGLTN |
| 661 | IKTEEISEVKMDAEFRHDSGYEVHHQKLVFFAEDVGSNKGAIIGLMVGGVVIATVIVITL |
| 721 | VMLKKKQYTSIHHGVVEVDAAVTPEERHLSKMQQNGYENPTYKFFEQMQN. |

The human APP mRNA transcript variant 1 sequence encoding human APP protein isoform a (longest isoform) can be found at NM_000484.4:

| (SEQ ID NO: 168) | |
|---|---|
| 1 | GTCAGTTTCC TCGGCAGCGG TAGGCGAGAG CACGCGGAGG AGCGTGCGCG GGGGCCCCGG |
| 61 | GAGACGGCGG CGGTGGCGGC GCGGGCAGAG CAAGGACGCG GCGGATCCCA CTCGCACAGC |
| 121 | AGCGCACTCG GTGCCCCGCG CAGGGTCGCG ATGCTGCCCG GTTTGGCACT GCTCCTGCTG |
| 181 | GCCGCCTGGA CGGCTCGGGC GCTGGAGGTA CCCACTGATG GTAATGCTGG CCTGCTGGCT |
| 241 | GAACCCCAGA TTGCCATGTT CTGTGGCAGA CTGAACATGC ACATGAATGT CCAGAATGGG |
| 301 | AAGTGGGATT CAGATCCATC AGGGACCAAA ACCTGCATTG ATACCAAGGA AGGCATCCTG |
| 361 | CAGTATTGCC AAGAAGTCTA CCCTGAACTG CAGATCACCA ATGTGGTAGA AGCCAACCAA |
| 421 | CCAGTGACCA TCCAGAACTG GTGCAAGCGG GGCCGCAAGC AGTGCAAGAC CCATCCCCAC |
| 481 | TTTGTGATTC CCTACCGCTG CTTAGTTGGT GAGTTTGTAA GTGATGCCCT TCTCGTTCCT |
| 541 | GACAAGTGCA AATTCTTACA CCAGGAGAGG ATGGATGTTT GCGAAACTCA TCTTCACTGG |
| 601 | CACACCGTCG CCAAAGAGAC ATGCAGTGAG AAGAGTACCA ACTTGCATGA CTACGGCATG |
| 661 | TTGCTGCCCT GCGGAATTGA CAAGTTCCGA GGGGTAGAGT TTGTGTGTTG CCCACTGGCT |
| 721 | GAAGAAAGTG ACAATGTGGA TTCTGCTGAT GCGGAGGAGG ATGACTCGGA TGTCTGGTGG |
| 781 | GGCGGAGCAG ACACAGACTA TGCAGATGGG AGTGAAGACA AAGTAGTAGA AGTAGCAGAG |
| 841 | GAGGAAGAAG TGGCTGAGGT GGAAGAAGAA GAAGCCGATG ATGACGAGGA CGATGAGGAT |
| 901 | GGTGATGAGG TAGAGGAAGA GGCTGAGGAA CCCTACGAAG AAGCCACAGA GAGAACCACC |
| 961 | AGCATTGCCA CCACCACCAC CACCACCACA GAGTCTGTGG AAGAGGTGGT TCGAGAGGTG |
| 1021 | TGCTCTGAAC AAGCCGAGAC GGGGCCGTGC CGAGCAATGA TCTCCCGCTG GTACTTTGAT |
| 1081 | GTGACTGAAG GGAAGTGTGC CCCATTCTTT TACGGCGGAT GTGGCGGCAA CCGGAACAAC |
| 1141 | TTTGACACAG AAGAGTACTG CATGGCCGTG TGTGGCAGCG CCATGTCCCA AAGTTTACTC |
| 1201 | AAGACTACCC AGGAACCTCT TGCCCGAGAT CCTGTTAAAC TTCCTACAAC AGCAGCCAGT |
| 1261 | ACCCCTGATG CCGTTGACAA GTATCTCGAG ACACCTGGGG ATGAGAATGA ACATGCCCAT |
| 1321 | TTCCAGAAAG CCAAAGAGAG GCTTGAGGCC AAGCACCGAG AGAGAATGTC CCAGGTCATG |
| 1381 | AGAGAATGGG AAGAGGCAGA ACGTCAAGCA AAGAACTTGC CTAAAGCTGA TAAGAAGGCA |
| 1441 | GTTATCCAGC ATTTCCAGGA GAAAGTGGAA TCTTTGGAAC AGGAAGCAGC CAACGAGAGA |
| 1501 | CAGCAGCTGG TGGAGACACA CATGGCCAGA GTGGAAGCCA TGCTCAATGA CCGCCGCCGC |
| 1561 | CTGGCCCTGG AGAACTACAT CACCGCTCTG CAGGCTGTTC CTCCTCGGCC TCGTCACGTG |
| 1621 | TTCAATATGC TAAAGAAGTA TGTCCGCGCA GAACAGAAGG ACAGACAGCA CACCCTAAAG |
| 1681 | CATTTCGAGC ATGTGCGCAT GGTGGATCCC AAGAAAGCCG CTCAGATCCG GTCCCAGGTT |
| 1741 | ATGACACACC TCCGTGTGAT TTATGAGCGC ATGAATCAGT CTCTCTCCCT GCTCTACAAC |
| 1801 | GTGCCTGCAG TGGCCGAGGA GATTCAGGAT GAAGTTGATG AGCTGCTTCA GAAAGAGCAA |
| 1861 | AACTATTCAG ATGACGTCTT GGCCAACATG ATTAGTGAAC CAAGGATCAG TTACGGAAAC |
| 1921 | GATGCTCTCA TGCCATCTTT GACCGAAACG AAAACCACCG TGGAGCTCCT TCCCGTGAAT |
| 1981 | GGAGAGTTCA GCCTGGACGA TCTCCAGCCG TGGCATTCTT TTGGGGCTGA CTCTGTGCCA |
| 2041 | GCCAACACAG AAAACGAAGT TGAGCCTGTT GATGCCCGCC CTGCTGCCGA CCGAGGACTG |
| 2101 | ACCACTCGAC CAGGTTCTGG GTTGACAAAT ATCAAGACGG AGGAGATCTC TGAAGTGAAG |
| 2161 | ATGGATGCAG AATTCCGACA TGACTCAGGA TATGAAGTTC ATCATCAAAA ATTGGTGTTC |
| 2221 | TTTGCAGAAG ATGTGGGTTC AAACAAAGGT GCAATCATTG GACTCATGGT GGGCGGTGTT |
| 2281 | GTCATAGCGA CAGTGATCGT CATCACCTTG GTGATGCTGA AGAAGAAACA GTACACATCC |
| 2341 | ATTCATCATG GTGTGGTGGA GGTTGACGCC GCTGTCACCC CAGAGGAGCG CCACCTGTCC |
| 2401 | AAGATGCAGC AGAACGGCTA CGAAAATCCA ACCTACAAGT TCTTTGAGCA GATGCAGAAC |
| 2461 | TAGACCCCCG CCACAGCAGC CTCTGAAGTT GGACAGCAAA ACCATTGCTT CACTACCCAT |
| 2521 | CGGTGTCCAT TTATAGAATA ATGTGGGAAG AAACAAACCC GTTTTATGAT TTACTCATTA |
| 2581 | TCGCCTTTTG ACAGCTGTGC TGTAACACAA GTAGATGCCT GAACTTGAAT TAATCCACAC |
| 2641 | ATCAGTAATG TATTCTATCT CTCTTTACAT TTTGGTCTCT ATACTACATT ATTAATGGGT |
| 2701 | TTTGTGTACT GTAAAGAATT TAGCTGTATC AAACTAGTGC ATGAATAGAT TCTCTCCTGA |
| 2761 | TTATTTATCA CATAGCCCCT TAGCCAGTTG TATATTATTC TTGTGGTTTG TGACCCAATT |
| 2821 | AAGTCCTACT TTACATATGC TTTAAGAATC GATGGGGGAT GCTTCATGTG AACGTGGGAG |
| 2881 | TTCAGCTGCT TCTCTTGCCT AAGTATTCCT TTCCTGATCA CTATGCATTT TAAAGTTAAA |
| 2941 | CATTTTTAAG TATTTCAGAT GCTTTAGAGA GATTTTTTTT CCATGACTGC ATTTTACTGT |
| 3001 | ACAGATTGCT GCTTCTGCTA TATTTGTGAT ATAGGAATTA AGAGGATACA CACGTTTGTT |
| 3061 | TCTTCGTGCC TGTTTTATGT GCACACATTA GGCATTGAGA CTTCAAGCTT TTCTTTTTTT |
| 3121 | GTCCACGTAT CTTTGGGTCT TTGATAAAGA AAAGAATCCC TGTTCATTGT AAGCACTTTT |
| 3181 | ACGGGGGGGG TGGGGAGGGG TGCTCTGCTG GTCTTCAATT ACCAAGAATT CTCCAAAACA |
| 3241 | ATTTTCTGCA GGATGATTGT ACAGAATCAT TGCTTATGAC ATGATCGCTT TCTACACTGT |
| 3301 | ATTACATAAA TAAATTAAAT AAAATAACCC CGGGCAAGAC TTTTCTTTGA AGGATGACTA |
| 3361 | CAGACATTAA ATAATCGAAG TAATTTTGGG TGGGGAGAAG AGGCAGATTC AATTTTCTTT |
| 3421 | AACCAGTCTG AAGTTTCATT TATGATACAA AAGAAGATGA AAATGGAAGT GGCAATATAA |
| 3481 | GGGGATGAGG AAGGCATGCC TGGACAAACC CTTCTTTTAA GATGTGTCTT CAATTTGTAT |
| 3541 | AAAATGGTGT TTTCATGTAA ATAAATACAT TCTTGGAGGA GCA. |

The nucleic acid sequence of a mouse APP mRNA transcript can be found at NM_001198823.1; and the amino acid sequence of a mouse APP protein can be found at NP_001185752.1. The nucleic acid sequence of a rat APP mRNA transcript can be found at NM_019288.2; and the amino acid sequence of a rat APP protein can be found at NP_062161.1. The nucleic acid sequence of a monkey APP mRNA transcript can be found at XM_015133068.2; and the amino acid sequence of a monkey APP protein can be found at XP_014988554.1.

As used herein, the term “APP associated neurological disease” refers to a neurological disease characterized by extracellular amyloid deposits or plaques.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene or target after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, 2′ deoxy nucleotide (DNA), or 2′-O-alkyl modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 6.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, a “null arm” means an antibody arm that does not bind any known human target.

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ end of an oligonucleotide in place of a 5′-phosphate, which is sometimes susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “polypeptide” or “protein”, as used herein, refers to a polymer of amino acid residues. The term applies to polymers comprising naturally occurring amino acids and polymers comprising one or more non-naturally occurring amino acids.

As used herein, “RNAi,” “RNAi agent,” “iRNA,” “iRNA agent,” or “RNA interference agent” means an agent that mediates sequence-specific degradation of a target mRNA by RNA interference, e.g., via RNA-induced silencing complex (RISC) pathway. In some embodiments, the RNAi agent has a sense strand and an antisense strand, and the sense strand and the antisense strand form a duplex (e.g., a double stranded RNA).

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The following examples are offered to illustrate, but not to limit, the claimed inventions.

### EXAMPLES

### Example 1: Generation and Characterization of TfR Binding Proteins

### Generation of Human TfR Binding Proteins

Antibody against human TfR was generated by immunizing AlivaMab® transgenic mice with the extracellular domains of human Transferrin Receptor 1 protein with a His tag (hR-ECD-6His, SEQ TD NO: 170, see Table 8) and mouse Transferrin Receptor protein (mTfR, SEQ ID NO: 169). Antigen positive B-cells were sorted from pooled spleens. Binding of individual antibodies cloned from those B-cells to his-tagged hTfR-ECD was verified.

Additional antibody against human TfR was generated by immunizing AlivaMab® transgenic mice with the apical domain of human Transferrin Receptor 1 protein with a His tag (hTfR-ApD-6His, SEQ TD NO: 171, see Table 8). Antigen positive B-cells were sorted from pooled spleens. Binding of individual antibodies cloned from those B-cells to his-tagged hTfR-ECD was verified.

TABLE 8
Sequences of the immunogens used to generate human or mouse TfR antibodies.

| Immunogen | Sequence | SEQ ID NO |
|---|---|---|
| mTfR-ECD-6His | HHHHHHCKRVEQKEECVKLAETEETDKSETMETEDV | 169 |
| | PTSSRLYWADLKTLLSEKLNSIEFADTIKQLSQNTYTP | |
| | REAGSQKDESLAYYIENQFHEFKFSKVWRDEHYVKI | |
| | QVKSSIGQNMVTIVQSNGNLDPVESPEGYVAFSKPTE | |
| | VSGKLVHANFGTKKDFEELSYSVNGSLVIVRAGEITF | |
| | AEKVANAQSFNAIGVLIYMDKNKFPVVEADLALFGH | |
| | AHLGTGDPYTPGFPSFNHTQFPPSQSSGLPNIPVQTISR | |
| | AAAEKLFGKMEGSCPARWNIDSSCKLELSQNQNVKL | |
| | IVKNVLKERRILNIFGVIKGYEEPDRYVVVGAQRDAL | |
| | GAGVAAKSSVGTGLLLKLAQVFSDMISKDGFRPSRSII | |
| | FASWTAGDFGAVGATEWLEGYLSSLHLKAFTYINLD | |
| | KVVLGTSNFKVSASPLLYTLMGKIMQDVKHPVDGKS | |
| | LYRDSNWISKVEKLSFDNAAYPFLAYSGIPAVSFCFCE | |
| | DADYPYLGTRLDTYEALTQKVPQLNQMVRTAAEVA | |
| | GQLIIKLTHDVELNLDYEMYNSKLLSFMKDLNQFKT | |
| | DIRDMGLSLOWLYSARGDYFRATSRLTTDFHNAEKT | |
| | NRFVMREINDRIMKVEYHFLSPYVSPRESPFRHIFWGS | |
| | GSHTLSALVENLKLRQKNITAFNETLFRNQLALATWT | |
| | IQGVANALSGDIWNIDNEF | |
| hTfR-ECD-6His | HHHHHHCKGVEPKTECERLAGTESPVREEPGEDFPA | 170 |
| | ARRLYWDDLKRKLSEKLDSTDFTGTIKLLNENSYVPR | |
| | EAGSQKDENLALYVENQFREFKLSKVWRDQHFVKIQ | |
| | VKDSAQNSVIIVDKNGRLVYLVENPGGYVAYSKAAT | |
| | VTGKLVHANFGTKKDFEDLYTPVNGSIVIVRAGKITF | |
| | AEKVANAESLNAIGVLIYMDQTKFPIVNAELSFFGHA | |
| | HLGTGDPYTPGFPSFNHTQFPPSRSSGLPNIPVQTISRA | |
| | AAEKLFGNMEGDCPSDWKTDSTCRMVTSESKNVKL | |
| | TVSNVLKEIKILNIFGVIKGFVEPDHYVVVGAQRDAW | |
| | GPGAAKSGVGTALLLKLAQMFSDMVLKDGFQPSRSII | |
| | FASWSAGDFGSVGATEWLEGYLSSLHLKAFTYINLD | |
| | KAVLGTSNFKVSASPLLYTLIEKTMQNVKHPVTGQFL | |
| | YQDSNWASKVEKLTLDNAAFPFLAYSGIPAVSFCFCE | |
| | DTDYPYLGTTMDTYKELIERIPELNKVARAAAEVAG | |
| | QFVIKLTHDVELNLDYERYNSQLLSFVRDLNQYRADI | |
| | KEMGLSLOWLYSARGDFFRATSRLTTDFGNAEKTDR | |
| | FVMKKLNDRVMRVEYHFLSPYVSPKESPFRHVFWGS | |
| | GSHTLPALLENLKLRKQNNGAFNETLFRNQLALATW | |
| | TIQGAANALSGDVWDIDNEF | |
| hTfR-ApD-6His | HHHHHHHHGKPIPNPLLGLDSTGGGGSDSAQNSVIIV | 171 |
| | DKNGRLVYLVENPGGYVAYSKAATVTGKLVHANFG | |
| | TKKDFEDLYTPVNGSIVIVRAGKITFAEKVANAESLN | |
| | AIGVLIYMDQTKFPIVNAELSFFGHAHLGGGGGGLPN | |
| | IPVQTISRAAAEKLFGNMEGDCPSDWKTDSTCRMVTS | |
| | ESKNVKLTVS | |

Affinity variants of the generated human TfR antibodies were made by systematically introducing mutations into individual CDR of each antibody and the resulting variants were subjected to multiple rounds of selection with decreasing concentrations of antigen and/or increasing periods of dissociation to isolate clones with improved affinities. The sequences of individual variants were used to construct a combinatorial library which was subjected to an additional round of selection with increased stringency to identify additive or synergistic mutational pairings between the individual CDR regions. Individual combinatorial clones are sequenced. The heavy chain and light chain CDRs and VH/VL sequences of the human TfR binding domains and proteins are provided in Table 1a.

Human TfR binding proteins were generated by recombinant DNA technology. Such human TfR binding proteins can be expressed in a mammalian cell line such as HEK293 or CHO, either transiently or stably transfected with an expression system using an optimal predetermined HC:LC vector ratio or a single vector system encoding both HC and LC. Clarified media, into which the protein has been secreted, can be purified using the commonly used techniques.

### Binding Affinity

Binding affinity and binding stoichiometry of the exemplified human TfR binding proteins to human and Cynomolgus TfR was characterized using a surface plasmon resonance assay on a Biacore 8K instrument primed with HBS-EP+ (10 mM Hepes pH7.4+150 mM NaCl+3 mM EDTA+0.05% (w/v) surfactant P20) running buffer and analysis temperature set at 37° C. Target human and Cynomolgous TfR ECDs were immobilized on a CM4 chip (Cytiva P/N 29104989) using standard NHS-EDC amine coupling. The TfR binding proteins were prepared at a final concentration of 0.3, 0.1, 0.033, 0.01, 0.0033, 0.001, 0.00033, 0.0001 μM respectively by dilution of stock solution into running buffer.

Binding analysis was performed in a multi-cycle kinetics manner. Each analysis cycle consists of (1) injection of the lowest to highest concentration proteins over all Fc at 50 μL/min for 140 seconds followed by return to buffer flow for 400 seconds to monitor dissociation phase; (2) regeneration of chip surfaces with injection of 3M magnesium chloride, for 30 seconds at 100 μL/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 9.

TABLE 9
Binding Affinity of Exemplified human TfR binding proteins to human or
cynomolgus TfR at 37° C.

| | | Standard error | | Standard error of |
|---|---|---|---|---|
| Human TfR | | of the mean, | | the mean, Cyno |
| binding | Human TfR K_{D} | Human TfR K_{D} | Cyno TfR K_{D} | TfR K_{D} |
| proteins | (Biacore, nM) | (Biacore, nM) | (Biacore, nM) | (Biacore, nM) |
| (TBP) | at 37° C. | n = 3 | at 37° C. | n = 3 |
| TBP3 | 32.087 | 11.795 | 66.565 | 11.695 |
| TBP4 | 153.642 | 7.949 | 300.180 | 2.565 |
| TBP5 | 0.522 | 0.284 | 502.210 | 8.129 |

### Example 2: Synthesis and Characterization of dsRNAs Targeting APP

Single strands (sense and antisense) of the dsRNA duplexes were typically synthesized on solid support via a MerMade™ 12 (LGC Biosearch Technologies) or a similar automated oligonucleotide synthesizer. The sequences of the sense and antisense strands were shown in Table 5a or 5b. The sense strands were synthesized using an appropriate CPG such as 3′-Cholesterol-TEG CNA CPG 500 (LGC Biosearch Technologies) or phthalamido amino C6 lcaa CPG 500 Å (Chemgenes) whereas the antisense strands used standard support (LGC Biosearch Technologies). The oligonucleotides were synthesized via phosphoramidite chemistry at an appropriate scale for in-vitro or in-vivo experimentation.

Standard reagents were used in the oligo synthesis (Table 11), where 0.1M xanthane hydride in pyridine was used as the sulfurization reagent and 20% DEA in ACN was used as an auxiliary wash post synthesis. All monomers (Table 12a) were made at 0.1M in ACN and contained a molecular sieves trap bag. The structure of linked cholesterol is shown in Table 12b.

The antisense strands were typically cleaved and deprotected (C/D) at 45° C. for 16-24 hours. The sense strands were typically cleaved and deprotected from the CPG using cold 50% (methylamine/ammonia hydroxide 28-30%) at ambient temperature for 2-3 hrs, whereas 3% DEA in ammonia hydroxide (28-30%, cold) was typically used for the antisense strands. C/D was determined complete by IP-RP LC/MS when the resulting mass data confirmed the identity of sequence. RNA hydroxy desilylation may be carried out using triethylamine trihydrofluoride in DMSO. Dependent on scale, the CPG was filtered via 0.45 um PVDF syringeless filter, 0.22 μm PVDF Steriflip® vacuum filtration or 0.22 μm PVDF Stericup® Quick release. The CPG was typically back washed/rinsed with either 30% EtOH/RNAse free water then filtered through the same filtering device and combined with the first filtrate. This was repeated twice. The material was then divided evenly into conical centrifuge tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using anion-exchange (AEX) or reverse-phase (RP) chromatography. For AEX, an ES Industry Source™ 15Q column with MPA: 20 mM NaH₂PO₄, 15% ACN, pH 7.4 and MPB: 20 mM NaH₂PO₄, 1M NaBr, 15% ACN, pH 7.4. For RP, an ES Industry Source™ 15RPC with MPA: 50 mM sodium acetate, 10% ACN and MPB: 80% ACN. Fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K WCO centrifugal spin tubes at 3500×g for 2 m. Cholesterol-linked oligonucleotides were annealed at this stage to give cholesterol conjugated dsRNA by mixing equimolar aliquots of sense and antisense strands at room temperature for 30 minutes. The final desalted oligonucleotides were analyzed for concentration (nano drop at A260), characterized by IP-RP LC/MS for mass purity (Table 10) and UPLC for UV-purity.

TABLE 10
Exemplary LC/MS data

| | | TegChol | TegChol | C6-Amino | C6-Amino |
|---|---|---|---|---|---|
| dsRNA | | MW Cal. | MW Obs. | MW Cal. | MW Obs. |
| No.* | Strand | (g/mol)† | (g/mol)† | (g/mol)‡ | (g/mol)‡ |
| 48 | S: SEQ ID NO 172 | 7702.67 | 7703.3 | 7141.93 | 7142.6 |
| | AS: SEQ ID NO 173 | 7769.04 | 7770.0 | N/A | N/A |
| 50 | S: SEQ ID NO 174 | 7701.69 | 7702.7 | 7140.95 | 7141.3 |
| | AS: SEQ ID NO 175 | 7745.01 | 7745.4 | N/A | N/A |
| 63 | S: SEQ ID NO 172 | 7702.67 | 7702.5 | 7141.93 | 7142.6 |
| | AS: SEQ ID NO 177 | 7769.04 | 7769.0 | N/A | N/A |
| 75 | S: SEQ ID NO 198 | 7783.70 | 7783.6 | 7222.97 | 7223.0 |
| | AS: SEQ ID NO 199 | 7656.99 | 7657.3 | N/A | N/A |
| 76 | S: SEQ ID NO 200 | 7781.73 | 7782.1 | 7221.00 | 7221.1 |
| | AS: SEQ ID NO 201 | 7688.00 | 7687.9 | N/A | N/A |
| 77 | S: SEQ ID NO 202 | 7657.58 | 7657.5 | 7096.84 | 7096.8 |
| | AS: SEQ ID NO 203 | 7806.16 | 7806.4 | N/A | N/A |
| 78 | S: SEQ ID NO 204 | 7774.69 | 7774.4 | 7213.96 | 7213.8 |
| | AS: SEQ ID NO 205 | 7734.08 | 7734.0 | N/A | N/A |
| 79 | S: SEQ ID NO 206 | 7696.61 | 7696.5 | 7135.88 | 7135.9 |
| | AS: SEQ ID NO 207 | 7758.11 | 7757.8 | N/A | N/A |
| 80 | S: SEQ ID NO 208 | 7760.66 | 7760.5 | 7199.93 | 7199.6 |
| | AS: SEQ ID NO 209 | 7703.07 | 7702.7 | N/A | N/A |
| 81 | S: SEQ ID NO 210 | 7705.57 | 7705.6 | 7144.84 | 7144.4 |
| | AS: SEQ ID NO 118 | 7803.20 | 7802.8 | N/A | N/A |
| 82 | S: SEQ ID NO 211 | 7705.57 | 7705.6 | 7144.84 | 7144.4 |
| | AS: SEQ ID NO 120 | 7803.20 | 7802.9 | N/A | N/A |
| 83 | S: SEQ ID NO 212 | 7910.82 | 7910.5 | 7350.08 | 7350.1 |
| | AS: SEQ ID NO 122 | 7513.87 | 7513.6 | N/A | N/A |
| 84 | S: SEQ ID NO 213 | 7712.61 | 7712.5 | 7151.88 | 7151.9 |
| | AS: SEQ ID NO 124 | 7757.12 | 7757.0 | N/A | N/A |
| 107 | S: SEQ ID NO 172 | 7702.67 | 7702.7 | 7141.93 | 7142.6 |
| | AS: SEQ ID NO 215 | 7432.78 | 7432.7 | N/A | N/A |
| 108 | S: SEQ ID NO 172 | 7702.67 | 7702.9 | 7141.93 | 7142.6 |
| | AS: SEQ ID NO 216 | 7785.1 | 7785.3 | N/A | N/A |
| 109 | S: SEQ ID NO 172 | 7702.67 | 7702.9 | 7141.93 | 7142.6 |
| | AS: SEQ ID NO 217 | 7949.13 | 7949.6 | N/A | N/A |
| 110 | S: SEQ ID NO 172 | 7702.67 | 7702.9 | 7141.93 | 7142.6 |
| | AS: SEQ ID NO 218 | 7448.84 | 7449.2 | N/A | N/A |
| 111 | S: SEQ ID NO 219 | 7858.83 | 7859.2 | 7298.09 | 7298.0 |
| | AS: SEQ ID NO 220 | 7655.95 | 7656.1 | N/A | N/A |
| 113 | S: SEQ ID NO 223 | 7798.72 | 7799.3 | 7237.98 | 7238.1 |
| | AS: SEQ ID NO 224 | 7695.04 | 7695.3 | N/A | N/A |
| 114 | S: SEQ ID NO 225 | 7711.63 | 7711.9 | 7150.89 | 7150.8 |
| | AS: SEQ ID NO 226 | 7751.07 | 7751.3 | N/A | N/A |
| 115 | S: SEQ ID NO 227 | 7908.85 | 7908.9 | 7348.11 | 7347.9 |
| | AS: SEQ ID NO 228 | 7523.82 | 7524.1 | N/A | N/A |
| 116 | S: SEQ ID NO 229 | 7735.65 | 7735.8 | 7174.92 | 7174.9 |
| | AS: SEQ ID NO 230 | 7711.04 | 7711.3 | N/A | N/A |
| 120 | S: SEQ ID NO 237 | 7778.78 | 7779.1 | 7218.04 | 7218.1 |
| | AS: SEQ ID NO 238 | 7711.98 | 7712.2 | N/A | N/A |

*These dsRNAs have a 5′-(E)-vinylphosphonate on the antisense strand (AS).
†Sense strands (S) in this column are conjugated at the 3′-position to cholesterol entry 1 as shown in Table 12b.
‡Sense strands (S) in this column are conjugated at the 3′-position to a C6-amino moiety via phosphorothioate linkage. Antisense strand masses are listed as N/A in these columns as duplexation with the corresponding antisense strand takes place at a later step as in Example 3.

TABLE 11
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water, 70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 12a
Phosphoramidites

| Phosphoramidite | Abbreviation | Supplier | Catalog # | CAS |
|---|---|---|---|---|
| DMT-2′-F-A(Bz)-CE | fA | Hongene | PD1-001 | 136834-22-5 |
| Phosphoamidite | | | | |
| DMT-2′-F-C(Ac)-CE | fC | Hongene | PD3-001 | 159414-99-0 |
| Phosphoamidite | | | | |
| DMT-2′-F-G(iBu)-CE | fG | Hongene | PD2-002 | 144089-97-4 |
| Phosphoamidite | | | | |
| DMT-2′-F-U-CE | fU | Hongene | PD5-001 | 146954-75-8 |
| Phosphoamidite | | | | |
| DMT-2′-O-Me-A(Bz)- | mA | Hongene | PR1-001 | 110782-31-5 |
| CE Phosphoamidite | | | | |
| DMT-2′-O-Me-C(Ac)- | mC | Hongene | PR3-001 | 199593-09-4 |
| CE Phosphoamidite | | | | |
| DMT-2′-O-Me-G(iBu)- | mG | Hongene | PR2-002 | 150780-67-9 |
| CE Phosphoamidite | | | | |
| DMT-2′-O-Me-U-CE | mU | Hongene | PR5-001 | 110764-79-9 |
| Phosphoamidite | | | | |
| DMT-2′O-TBDMS- | A | Hongene | PR1-008 | 104992-55-4 |
| rA(bz) | | | | |
| Phosphoramidite | | | | |
| DMT-dC(Ac) | dC | Chemgenes | ANP-5560 | 154110-40-4 |
| Phosphoramidite | | | | |
| 5′bis(POM) vinyl | POM-VPmU | Hongene | PR5-032 | BVPMUP23B2A1 |
| phosphate-2′-Ome- | | | | |
| U3′CE | | | | |
| phosphoroamidite | | | | |
| Reverse Abasic | iAb | Chemgenes | ANP-1422 | 401813-16-9 |
| phosphoroamidite | | | | |
| Abasic | Aba | Chemgenes | ANP-7058 | 129821-76-7 |
| phosphoroamidite | | | | |

TABLE 12b
Linked Cholesterol Structures

| | Structure |
|---|---|
| 1 | 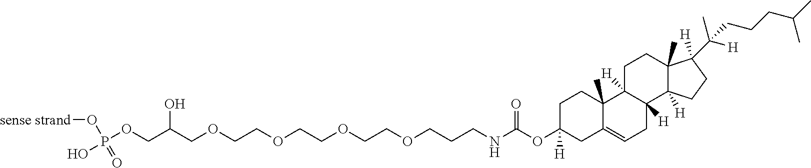 |
| 2 | 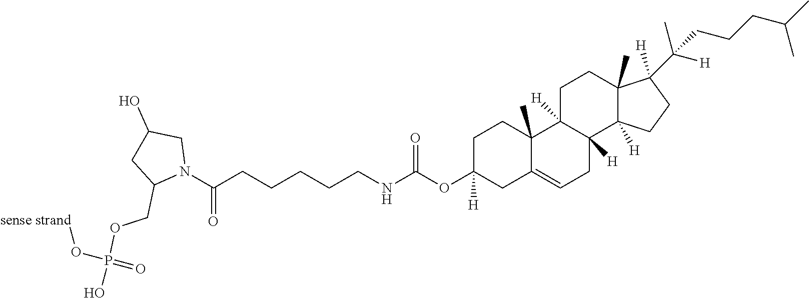 |

### Example 3: Generation of APP RNAi Agents

Certain abbreviations are defined as follows: “ACN” refers to acetonitrile; “aAEX” refers to analytical anion exchange; “APP” refers to amyloid precursor protein; “AS” refers to antisense strand; “CPG” refers to controlled pore glass; “DAR” refers to drug/siRNA to antibody/protein ratio; “DCM” refers to dichloromethane; “DEA” refers to diethylamine; “DHAA” refers to dehydroascorbic acid; “DMSO” refers to dimethylsulfoxide; “DMT” refers to dimethoxytrityl; “dsRNA” refers to double stranded ribonucleic acid; “DTT” refers to dithiothreitol; “EtOH” refers to ethanol; “h” refers to hours; “HPLC” refers to high-performance liquid chromatography; “IP-RP LCMS” refers to ion-pair reversed phase liquid chromatography mass spectrometry; “LC/MS” refers to liquid chromatography mass spectrometry; “LTQ/MS” refers to linear ion trap mass spectrometer; “min” refers to minutes; “MW” refers to molecular weight; “MWCO” refers to molecular weight cut-off, “NHS” refers to N-hydroxysuccinimide; “OD” refers to optical density; “PBS” phosphate-buffered saline; “PEG” refers to polyethylene glycol; “PVDF” refers to polyvinylidene fluoride; “RNAi” refers to RNA interference; “rpm” refers to revolutions per minute; “RT-qPCR” refers to reverse transcription-quantitative polymerase chain reaction; “SEC” refers to size exclusion chromatography; “siRNA” refers to small interfering RNA; “SMCC” refers to succinimidyl-4-(N-maleimidomethyl)cyclohexane-1-carboxylate; “SS” refers to sense strand; “TCO” refers to trans-cyclo-octene; “TfR” refers to transferrin receptor; “THF” refers to tetrahydrofuran; “TRIS” refers to tris(hydroxymethyl)aminomethane; “UPLC” refers to ultra performance liquid chromatography; and “UV” refers to ultraviolet.

SMCC-Functionalization of dsRNA

To a 50 mL conical tube containing amino-functionalized sense strand oligonucleotide SS-APP-AMINO, for example SEQ ID 172 appended to a C6-amino chain via a 3′-terminal phosphorothiolate ester, as a solution in water (8.42 mL, 0.023 mmol, 19.857 mg/mL), was added sodium bicarbonate powder (59 mg, 0.702 mmol). The mixture was briefly vortexed and sonicated to dissolve the bicarbonate. A freshly prepared solution of (2,5-dioxopyrrolidin-1-yl) 4-[(2,5-dioxopyrrol-1-yl)methyl]cyclohexanecarboxylate (96 mg, 0.281 mmol) in acetonitrile (6.32 mL) was then added to the bicarbonate-oligo solution, for example, dsRNA-48-PS-C6-amino (8.42 mL, 0.023 mmol, 19.857 mg/mL in water) and vortexed for 30 seconds. Then, the reaction was allowed to proceed for 4 hours with shaking at ambient temperature at 300 rpm, at which point temperature control on a ThermoMixer® C took the reaction mixture down to 10° C. for 15 hours. At this point, LTQ-MS analysis indicated full conversion. The reaction was quenched to pH 5 using 1N HC1 (621 μL, 0.621 mmol). The quenched reaction mixture was then concentrated to approximately ½ volume using a GeneVac™ centrifugal evaporator and the resultant precipitate-containing suspension was filtered using a 0.22 micron Steri-Flip® apparatus to remove precipitate, rinsing once with 5 mL of nuclease-free water. The resulting clear solution containing oligo was then diluted to approximately 55 mL with 20% acetonitrile in nuclease-free water and concentrated using a CentriCon® ultrafiltration apparatus (3000 MWCO regenerated cellulose membrane). Following passage of all the volume through the Centricon®, two more 55 mL portions of 20% acetonitrile in nuclease-free water were passed through the CentriCon® to rinse the material, and finally one passage of 55 mL pure Milli-Q® water to remove residual acetonitrile. The retentate was then recovered by inverting the Centricon® apparatus on the included recovery cup. The Centricon® apparatus was then washed and aspirated twice with 800 μL nuclease-free water in each of the two filtration pores (1.6 mL total per wash), and the combined rinsate and retentate were passed through a 50k MWCO filter, which was rinsed one more time with 5 mL nuclease-free water. Finally, the desired compound was measured for concentration using a NanoDrop™ apparatus (OD260—calculated extinction coefficient: 216.09 mmol-1 cm-1) to give the desired compound (SEQ ID 172 with appended C6-amino-SMCC) as a solution of 9.77 mg/mL in 13.219 mL (129 mg, 68.1%). LTQ-MS: observed deconvoluted m/z=7361.7, calculated mass 7361.17, mass purity 91.37%.

### SMCC-dsRNA Duplex

To a conical tube containing SS-APP-AMINO-SMCC, for example SEQ ID NO 172 with appended C6-Amino-SMCC (12.05 mL, 0.016 mmol, 1.328 mmol/L), was added its corresponding SS-APP-ANTISENSE, for example SEQ ID NO 173 with 5′-E-vinyl phosphonate, (0.0165 mmol, 2.619 mmol/L). The solutions were shaken at 25° C. for 30 minutes to give the desired SMCC-functionalized dsRNA (SMCC-dsRNA), then refrigerated to 10° C. for storage. The annealed solutions were sampled for LTQ purity and UPLC non-denaturing chromatography. Analysis via non-denaturing UPLC (run at 10° C.) shows a major single peak of 92% purity. LTQ-MS: (Antisense strand observed deconvoluted m/z=7768.4, calculated 7769.04; Sense strand observed deconvoluted m/z=7360.4, calculated mass 7361.17).

Conjugation Scheme for SMCC Functionalized dsRNA

The typical conjugation method utilized the SMCC-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This was followed by incubating the SMCC-dsRNA with the TfR binding proteins at 4 molar equivalents for overnight conjugation at 4° C.

Optionally, following conjugation a maleimide hydrolysis step can be done to secure the linker-payload in terminal stage and avoid deconjugation during human body circulation via retro-Michael addition. This succinimide ring hydrolysis process was done by elevating the conjugate pH to 9.0 using 50 mM Arginine (stock solution of 0.7M arginine, pH 9.0 was used) and incubating the solution at 37° C. for 20 hours. The hydrolysis state of the maleimide was confirmed by LCMS characterization of +18 Da that is incurred by the water addition to the succinimide ring.

Synthesis of Mal-Tet-TCO and GDM linkers and conjugation of Mal-Tet-TCO- or GDM linker-functionalized dsRNA to the engineered cysteine of the TfR binding proteins have been described in WO 2024/036096.

Conjugation was monitored using analytical anion exchange chromatography. A ProPac™ SAX-10 HPLC Column, 10 μm particle, 4 mm diameter, 250 mm length was utilized with the following method. Flow rate of 1 mL/min, Buffer A: 20 mM TRIS pH 7.0, Buffer B: 20 mM TRIS pH 7.0+1.5M NaCl, at 30° C.

Drug/siRNA to antibody/protein ratio (DAR) was calculated based on peak area % from the analytical anion exchange (aAEX) chromatogram.

Post conjugation of dsRNA to the TfR binding protein, excess dsRNA and unconjugated protein was removed by further purification. Either preparative size exclusion chromatography (SEC) or preparative anion exchange chromatography was utilized for purification of the final conjugate. Preparative SEC was performed using Cytiva Superdex® 200 in 1×PBS pH 7.2 under an isocratic condition. Alternatively, anion exchange, e.g., ThermoFisher POROS™ XQ, was used with starting buffer of 20 mM TRIS pH 7.0 and eluting with 20 column volume gradient with a buffer containing 20 mM TRIS pH 7.0 and 1M NaCl. These resulted in purified TfR binding protein-dsRNA conjugate devoid of excess dsRNA and minimal unconjugated protein. The resulting conjugate profile was analyzed by analytical anion exchange for final DAR quantitation (Table 13).

TABLE 13
siRNA/drug to TBP/antibody ratio (DAR)

| | Average | % of | % of | % of | % of |
|---|---|---|---|---|---|
| | DAR | DAR0 | DAR1 | DAR2 | DAR3 |
| TBP4-APP | 1.43 | 7.08% | 42.73% | 50.19% | N/A |
| siRNA | | | | | |
| conjugate | | | | | |
| (before | | | | | |
| purification) | | | | | |
| TBP4-APP | 1.79 | 2.21% | 36.55% | 47.67% | 15.69% |
| siRNA | | | | | |
| conjugate | | | | | |
| (after | | | | | |
| purification) | | | | | |
| TBP5-APP | 0.91 | 13.48% | 82.38% | 4.01% | N/A |
| siRNA | | | | | |
| conjugate | | | | | |
| (before | | | | | |
| purification) | | | | | |
| TBP5-APP | 1.0 | N/A | 100% | N/A | N/A |
| siRNA | | | | | |
| conjugate | | | | | |
| (after | | | | | |
| purification) | | | | | |

### Example 4: In Vitro Characterization of the APP RNAi Agents

In Vitro Potency Assessment of Cholesterol Conjugated dsRNTA Targeting APP in SHSY5Y and Mouse Cortical Neurons

Selected APP RNAi agents (cholesterol conjugated dsRNA targeting APP) were tested in vitro for APP inhibition in cultured SH-SY5Y cells and mouse cortical neurons.

### SH-SY5Y Cell Culture and RNAi Treatment and Analysis:

SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with additives including 10% fetal bovine serum. Cells were incubated at 37° C. in a humidified atmosphere of 5% CO₂. On day one, SH-SY5Y cells were plated in fibronectin coated tissue culture plates and allowed to attach overnight. On day two, complete media was removed and replaced with RNAi agent in serum free media. Cells were incubated with RNAi agent for 72 hours before analysis of gene (mRNA) expression. RT-qPCR was performed to quantify targeted mRNA levels using TaqMan™ Fast Advanced Cell-to-CT kit following the manufacturer's protocol (ThermoFisher A35377). The delta-delta CT method of normalizing to a housekeeping gene, GAPDH (ThermoFisher, Hs99999905_m1, GAPDH; Hs00169098_m1, APP), was used to determine relative amounts of gene (mRNA) expression. A three or four parameter logistic fit was used to determine IC₅₀.

### Mouse Primary Cortical Neuron (MCN) Culture and RNAi Treatment and Analysis:

Mouse primary cortical neurons were isolated from wild type C57BL6 mouse embryos at E18. On day 7, half of the medium was removed from each well and 2× concentration of RNAi agent in 2% FBS containing culture media with was added and incubated with cells for 7 days of treatment. At the end of treatment, RT-qPCR was performed to quantify targeted mRNA levels using TaqMan™ Fast Advanced Cell-to-CT kit. The delta-delta CT method of normalizing to a housekeeping gene, 3-actin probes (ThermoFisher, Mm02619580_g1, ACTB; Mm01344172_m1, APP), was used to determine relative amounts of gene (mRNA) expression. A three or four parameter logistic fit was used to determine IC₅₀.

As shown in Tables 14A, 14B and 15, cholesterol conjugated dsRNA targeting the APP coding region (Tables 14A and 14B) or 3′UTR (Table 15) successfully reduce human APP gene (mRNA) expression in SHSY5Y cells and mouse cortical neurons. Table 16 shows the efficacy of cholesterol conjugated dsRNA targeting APP with different 2′-fluoro modification patterns of either the sense strand or the antisense strand.

TABLE 14A
In vitro knock down (KD) of APP mRNA by cholesterol
conjugated dsRNA targeting APP coding sequence.

| | % APP mRNA KD at |
|---|---|
| | 1000 nM of RNAi agent in SH- |
| dsRNA No. | SY5Y cells (mean value, n = 2) |
| 95* | 59.8% |
| 94* | 80.9% |
| 92* | 77.6% |
| 97* | 60.6% |
| 98* | 57.8% |
| 99* | 64.4% |
| 100* | 66.8% |
| 101* | 55.5% |
| 88* | 88.1% |
| 87* | 77.3% |
| 86* | 54.8% |
| 85* | 75.2% |

*These dsRNAs have 5′ phosphate on the antisense strand and are conjugated to cholesterol entry 1 as shown in Table 12b.

TABLE 14B
In vitro IC50 of APP RNAi agent.

| | IC50 of APP RNAi agent in |
|---|---|
| dsRNA No. ‡ | SH-SY5Y cells (nM) |
| 117 ‡ | 17.6 |
| 116 ‡ | 3.79 |
| 115 ‡ | 6.65 |
| 118 ‡ | 31.7 |
| 119 ‡ | 96.9 |
| 120 ‡ | 9.36 |
| 121 ‡ | 33.8 |
| 122 ‡ | 36.8 |
| 114 ‡ | 3.68 |
| 113 ‡ | 7.95 |
| 112 ‡ | 20.1 |
| 111 ‡ | 3.48 |

‡ These dsRNAs have 5′-(E)-vinylphosphonate on the antisense strand and are conjugated to cholesterol entry 1 as shown in Table 12b.

TABLE 15
In vitro knock down of APP mRNA by cholesterol conjugated
dsRNA targeting APP 3′UTR

| | | | IC50 of |
|---|---|---|---|
| | % APP mRNA | | APP |
| | KD at 1000 nM of | IC50 of APP | RNAi |
| | RNAi agent in | RNAi agent | agent in |
| | SH-SY5Y cells | in SH-SY5Y | MCN |
| dsRNA No. | (mean value, n = 2) | cells (nM) | (nM) |
| 36† | 37.2% | 15.7 | 0.936 |
| 37† | 93.3% | 10.8 | 2.21 |
| 38† | 92.3% | 7.33 | 10.3 |
| 39† | 88.3% | 16.0 | 4.59 |
| 40† | 90.6% | 14.2 | 10.5 |
| 41† | 87.7% | 30.4 | 4.29 |
| 42† | 91.4% | 37.9 | 14.9 |
| 43† | 89.9% | 10.0 | 2.79 |
| 44‡ | ND* | 15.4 | ND |
| 45‡ | ND | 17.2 | ND |
| 46‡ | ND | 16.7 | ND |
| 47‡ | ND | 15.4 | ND |
| 48§ | ND | 0.625 | ND |
| 50§ | ND | 0.815 | ND |
| 75§ | ND | 0.322 | ND |
| 76§ | ND | 1.019 | ND |
| 77§ | ND | 0.683 | ND |
| 78§ | ND | 1.302 | ND |
| 79§ | ND | 1.71 | ND |
| 80§ | ND | 0.792 | ND |
| 81§ | ND | 1.016 | ND |
| 82§ | ND | 1.9 | ND |
| 83§ | ND | 2.732 | ND |
| 84§ | ND | 1.349 | ND |

*ND means not determined.
†These dsRNAs have 5′ phosphate on the antisense strand and are conjugated to cholesterol entry 1 as shown in Table 12b.
‡These dsRNAs have 5′ phosphate on the antisense strand and are conjugated to cholesterol entry 2 as shown in Table 12b.
§These dsRNAs have 5′-(E)-vinylphosphonate on the antisense strand and are conjugated to cholesterol entry 1 as shown in Table 12b.

TABLE 16
In vitro knock down of APP mRNA by cholesterol conjugated
dsRNA with different chemical modification patterns.

| | IC50 of APP | IC50 of |
|---|---|---|
| | RNAi agent | APP RNAi |
| dsRNA | in SH-SY5Y | agent in |
| No.† | cells (nM) | MCN (nM) |
| 52 | 8.22 | 5.04 |
| 53 | 13.0 | 8.15 |
| 54 | 11.1 | 6.43 |
| 55 | 23.9 | 7.28 |
| 56 | 10.2 | 3.51 |
| 57 | 12.0 | 3.6 |
| 58 | 7.29 | 2.1 |
| 60 | 12.8 | ND |
| 61 | 13.9 | ND |
| 62 | 11.1 | ND |
| 65 | 11.5 | 5.93 |
| 66 | 8.8 | 4.2 |
| 67 | 9.04 | 4.49 |
| 68 | 11.4 | 3.11 |
| 69 | 8.95 | 6.26 |
| 70 | 7.04 | 2.83 |
| 71 | 8.2 | ND* |
| 72 | 6.52 | ND |
| 73 | 11.7 | ND |
| 74 | 10.1 | ND |

In Vitro Potency Assessment of TfR binding protein-dsRNA conjugates targeting APP in EFO-21 and Mouse Cortical Neurons
*ND means not determined.
†These dsRNAs have 5′ phosphate on the antisense strand and are conjugated to cholesterol entry 1 as shown in Table 12b.

Selected APP RNAi agents (TfR binding protein-dsRNA conjugates targeting APP) were tested in vitro for APP inhibition in EFO-21 cells and mouse cortical neurons (MCN).

### EFO-21 Cell Culture and RNAi Treatment and Analysis:

EFO-21 cells (Simon, W. E., et al., 1983. J Natl Cancer Inst 70, 839-845) were derived from human ovarian carcinomas. The base medium was composed of RPMI supplemented with additives including 20% fetal bovine serum. Cells were incubated at 37° C. in a humidified atmosphere of 5% CO₂. On day one, EFO-21 cells were plated in tissue culture plates and allowed to attach overnight. On day two, media was removed and replaced with RNAi agent and 1.5% serum containing media. Cells were incubated with RNAi agent for 72 hours before analysis of gene (mRNA) expression. RT-qPCR was performed to quantify targeted mRNA levels using TaqMan™ Fast Advanced Cell-to-CT kit following the manufacturer's protocol (ThermoFisher A35377). The delta-delta CT method of normalizing to a housekeeping gene, GAPDH (ThermoFisher, Hs99999905_m1, GAPDH; Hs00169098_m1, APP), was used to determine relative amounts of gene (mRNA) expression. A three or four parameter logistic fit was used to determine IC₅₀.

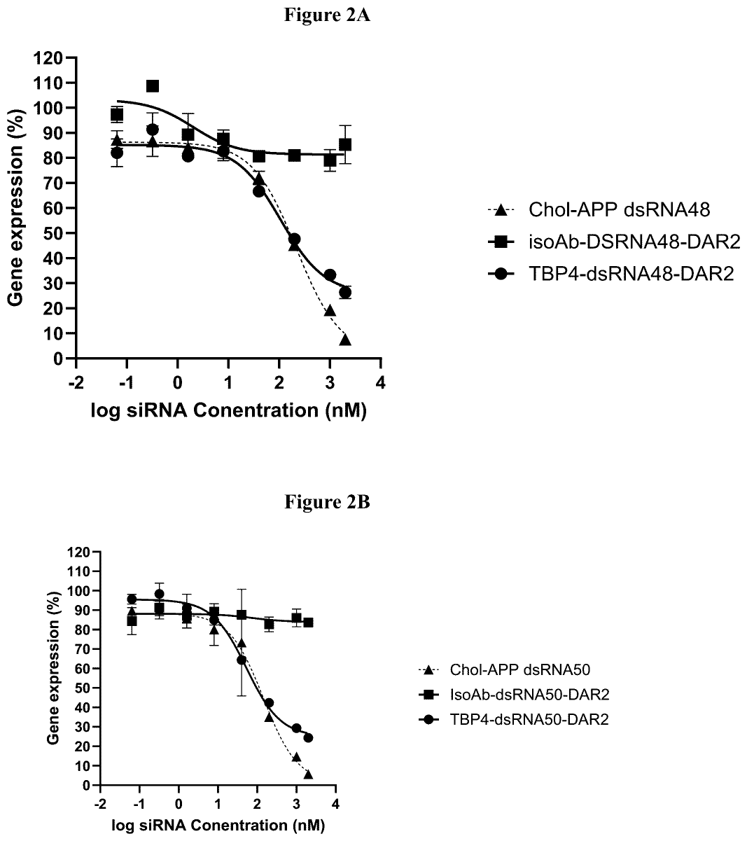

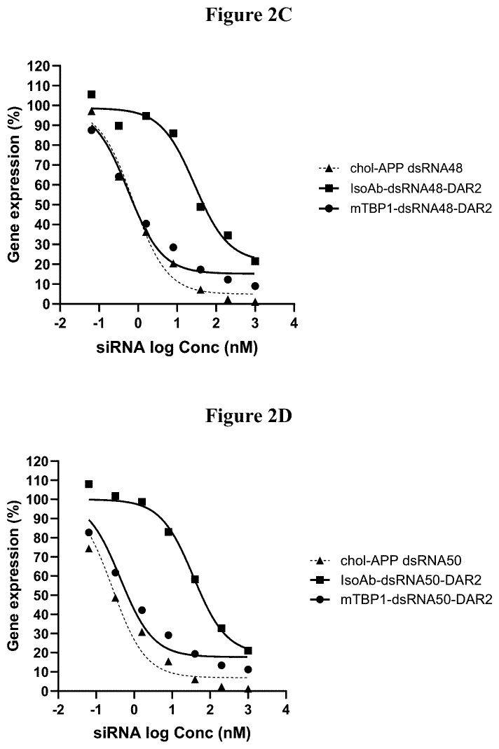

Results provided in FIGS. 2A-2B demonstrate that two human TfR binding protein-dsRNA conjugates successfully target human APP and reduces APP gene (mRNA) expression in EFO-21 cells. The potency of TfR binding protein-dsRNA conjugates is equivalent to the potency of cholesterol conjugated dsRNA. Binding to TfR via the TfR binding protein in the conjugates appears required for the observed gene silencing since an Isotype Ab-APP dsRNA did not show significant efficacy at any tested drug concentrations.

### Mouse Cortical Neurons and RNAi Treatment and Analysis.

Mouse primary cortical neurons were isolated from wild type C57BL6 mouse embryos at E18 and cultured as described above. On day 7, half of the medium was removed from each well and 2× concentration of dsRNA was added as either a cholesterol or antibody conjugated dsRNA (isotype antibody APP siRNA or mTBP1 antibody APP siRNA) in 2% FBS containing culture media was added and incubated with cells for 7 days of treatment. At the end of treatment, RT-qPCR was performed to quantify targeted mRNA levels using TaqMan™ Fast Advanced Cell-to-CT kit. The delta-delta CT method of normalizing to a housekeeping gene, 3-actin probes (ThermoFisher, Mm02619580_g1, ACTB; Mm01344172_m1, APP), was used to determine relative amounts of gene (mRNA) expression. A three or four parameter logistic fit was used to determine IC₅₀.

Results provided in FIGS. 2C and 2D demonstrate that two mouse TfR binding protein (mTBP1)-dsRNA conjugates successfully target mouse APP and reduces APP gene (mRNA) expression in primary mouse cortical neurons. The potency of mTBP1-dsRNA conjugates is similar to the potency of cholesterol conjugated dsRNA (FIGS. 2C-2D). The mTfR binding protein (mTBP1)-APP dsRNA conjugates show about 30-fold improvement in IC₅₀ over the Isotype Ab-APP dsRNA (FIGS. 2C and 2D). Overall, these results support that TfR-binding Ab enhance the potency of APP-siRNA in the therapeutically targeted-cell population, neuronal cells.

### Example 5: In Vivo Characterization of the APP RNAi Agents

In Vivo Potency Assessment of Cholesterol Conjugated dsRNA Targeting APP in Mouse after Single Intracerebroventricular (ICV) Dose

Selected APP RNAi agents (cholesterol conjugated dsRNA targeting APP) were also studied in wildtype C57BL/6N mice. Mice received ICV injection of 30 μg of the APP RNAi agent with different 2′-fluoro modification patterns (dsRNA No. 48 and dsRNA No. 63 in Table 7a) or PBS (phosphate buffered saline) and were sacrificed on Day 14 after the injection. Mouse APP mRNA expression in brain were measured and analyzed by qPCR, APP probe (Mm00431829_m1). The delta-delta CT method of normalizing used include housekeeping genes, 3-actin and GAPDH probes (Mm02619580_g1 and Mm99999915_g1, respectively). Protein expression was quantified using an immunoassay to assess Aβ(1-x), Aβ(1-40) and Aβ(1-42), peptide levels in homogenized brain tissues. Briefly, protein for Aβ peptide analysis was extracted from brain tissue using a Guanidine-HCL extraction protocol to capture both soluble and insoluble Aβ species. The assay used to detect Aβ(1-x) protein in brain homogenate was a standard sandwich enzyme-linked immunosorbent assay (ELISA) using commercially available or in-house generated antibodies and protein standards. Briefly, the capture antibody used was M266 (Haraln/Envigo) which recognizes Aβ(1-42) peptide aa 13-28 epitope. The detector antibody was an in-house generated biotinylated mouse specific Aβ(1-42) peptide aa 1-5 epitope antibody. The recombinant protein standard was rodent (rat) Aβ(1-42). ELISA assays were developed with UltraTMB-ELISA substrate (Thermo Scientific). Analyzed data was normalized to total protein concentration of brain sample and reported as pg/mg brain.

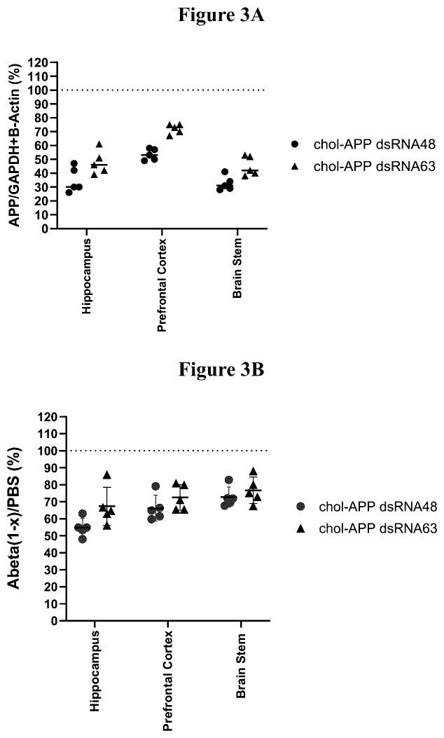

The results shown in FIGS. 3A and 3B exemplify efficacy of the tested APP RNAi agent 7 days after single ICV dose. FIG. 3A shows both APP RNAi agents reduce mouse APP gene (mRNA) expression (FIG. 3A) and protein expression levels (FIG. 3B) in AD relevant brain regions, such as hippocampus and prefrontal cortex. FIG. 3B demonstrates the reduction in the amyloid beta (Aβ) peptides (generated by secretase enzyme cleavage of APP protein) which aggregate and are the substrate of the extracellular amyloid plaques found in the brain tissue of people with Alzheimer's disease (AD), Down's syndrome, and cerebral amyloid angiopathy (CAA). Additionally, FIGS. 3A and 3B show the impact of different 2′-fluoro modification patterns of APP RNAi agent on APP gene silencing efficacy.

In Vivo Potency Assessment of TfR Binding Protein-dsRNA Conjugates Targeting APP in Mouse and Cynomolgus Monkey after Single Peripheral IV Dose.

To demonstrate that TfR binding protein-dsRNA conjugates cross the blood brain barrier (BBB) to deliver dsRNA cargo to the CNS, studies were conducted on select APP RNAi agents to assess pharmacodynamic efficacy and corresponding brain exposure after peripheral delivery via an intravenous route of delivery. Specifically, human TfR transgenic knock-in mice where the extracellular domain of transferrin-receptor have been humanized, received a single 10 mg/kg (dsRNA) IV dose of human TfR binding proteins-dsRNA targeting APP conjugates TBP4-dsRNA No. 48 (DAR2) or TBP5-sdRNA No. 48 (DAR1), or a PBS (phosphate buffered saline) control. Animals were sacrificed 28 days after injection. Brain samples were collected to assess pharmacodynamic efficacy and tissue exposure. To measure brain tissue exposure timepoints between 0.25 and 672 h (28 days) post-dose were collected and conjugate-associated dsRNA levels (ng/g) were quantified by reverse phase LC/MS after antibody-enrichment via immunoprecipitation (IP RP LC/MS).

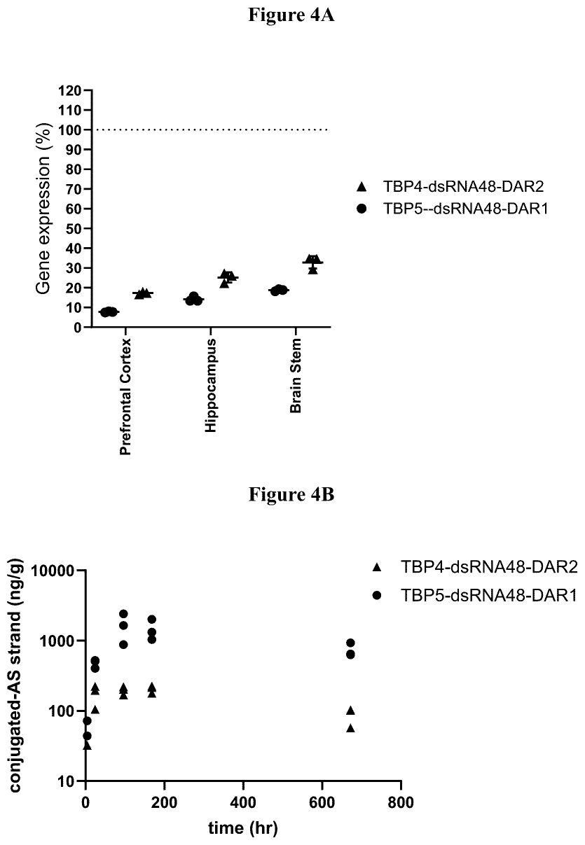

Results are shown in FIGS. 4A and 4B. A single IV administration of the APP RNAi agent results in reduced mouse APP mRNA levels in disease-relevant cortical and hippocampal regions (FIG. 4A). To understand the impact of DAR on the efficacy and brain exposure, TBP5-dsRNA No. 48 conjugate (DAR1) and TBP4-dsRNA No. 48 conjugate (DAR2) were dosed head-to-head in a comparator study. FIG. 4A shows that TBP5-dsRNA No. 48 conjugate (DAR1) reduced mouse APP mRNA levels by 92% and 85% in the prefrontal cortex and hippocampus regions, respectively. TBP4-dsRNA No. 48 conjugate-(DAR2) reduced mouse APP mRNA by 82% and 73% in the prefrontal cortex and hippocampus regions, respectively. Based on the AUC (0-672 h), FIG. 4B shows the DAR1 conjugate-associated dsRNA level in the brain tissue is 7.2-fold higher than the DAR2 conjugate associated dsRNA level.

To further demonstrate efficacy of TfR-shuttled conjugates, in vivo studies of dsRNA conjugates targeting different of APP CDS and 3′UTR sequences were also evaluated in the hTfR mouse. Table 17 reports reduction of APP mRNA levels (mean, n=4) in disease-relevant cortical and hippocampal regions 28 days after a single 1 mg/kg-IV bolus injection was achievable with multiple dsRNA targeting sequences.

TABLE 17
In vivo knock down of APP mRNA by TfR binding
protein-dsRNA conjugates
targeting APP CDS and 3′UTR sequences.

| | % mRNA | % mRNA |
|---|---|---|
| | Expression | Expression |
| TBP-dsRNA conjugates | (Cortex) | (Hippocampus) |
| TBP5-dsRNA77-DAR1 | 60 | 53 |
| TBP5-dsRNA78-DAR1 | 56 | 53 |
| TBP5-dsRNA84-DAR1 | 72 | 56 |
| TBP5-dsRNA111-DAR1 | 66 | 61 |
| TBP5-dsRNA114-DAR1 | 28 | 34 |
| TBP5-dsRNA116-DAR1 | 39 | 45 |
| TBP5-dsRNA120-DAR1 | 47 | 48 |

The efficacy of selected TfR binding protein-dsRNA conjugates were further tested in Cynomolgus monkey (Macaca fascicularis). To assess the efficacy Cynomolgus monkeys (four per group) received a single injection of 10 mg/kg (effective dsRNA concentration) in the Saphenous vein of the thigh. The monkeys were injected with either PBS (phosphate buffered saline) or the APP RNAi agent TBP4-dsRNA No. 48 (DAR2) or TBP5-dsRNA No. 48 (DAR1) and sacrificed 29 days after dosing. Deeply anesthetized animals underwent cardiac perfusion, then brain, spinal cord and peripheral tissues were collected. The perfused brain was coronally sectioned, and punches were collected from subregions including prefrontal cortex, temporal cortex, motor cortex, parietal cortex, hippocampus and frozen. Additional tissues collected from spinal cord, liver, kidney, and muscles were also collected. To assess target mRNA and protein levels by RT-qPCR and -ELISA respectively in tissue homogenates. mRNA expression levels of human APP were quantified via a delta-delta CT method with GAPDH being used as the housekeeping gene for CNS regions. Further, conjugate-associated dsRNA level in the brain tissue (28-day terminal) and plasma (various time-points between 0-672 h) were assessed by IP RP LC-MS. Protein for Aβ peptide analysis was extracted from brain tissue using a Guanidine-HCL extraction protocol to capture both soluble and insoluble Aβ species. The assay used to detect Aβ(1-x) protein in brain homogenate was a standard sandwich enzyme-linked immunosorbent assay (ELISA) using commercially available or in-house generated antibodies and protein standards. Briefly, the capture antibody used was M266 (Haraln/Envigo) which recognizes Aβ(1-42) peptide aa 13-28 epitope. The detector antibody for cyno was an in-house generated biotinylated 3D6 human/Cyno Aβ(1-42) peptide aa 1-5 epitope antibody. The recombinant protein standard used for Cyno was human Aβ(1-40). ELISA assays were developed with UltraTMB-ELISA substrate (Thermo Scientific). Analyzed data was normalized to total protein concentration of brain sample and reported as pg/mg brain.

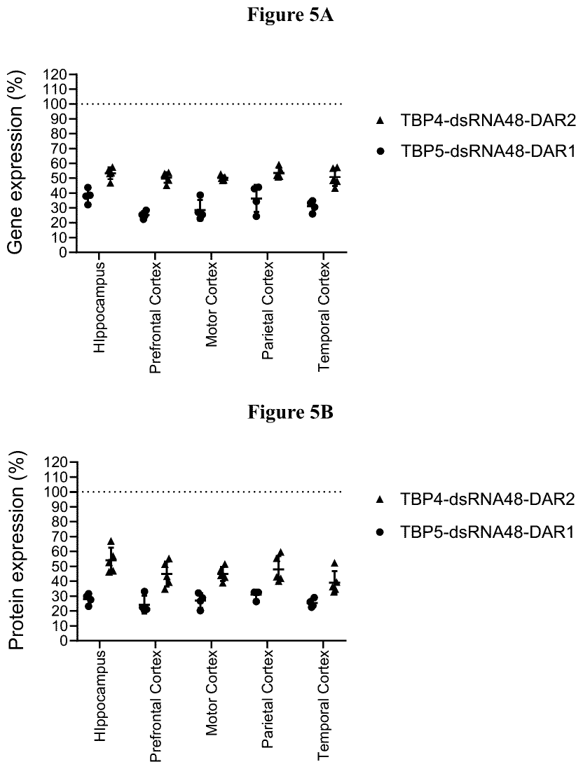

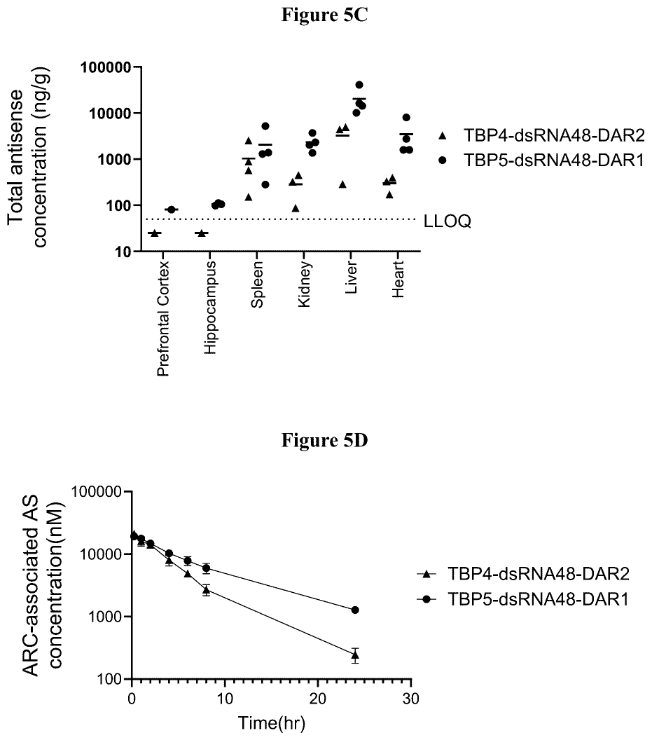

FIG. 5A shows APP mRNA reductions in disease relevant hippocampal and cortical regions 28 days after a single IV dose of TBP4-dsRNA No. 48 or TBP5-dsRNA No. 48. TBP5-dsRNA No. 48 treatment resulted in APP mRNA reductions of 62% in hippocampus, 75% in prefrontal cortex, 72% in motor cortex, 64% in parietal cortex, and 69% in temporal cortex. FIG. 5B shows the reduction of Aβ(1-x) protein, including Aβ(1-42) and Aβ(1-40), in key brain regions compared to the PBS treated control group 28 days post dose. The Aβ protein level reductions correlate to APP mRNA reductions in key tissues at 28 days, with 72% reduction in hippocampus, 76% reduction in prefrontal cortex, 73% reduction in motor cortex, 69% reduction in parietal cortex, and 75% reduction in temporal cortex. Similar reductions for other APP protein processing fragments, e.g., sAPPα were observed (data not shown). Assessment of peripheral tissues noted significant APP mRNA reductions in tissues such as gastrocnemius muscle and liver, but no significant reductions in kidney or spleen. Comparison of TBP4-dsRNA No. 48 (DAR2) and TBP5-dsRNA No. 48 (DAR1) showed higher efficacy of the TBP5-dsRNA No. 48 (DAR1) conjugate than the TBP4-dsRNA No. 48 (DAR2) conjugate (FIGS. 5A-5B), which is consistent with single dose mouse ICV study shown in FIG. 4A.

Assessment of the conjugate-associated dsRNA levels are presented in FIGS. 5C-D. The concentrations of the antisense strand of dsRNA at day 29 in the prefrontal cortical tissue and hippocampal tissues are 80.6 ng/g (n=1) and 104.9 ng/g (mean n=3) for TBP5-dsRNA No. 48 (DAR1) conjugate and were below detection threshold LLOQ for the TBP4-dsRNA No. 48 (DAR2) conjugate. These levels were 2-orders of magnitude lower than that observed in the spleen, kidney, and heart, and 3 orders of magnitude lower than that observed in the liver. The AUC (0-672 h) shows a 1.7-fold increase in plasma PK exposure for TBP5-dsRNA No. 48 (DAR1) conjugate than TBP4-dsRNA No. 48 (DAR2) conjugate at the same dsRNA dose (FIG. 5D).

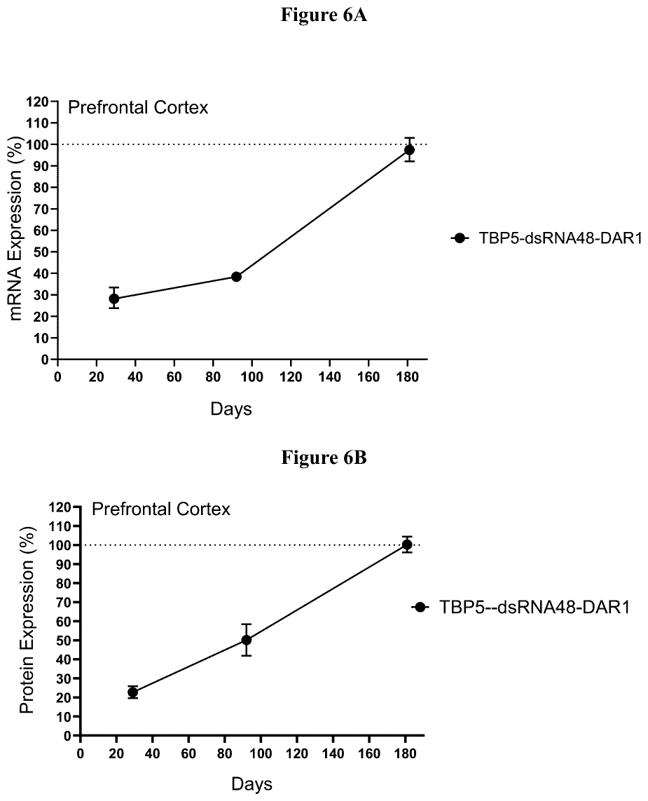

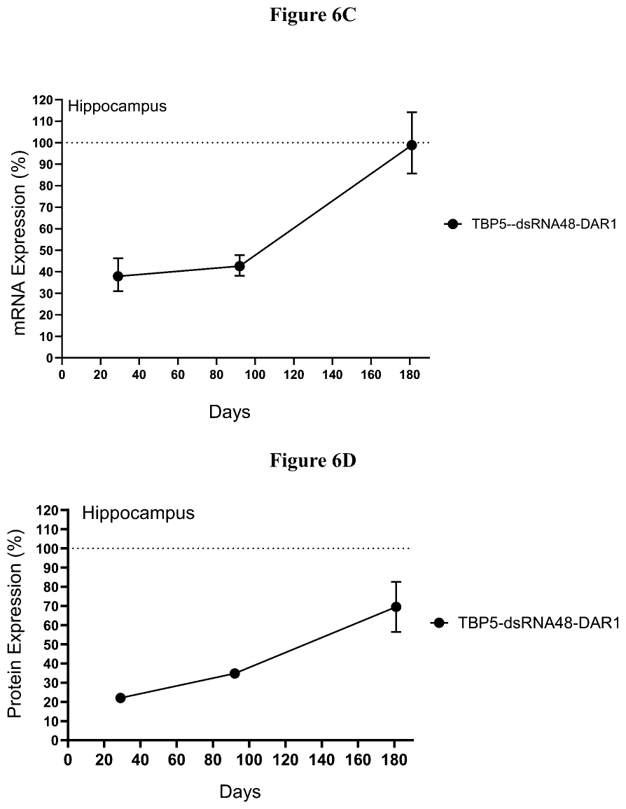

An additional longitudinal study assessing the durability of the mRNA and protein reductions after a single 10 mg/kg IV dose in Cynomolgus monkey (3 animals per group) for human TfR binding proteins-dsRNA conjugate, TBP5-dsRNA No. 48., is presented in FIGS. 6A-D (mean±SEM, n=3). The mRNA and protein reductions were observed to be maximal at the 29 day time-point, but persist out to 92 days. The APP mRNA reduction at 92 days was 62% in prefrontal cortex (FIG. 6A) and 57% in hippocampus (FIG. 6C). The reduction of Aβ(1-x) protein, including Aβ(1-42) and Aβ(1-40), at 92 days was 50% in prefrontal cortex (FIG. 6B) and 65% in hippocampus (FIG. 6D).

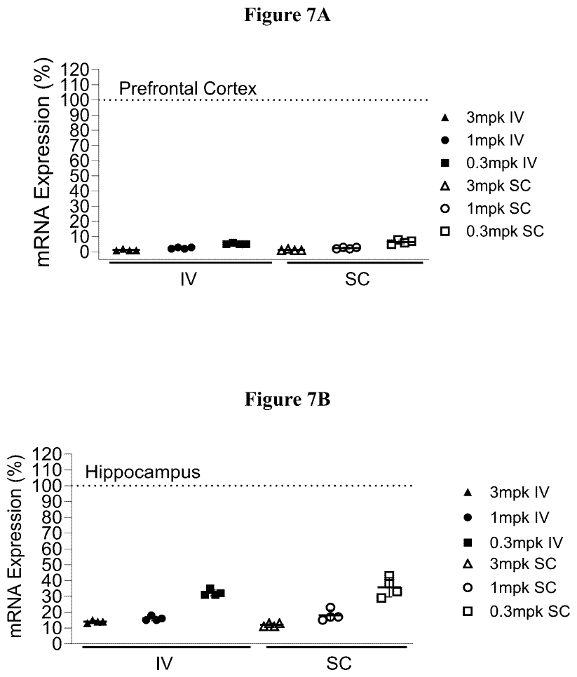

Having demonstrated the potency of the human TfR binding proteins-dsRNA conjugates by intravenous route of administration, the efficacy of TBP5-dsRNA No. 48 (DAR1) conjugate delivered by a single subcutaneous (SC) administration and comparison to IV administration were studied. FIGS. 7A-7B show a head-to-head comparison of a single 3, 1, and 0.3 mg/kg (effective siRNA concentration) dose delivered via either an IV or SC route of administration in hTfR mice. For takedowns, deeply anesthetized animals underwent cardiac perfusion on day 29 and brain tissues were collected and processed for RT-qPCR. FIGS. 7A and 7B show similarly high efficacy of TBP5-dsRNA No. 48 (DAR1) conjugate via either IV or SC delivery at all doses evaluated, with less than 10% APP mRNA remaining in the prefrontal cortex. The dose-response in the hippocampus also showed similar efficacy between the IV and SC administration routes; with 12% APP mRNA remaining at 3 mg/kg dose, 18% remaining at 1 mg/kg dose, and 36% mRNA remaining at 0.3 mg/kg dose for the SC dosed mice. These results support favorable bio-distribution of the TBP5-dsRNA No. 48 (DAR1) conjugate after subcutaneous delivery to disease relevant brain tissues.

### Example 6. Characterization of APP RNAi Agent Comprising dsRNA Sequence with Different Chemical Modifications

Selected APP RNAi agents with different antisense strand modifications (e.g., dsRNA Nos. 107, 108, 109, 110 from Table 7a) were tested in vitro for inhibiting APP expression in EFO-21 cells. EFO-21 cell culture methods are described in Example 4.

Results provided in Table 18 show cholesterol conjugated dsRNA Nos. 107, 108, 109, and 110 successfully reduced APP gene (mRNA) expression in EFO-21 cells and their IC50.

TABLE 18
Characterization of dsRNA with different chemical modifications

| | EFO-21 IC50 (nM) |
|---|---|
| dsRNA No. | Cholesterol-dsRNA |
| 107 | 159.7 |
| 108 | 134.5 |
| 109 | 170.2 |
| 110 | 113.8 |

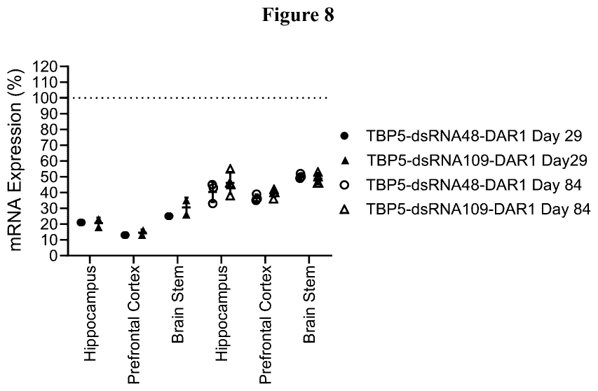

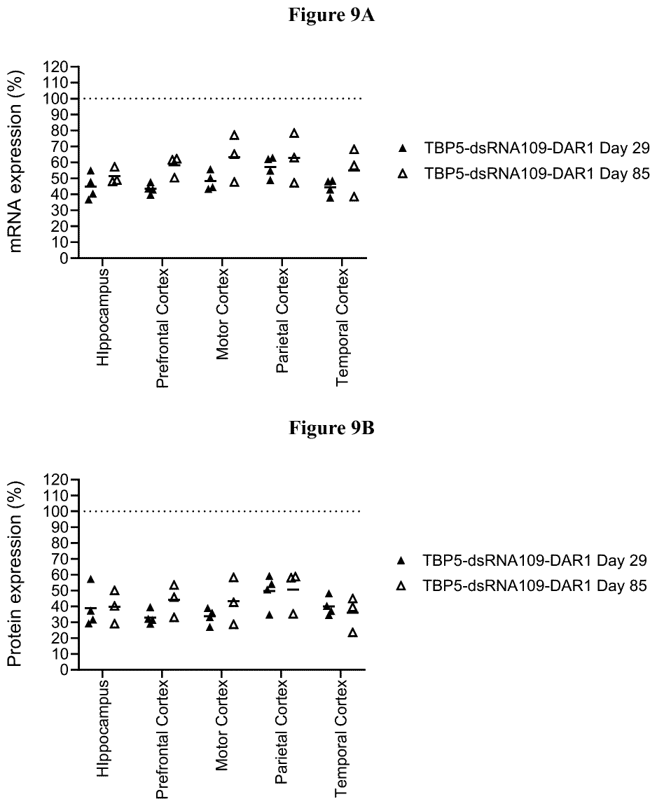

Selected TfR binding protein-dsRNA conjugates with different dsRNA modifications were tested in vivo to assess pharmacodynamic efficacy after peripheral delivery via an intravenous route of delivery in both rodents and non-human primates. More specifically, human TfR transgenic knock-in mice where the extracellular domain of transferrin-receptor has been humanized, received a single 1 mg/kg (dsRNA) IV dose of TBP5-dsRNA No. 48 (DAR1) or TBP5-dsRNA No. 109 (DAR1), or a PBS (phosphate buffered saline) control. Animals were sacrificed 28 or 84 days after injection. Brain samples were collected to assess pharmacodynamic efficacy as shown in FIG. 8. For Cynomolgus monkeys (three-four animals per group) received a single injection of 10 mg/kg (effective dsRNA concentration) in the Saphenous vein of the thigh. The monkeys were injected with either PBS (phosphate buffered saline) or the APP RNAi agent TBP5-dsRNA No. 109 (DAR1) and sacrificed 29 or 85 days after dosing. Perfused brain were coronally sectioned, and punches were collected from subregions including prefrontal cortex, temporal cortex, motor cortex, parietal cortex, hippocampus to assess pharmacodynamic efficacy as shown in FIG. 9. mRNA and protein expression levels were quantified using methods described in Example 5.

FIG. 8 shows APP mRNA reductions in disease relevant hippocampal and cortical regions 28 and 84 days after a single IV dose in hTfR mice of TBP5-dsRNA No. 48 or TBP5-dsRNA No. 109. TBP5-dsRNA No. 48 treatment resulted in APP mRNA reductions of 79% in hippocampus and 87% in prefrontal cortex at 28 days. Further, 3-month durability of TBP5-dsRNA No. 48 treatment was observed with APP mRNA reductions of 60% in hippocampus and 63% in prefrontal cortex. TBP5-dsRNA No. 109 which has an inverted abasic cap to the 3′ end of the antisense strand showed APP mRNA reductions of 80% in hippocampus and 85% in prefrontal cortex at 28 days. TBP5-dsRNA No. 109 also had comparable durability with APP mRNA reductions of 54% in hippocampus and 61% in prefrontal cortex 3-months post dose.

The durability of the mRNA and protein reductions after a single 10 mg/kg IV dose was also performed in a Cynomolgus monkey (3-4 animals per group) for human TfR binding proteins-dsRNA conjugate, TBP5-dsRNA No. 109, is presented in FIGS. 9A-B. The mRNA and protein reductions were observed to be maximal at 29-day time-point but persist out to 92 days across the disease relevant cortical and hippocampal regions. The mean APP mRNA reduction across these regions of interest was 53% (ranging from 42-57%) at 29 days and persisted with a mean reduction of 43% (ranging from 38-49%) at 85 days (FIG. 9A). The mean reduction of Aβ(1-x) protein, including Aβ(1-42) and Aβ(1-40), across these regions of interest was 61% (ranging from 50-67%) at 29 days, and persisted with a mean reduction of 57% (ranging from 49-64%) at 85 days (FIG. 9B). These results demonstrate that modifications such as inclusion of an inverted abasic cap to the 3′ end of the antisense strand, TBP5-dsRNA No. 109, achieves potent and durable knock-down in disease relevant brain tissues.

SEQUENCE LISTING

| SEQ | |
|---|---|
| ID | |
| NO | Sequence |
| 1 | SYSMN |
| 2 | SISSSSSYIYYADSVKG |
| 3 | RHGYSNSDAFDN |
| 4 | RASQGISHYLV |
| 5 | AASSLQS |
| 6 | LQHNSYPWT |
| 7 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSS |
| 8 | DIQMTQSPSAMSASVGDRVTITCRASQGISHYLVWFQQKPGKVPKRLIYAASSLQS |
| | GVPSRFSGSGSGTEFTLTISSLQPEDFATYYCLQHNSYPWTFGQGTKVEIK |
| 9 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKRVEPKC |
| 10 | DIQMTQSPSAMSASVGDRVTITCRASQGISHYLVWFQQKPGKVPKRLIYAASSLQS |
| | GVPSRFSGSGSGTEFTLTISSLQPEDFATYYCLQHNSYPWTFGQGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC |
| 11 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSSASTKGPCVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKRVEPKC |
| | DKTHTGGGGQGGGGQGGGGQGGGGQGGGGQEVQLLESGGGLVQPGGSLRLSCA |
| | ASGRYIDETAVAWFRQAPGKGREFVAGIGGGVDITYYADSVKGRFTISRDNSKNTL |
| | YLQMNSLRPEDTAVYYCGARPGRPLITSKVADLYPYWGQGTLVTVSSPP |
| 12 | DIQMTQSPSAMSASVGDRVTITCRASQGISHYLVWFQQKPGKVPKRLIYAASSLQS |
| | GVPSRFSGSGSGTEFTLTISSLQPEDFATYYCLQHNSYPWTFGQGTKVEIKRTVAAP |
| | SVFIFPPSDEQLKSGTASVVCLLNNFYPREAKVQWKVDNALQCGNSQESVTEQDSK |
| | DSTYSLSSTLTLSKADYEKHKVYACEVTHQGLSSPVTKSFNRGEC |
| 13 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSSASTKGPXVFPLAPCSRSTSESTAALGCLVKDYFPEPVTVSWNSGA |
| | LTSGVHTFPAVLQSSGLYSLSSVVTVPSSSLGTKTYTCNVDHKPSNTKVDKRVESK |
| | YGPPCPPCPAPEAAGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKGLPSSIE |
| | KTISKAKGQPREPQVYTLPPSQEEMTKNQVSLTCLVKGFYPSDIAVEWESNGQPEN |
| | NYKTTPPVLDSDGSFFLYSRLTVDKSRWQEGNVFSCSVMHEALHNHYTQKSLSLSL |
| | G, wherein X is S or C. |
| 14 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSSASTKGPSVFPLAPCSRSTSESTAALGCLVKDYFPEPVTVSWNSGA |
| | LTSGVHTFPAVLQSSGLYSLSSVVTVPSSSLGTKTYTCNVDHKPSNTKVDKRVESK |
| | YGPPCPPCPAPEAAGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKGLPSSIE |
| | KTISKAKGQPREPQVSTLPPSQEEMTKNQVSLMCLVYGFYPSDICVEWESNGQPEN |
| | NYKTTPPVLDSDGSFFLYSVLTVDKSRWQEGNVFSCSVMHEALHNHYTQKSLSLSL |
| | G |
| 15 | ESKYGPPCPPCPAPEAAGGPSVFLFPPKPKDTLMISRTPEVTCVVVDVSQEDPEVQF |
| | NWYVDGVEVHNAKTKPREEQFNSTYRVVSVLTVLHQDWLNGKEYKCKVSNKGL |
| | PSSIEKTISKAKGQPREPQVYTLPPSQGDMTKNQVQLTCLVKGFYPSDICVEWESNG |
| | QPENNYKTTPPVLDSDGSFFLASRLTVDKSRWQEGNVFSCSVMHEALHNHYTQKS |
| | LSLSLG |
| 16 | EVQLVESGGGLVKPGGSLRLSCVASGFTFSSYSMNWVRQAPGKGLEWVSSISSSSS |
| | YIYYADSVKGRFTISRDNAKNSLYLQMNSLRAEDTAVYYCARRHGYSNSDAFDN |
| | WGQGTLVTVSSASTKGPCVFPLAPCSRSTSESTAALGCLVKDYFPEPVTVSWNSGA |
| | LTSGVHTFPAVLQSSGLYSLSSVVTVPSSSLGTKTYTCNVDHKPSNTKVDKRVESK |
| | YGPPCPPCPAPEAAGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKGLPSSIE |
| | KTISKAKGQPREPQVSTLPPSQEEMTKNQVSLMCLVYGFYPSDIAVEWESNGQPEN |
| | NYKTTPPVLDSDGSFFLYSVLTVDKSRWQEGNVFSCSVMHEALHNHYTQKSLSLSL |
| | G |
| 17 | ESKYGPPCPPCPAPEAAGGPSVFLFPPKPKDTLMISRTPEVTCVVVDVSQEDPEVQF |
| | NWYVDGVEVHNAKTKPREEQFNSTYRVVSVLTVLHQDWLNGKEYKCKVSNKGL |
| | PSSIEKTISKAKGQPREPQVYTLPPSQGDMTKNQVQLTCLVKGFYPSDIAVEWESNG |
| | QPENNYKTTPPVLDSDGSFFLASRLTVDKSRWQEGNVFSCSVMHEALHNHYTQKS |
| | LSLSLG |
| 18 | QVQLVQSGAEVKKPGSSVKVSCKASGYTFSSYAIEWVRQAPGQGLEWMGGILPGS |
| | GTINYNEKFKGRVTITADKSTSTAYMELSSLRSEDTAVYYCARMSSNSDQGFDLW |
| | GQGTLVTVSSASTKGPXVFPLAPCSRSTSESTAALGCLVKDYFPEPVTVSWNSGAL |
| | TSGVHTFPAVLQSSGLYSLSSVVTVPSSSLGTKTYTCNVDHKPSNTKVDKRVESKY |
| | GPPCPPCPAPEAAGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSNKGLPSSIEK |
| | TISKAKGQPREPQVYTLPPSQEEMTKNQVSLTCLVKGFYPSDIAVEWESNGQPENN |
| | YKTTPPVLDSDGSFLLYSKLTVDKSRWQEGNVFSCSVMHEALHNHYTQKSLSLSL |
| | G, wherein X is S or C. |
| 19 | DIQMTQSPSSLSASVGDRVTITCKASQGISRFLSWFQQKPGKAPKSLIYAVSSLVDG |
| | VPSRFSGSGSGTDFTLTISSLQPEDFATYYCVQYNSYPYGFGGGTKVEIKRTVAAPS |
| | VFIFPPSDEQLKSGTASVVCLLNNFYPREAKVQWKVDNALQSGNSQESVTEQDSKD |
| | STYSLSSTLTLSKADYEKHKVYACEVTHQGLSSPVTKSFNRGEC |
| 20 | ETAVA |
| 21 | GIGGGVDITYYADSVKG |
| 22 | RPGRPLITSKVADLYPY |
| 23 | EVQLLESGGGLVQPGGSLRLSCAASGRYIDETAVAWFRQAPGKGREFVAGIGGGV |
| | DITYYADSVKGRFTISRDNSKNTLYLQMNSLRPEDTAVYYCGARPGRPLITSKVAD |
| | LYPYWGQGTLVTVSSPP |
| 24 | GGGGQGGGGQGGGGQGGGGQ |
| 25 | GSYWIC |
| 26 | CIYSTSGGRTYYASWVKG |
| 27 | GDDSISDAYFDL |
| 28 | QSSQSVYNNNRLA |
| 29 | DASTLAS |
| 30 | QGTYFSSGWSWA |
| 31 | QSLEESGGDLVKPEGSLTLTCTASGFSFSGSYWICWVRQAPGKGLEWIGCIYSTSGG |
| | RTYYASWVKGRFTISKTSSTTVTLQMTSLTAADTATYFCARGDDSISDAYFDLWGP |
| | GTLVTVSS |
| 32 | ALDMTQTASPVSAAVGGTVTINCQSSQSVYNNNRLAWYQQKPGQPPKLLIYDAST |
| | LASGVPSRFKGSGSGTQFTLTISGVQSDDSATYYCQGTYFSSGWSWAFGGGTEVVV |
| | K |
| 33 | QSLEESGGDLVKPEGSLTLTCTASGFSFSGSYWICWVRQAPGKGLEWIGCIYSTSGG |
| | RTYYASWVKGRFTISKTSSTTVTLQMTSLTAADTATYFCARGDDSISDAYFDLWGP |
| | GTLVTVSSASTKGPCVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPP |
| | CPPCPAPEAAGGPSVFLFPPKPKDTLMISRTPEVTCVVVDVSQEDPEVQFNWYVDG |
| | VEVHNAKTKPREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWESNGQPENNYK |
| | TTPPVLDSDGSFFLYSRLTVDKSRWQEGNVFSCSVMHEALHNHYTQKSLSLSLG |
| 34 | ALDMTQTASPVSAAVGGTVTINCQSSQSVYNNNRLAWYQQKPGQPPKLLIYDAST |
| | LASGVPSRFKGSGSGTQFTLTISGVQSDDSATYYCQGTYFSSGWSWAFGGGTEVVV |
| | KRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 35 | AGCAAAACCAUUGCUUCACUA |
| 36 | UAGUGAAGCAAUGGUUUUGCUGU |
| 37 | CAGCAAAACCAUUGCUUCACA |
| 38 | UGUGAAGCAAUGGUUUUGCUGUC |
| 39 | GAUGCCUGAACUUGAAUUAAA |
| 40 | UUUAAUUCAAGUUCAGGCAUCUA |
| 41 | CUGUAACACAAGUAGAUGCCA |
| 42 | UGGCAUCUACUUGUGUUACAGCA |
| 43 | CCUGAUCACUAUGCAUUUUAA |
| 44 | UUAAAAUGCAUAGUGAUCAGGAA |
| 45 | UGACAGCUGUGCUGUAACACA |
| 46 | UGUGUUACAGCACAGCUGUCAAA |
| 47 | CAUGAAUAGAUUCUCUCCUGA |
| 48 | UCAGGAGAGAAUCUAUUCAUGCA |
| 49 | GUACAGAAUCAUUGCUUAUGA |
| 50 | UCAUAAGCAAUGAUUCUGUACAA |
| 51 | UGUCCACGUAUCUUUGGGUCA |
| 52 | UGACCCAAAGAUACGUGGACAAA |
| 53 | GUCCACGUAUCUUUGGGUCUA |
| 54 | UAGACCCAAAGAUACGUGGACAA |
| 55 | GAUAUAGGAAUUAAGAGGAUA |
| 56 | UAUCCUCUUAAUUCCUAUAUCAC |
| 57 | ACAGAUUGCUGCUUCUGCUAA |
| 58 | UUAGCAGAAGCAGCAAUCUGUAC |
| 59 | CCAAGAUGCAGCAGAACGGCA |
| 60 | UGCCGUUCUGCUGCAUCUUGGAC |
| 61 | AGGAAGCAGCCAACGAGAGAA |
| 62 | UUCUCUCGUUGGCUGCUUCCUGU |
| 63 | CUUUGAGCAGAUGCAGAACUA |
| 64 | UAGUUCUGCAUCUGCUCAAAGAA |
| 65 | GGCAGUUAUCCAGCAUUUCCA |
| 66 | UGGAAAUGCUGGAUAACUGCCUU |
| 67 | UCCAACCUACAAGUUCUUUGA |
| 68 | UCAAAGAACUUGUAGGUUGGAUU |
| 69 | CCAACCUACAAGUUCUUUGAA |
| 70 | UUCAAAGAACUUGUAGGUUGGAU |
| 71 | CUGAAGAAGAAACAGUACACA |
| 72 | UGUGUACUGUUUCUUCUUCAGCA |
| 73 | GACAAAGUAGUAGAAGUAGCA |
| 74 | UGCUACUUCUACUACUUUGUCUU |
| 75 | AAGUUCUUUGAGCAGAUGCAA |
| 76 | UUGCAUCUGCUCAAAGAACUUGU |
| 77 | AGGCAGUUAUCCAGCAUUUCA |
| 78 | UGAAAUGCUGGAUAACUGCCUUC |
| 79 | AGCACCGAGAGAGAAUGUCCA |
| 80 | UGGACAUUCUCUCUCGGUGCUUG |
| 81 | CCGGUCCCAGGUUAUGACACA |
| 82 | UGUGUCAUAACCUGGGACCGGAU |
| 83 | AACCAGUGACCAUCCAGAACA |
| 84 | UGUUCUGGAUGGUCACUGGUUGG |
| 85 | ACCGAGGACUGACCACUCGAA |
| 86 | UUCGAGUGGUCAGUCCUCGGUCG |
| 87 | CACACCGUCGCCAAAGAGACA |
| 88 | UGUCUCUUUGGCGACGGUGUGCC |
| 89 | GCCAAGCACCGAGAGAGAAUA |
| 90 | UAUUCUCUCUCGGUGCUUGGCCU |
| 91 | GAUCCGGUCCCAGGUUAUGAA |
| 92 | UUCAUAACCUGGGACCGGAUCUG |
| 93 | AAGCACCGAGAGAGAAUGUCA |
| 94 | UGACAUUCUCUCUCGGUGCUUGG |
| 95 | CCAUCCAGAACUGGUGCAAGA |
| 96 | UCUUGCACCAGUUCUGGAUGGUC |
| 97 | GUCCAAGAUGCAGCAGAACGA |
| 98 | UCGUUCUGCUGCAUCUUGGACAG |
| 99 | CAGGUCAUGAGAGAAUGGGAA |
| 100 | UUCCCAUUCUCUCAUGACCUGGG |
| 101 | mA*mG*mCmAmAmAfAmCfCfAfUmUmGmCmUmUmCmAmC*mU*mA |
| 102 | mU*fA*mGmUmGfAmAmGmCmAmAmUmGfGmUfUmUmUmGmCmU*mG*mU |
| 103 | mC*mA*mGmCmAmAfAmAfCfCfAmUmUmGmCmUmUmCmA*mC*mA |
| 104 | mU*fG*mUmGmAfAmGmCmAmAmUmGmGfUmUfUmUmGmCmUmG*mU*mC |
| 105 | mG*mA*mUmGmCmCfUmGfAfAfCmUmUmGmAmAmUmUmA*mA*mA |
| 106 | mU*fU*mUmAmAfUmUmCmAmAmGmUmUfCmAfGmGmCmAmUmC*mU*mA |
| 107 | mC*mU*mGmUmAmAfCmAfCfAfAmGmUmAmGmAmUmGmC*mC*mA |
| 108 | mU*fG*mGmCmAfUmCmUmAmCmUmUmGfUmGfUmUmAmCmAmG*mC*mA |
| 109 | mC*mC*mUmGmAmUfCmAfCfUfAmUmGmCmAmUmUmUmU*mA*mA |
| 110 | mU*fU*mAmAmAfAmUmGmCmAmUmAmGfUmGfAmUmCmAmGmG*mA*mA |
| 111 | mU*mG*mAmCmAmGfCmUfGfUfGmCmUmGmUmAmAmCmA*mC*mA |
| 112 | mU*fG*mUmGmUfUmAmCmAmGmCmAmCfAmGfCmUmGmUmCmA*mA*mA |
| 113 | mC*mA*mUmGmAmAfUmAfGfAfUmUmCmUmCmUmCmCmU*mG*mA |
| 114 | mU*fC*mAmGmGfAmGmAmGmAmAmUmCfUmAfUmUmCmAmUmG*mC*mA |
| 115 | mG*mU*mAmCmAmGfAmAfUfCfAmUmUmGmCmUmUmAmU*mG*mA |
| 116 | mU*fC*mAmUmAfAmGmCmAmAmUmGmAfUmUfCmUmGmUmAmC*mA*mA |
| 117 | mU*mG*mUmCmCmAfCmGfUfAfUmCmUmUmUmGmGmGmU*mC*mA |
| 118 | mU*fG*mAmCfCmCfAmAmAmGmAmUmAfCmGfUmGmGmAmCmA*mA*mA |
| 119 | mG*mU*mCmCmAmCfGmUfAfUfCmUmUmUmGmGmGmUmC*mU*mA |
| 120 | mU*fA*mGmAfCmCfCmAmAmAmGmAmUfAmCfGmUmGmGmAmC*mA*mA |
| 121 | mG*mA*mUmAmUmAfGmGfAfAfUmUmAmAmGmAmGmGmA*mU*mA |
| 122 | mU*fA*mUmCfCmUfCmUmUmAmAmUmUfCmCfUmAmUmAmUmC*mA*mC |
| 123 | mA*mC*mAmGmAmUfUmGfCfUfGmCmUmUmCmUmGmCmU*mA*mA |
| 124 | mU*fU*mAmGfCmAfGmAmAmGmCmAmGfCmAfAmUmCmUmGmU*mA*mC |
| 125 | mC*mC*mAmAmGmAfUmGfCfAfGmCmAmGmAmAmCmGmG*mC*mA |
| 126 | mU*fG*mCmCmGfUmUmCmUmGmCmUmGfCmAfUmCmUmUmGmG*mA*mC |
| 127 | mA*mG*mGmAmAmGfCmAfGfCfCmAmAmCmGmAmGmAmG*mA*mA |
| 128 | mU*fU*mCmUmCfUmCmGmUmUmGmGmCfUmGfCmUmUmCmCmU*mG*mU |
| 129 | mC*mU*mUmUmGmAfGmCfAfGfAmUmGmCmAmGmAmAmC*mU*mA |
| 130 | mU*fA*mGmUmUfCmUmGmCmAmUmCmUfGmCfUmCmAmAmAmG*mA*mA |
| 131 | mG*mG*mCmAmGmUfUmAfUfCfCmAmGmCmAmUmUmUmC*mC*mA |
| 132 | mU*fG*mGmAmAfAmUmGmCmUmGmGmAfUmAfAmCmUmGmCmC*mU*mU |
| 133 | mU*mC*mCmAmAmCfCmUfAfCfAmAmGmUmUmCmUmUmU*mG*mA |
| 134 | mU*fC*mAmAmAfGmAmAmCmUmUmGmUfAmGfGmUmUmGmGmA*mU*mU |
| 135 | mC*mC*mAmAmCmCfUmAfCfAfAmGmUmUmCmUmUmUmG*mA*mA |
| 136 | mU*fU*mCmAmAfAmGmAmAmCmUmUmGfUmAfGmGmUmUmGmG*mA*mU |
| 137 | mC*mU*mGmAmAmGfAmAfGfAfAmAmCmAmGmUmAmCmA*mC*mA |
| 138 | mU*fG*mUmGmUfAmCmUmGmUmUmUmCfUmUfCmUmUmCmAmG*mC*mA |
| 139 | mG*mA*mCmAmAmAfGmUfAfGfUmAmGmAmAmGmUmAmG*mC*mA |
| 140 | mU*fG*mCmUmAfCmUmUmCmUmAmCmUfAmCfUmUmUmGmUmC*mU*mU |
| 141 | mA*mA*mGmUmUmCfUmUfUfGfAmGmCmAmGmAmUmGmC*mA*mA |
| 142 | mU*fU*mGmCmAfUmCmUmGmCmUmCmAfAmAfGmAmAmCmUmU*mG*mU |
| 143 | mA*mG*mGmCmAmGfUmUfAfUfCmCmAmGmCmAmUmUmU*mC*mA |
| 144 | mU*fG*mAmAmAfUmGmCmUmGmGmAmUfAmAfCmUmGmCmCmU*mU*mC |
| 145 | mA*mG*mCmAmCmCfGmAfGfAfGmAmGmAmAmUmGmUmC*mC*mA |
| 146 | mU*fG*mGmAmCfAmUmUmCmUmCmUmCfUmCfGmGmUmGmCmU*mU*mG |
| 147 | mC*mC*mGmGmUmCfCmCfAfGfGmUmUmAmUmGmAmCmA*mC*mA |
| 148 | mU*fG*mUmGmUfCmAmUmAmAmCmCmUfGmGfGmAmCmCmGmG*mA*mU |
| 149 | mA*mA*mCmCmAmGfUmGfAfCfCmAmUmCmCmAmGmAmA*mC*mA |
| 150 | mU*fG*mUmUmCfUmGmGmAmUmGmGmUfCmAfCmUmGmGmUmU*mG*mG |
| 151 | mA*mC*mCmGmAmGfGmAfCfUfGmAmCmCmAmCmUmCmG*mA*mA |
| 152 | mU*fU*mCmGmAfGmUmGmGmUmCmAmGfUmCfCmUmCmGmGmU*mC*mG |
| 153 | mC*mA*mCmAmCmCfGmUfCfGfCmCmAmAmAmGmAmGmA*mC*mA |
| 154 | mU*fG*mUmCmUfCmUmUmUmGmGmCmGfAmCfGmGmUmGmUmG*mC*mC |
| 155 | mG*mC*mCmAmAmGfCmAfCfCfGmAmGmAmGmAmGmAmA*mU*mA |
| 156 | mU*fA*mUmUmCfUmCmUmCmUmCmGmGfUmGfCmUmUmGmGmC*mC*mU |
| 157 | mG*mA*mUmCmCmGfGmUfCfCfCmAmGmGmUmUmAmUmG*mA*mA |
| 158 | mU*fU*mCmAmUfAmAmCmCmUmGmGmGfAmCfCmGmGmAmUmC*mU*mG |
| 159 | mA*mA*mGmCmAmCfCmGfAfGfAmGmAmGmAmAmUmGmU*mC*mA |
| 160 | mU*fG*mAmCmAfUmUmCmUmCmUmCmUfCmGfGmUmGmCmUmU*mG*mG |
| 161 | mC*mC*mAmUmCmCfAmGfAfAfCmUmGmGmUmGmCmAmA*mG*mA |
| 162 | mU*fC*mUmUmGfCmAmCmCmAmGmUmUfCmUfGmGmAmUmGmG*mU*mC |
| 163 | mG*mU*mCmCmAmAfGmAfUfGfCmAmGmCmAmGmAmAmC*mG*mA |
| 164 | mU*fC*mGmUmUfCmUmGmCmUmGmCmAfUmCfUmUmGmGmAmC*mA*mG |
| 165 | mC*mA*mGmGmUmCfAmUfGfAfGmAmGmAmAmUmGmGmG*mA*mA |
| 166 | mU*fU*mCmCmCfAmUmUmCmUmCmUmCfAmUfGmAmCmCmUmG*mG*mG |
| 167 | MLPGLALLLLAAWTARALEVPTDGNAGLLAEPQIAMFCGRLNMHMNVQNGKWD |
| | SDPSGTKTCIDTKEGILQYCQEVYPELQITNVVEANQPVTIQNWCKRGRKQCKTHP |
| | HFVIPYRCLVGEFVSDALLVPDKCKFLHQERMDVCETHLHWHTVAKETCSEKSTN |
| | LHDYGMLLPCGIDKFRGVEFVCCPLAEESDNVDSADAEEDDSDVWWGGADTDYA |
| | DGSEDKVVEVAEEEEVAEVEEEEADDDEDDEDGDEVEEEAEEPYEEATERTTSIAT |
| | TTTTTTESVEEVVREVCSEQAETGPCRAMISRWYFDVTEGKCAPFFYGGCGGNRN |
| | NFDTEEYCMAVCGSAMSQSLLKTTQEPLARDPVKLPTTAASTPDAVDKYLETPGD |
| | ENEHAHFQKAKERLEAKHRERMSQVMREWEEAERQAKNLPKADKKAVIQHFQEK |
| | VESLEQEAANERQQLVETHMARVEAMLNDRRRLALENYITALQAVPPRPRHVFNM |
| | LKKYVRAEQKDRQHTLKHFEHVRMVDPKKAAQIRSQVMTHLRVIYERMNQSLSL |
| | LYNVPAVAEEIQDEVDELLQKEQNYSDDVLANMISEPRISYGNDALMPSLTETKTT |
| | VELLPVNGEFSLDDLQPWHSFGADSVPANTENEVEPVDARPAADRGLTTRPGSGLT |
| | NIKTEEISEVKMDAEFRHDSGYEVHHQKLVFFAEDVGSNKGAIIGLMVGGVVIATV |
| | IVITLVMLKKKQYTSIHHGVVEVDAAVTPEERHLSKMQQNGYENPTYKFFEQMQN |
| 168 | GTCAGTTTCCTCGGCAGCGGTAGGCGAGAGCACGCGGAGGAGCGTGCGCGGGGGCCCC |
| | GGGAGACGGCGGCGGTGGCGGCGCGGGCAGAGCAAGGACGCGGCGGATCCCACTCGC |
| | ACAGCAGCGCACTCGGTGCCCCGCGCAGGGTCGCGATGCTGCCCGGTTTGGCACTGCTC |
| | CTGCTGGCCGCCTGGACGGCTCGGGCGCTGGAGGTACCCACTGATGGTAATGCTGGCCT |
| | GCTGGCTGAACCCCAGATTGCCATGTTCTGTGGCAGACTGAACATGCACATGAATGTCC |
| | AGAATGGGAAGTGGGATTCAGATCCATCAGGGACCAAAACCTGCATTGATACCAAGGA |
| | AGGCATCCTGCAGTATTGCCAAGAAGTCTACCCTGAACTGCAGATCACCAATGTGGTAG |
| | AAGCCAACCAACCAGTGACCATCCAGAACTGGTGCAAGCGGGGCCGCAAGCAGTGCAA |
| | GACCCATCCCCACTTTGTGATTCCCTACCGCTGCTTAGTTGGTGAGTTTGTAAGTGATGC |
| | CCTTCTCGTTCCTGACAAGTGCAAATTCTTACACCAGGAGAGGATGGATGTTTGCGAAA |
| | CTCATCTTCACTGGCACACCGTCGCCAAAGAGACATGCAGTGAGAAGAGTACCAACTT |
| | GCATGACTACGGCATGTTGCTGCCCTGCGGAATTGACAAGTTCCGAGGGGTAGAGTTTG |
| | TGTGTTGCCCACTGGCTGAAGAAAGTGACAATGTGGATTCTGCTGATGCGGAGGAGGA |
| | TGACTCGGATGTCTGGTGGGGCGGAGCAGACACAGACTATGCAGATGGGAGTGAAGAC |
| | AAAGTAGTAGAAGTAGCAGAGGAGGAAGAAGTGGCTGAGGTGGAAGAAGAAGAAGCC |
| | GATGATGACGAGGACGATGAGGATGGTGATGAGGTAGAGGAAGAGGCTGAGGAACCC |
| | TACGAAGAAGCCACAGAGAGAACCACCAGCATTGCCACCACCACCACCACCACCACAG |
| | AGTCTGTGGAAGAGGTGGTTCGAGAGGTGTGCTCTGAACAAGCCGAGACGGGGCCGTG |
| | CCGAGCAATGATCTCCCGCTGGTACTTTGATGTGACTGAAGGGAAGTGTGCCCCATTCT |
| | TTTACGGCGGATGTGGCGGCAACCGGAACAACTTTGACACAGAAGAGTACTGCATGGC |
| | CGTGTGTGGCAGCGCCATGTCCCAAAGTTTACTCAAGACTACCCAGGAACCTCTTGCCC |
| | GAGATCCTGTTAAACTTCCTACAACAGCAGCCAGTACCCCTGATGCCGTTGACAAGTAT |
| | CTCGAGACACCTGGGGATGAGAATGAACATGCCCATTTCCAGAAAGCCAAAGAGAGGC |
| | TTGAGGCCAAGCACCGAGAGAGAATGTCCCAGGTCATGAGAGAATGGGAAGAGGCAG |
| | AACGTCAAGCAAAGAACTTGCCTAAAGCTGATAAGAAGGCAGTTATCCAGCATTTCCA |
| | GGAGAAAGTGGAATCTTTGGAACAGGAAGCAGCCAACGAGAGACAGCAGCTGGTGGA |
| | GACACACATGGCCAGAGTGGAAGCCATGCTCAATGACCGCCGCCGCCTGGCCCTGGAG |
| | AACTACATCACCGCTCTGCAGGCTGTTCCTCCTCGGCCTCGTCACGTGTTCAATATGCTA |
| | AAGAAGTATGTCCGCGCAGAACAGAAGGACAGACAGCACACCCTAAAGCATTTCGAGC |
| | ATGTGCGCATGGTGGATCCCAAGAAAGCCGCTCAGATCCGGTCCCAGGTTATGACACA |
| | CCTCCGTGTGATTTATGAGCGCATGAATCAGTCTCTCTCCCTGCTCTACAACGTGCCTGC |
| | AGTGGCCGAGGAGATTCAGGATGAAGTTGATGAGCTGCTTCAGAAAGAGCAAAACTAT |
| | TCAGATGACGTCTTGGCCAACATGATTAGTGAACCAAGGATCAGTTACGGAAACGATG |
| | CTCTCATGCCATCTTTGACCGAAACGAAAACCACCGTGGAGCTCCTTCCCGTGAATGGA |
| | GAGTTCAGCCTGGACGATCTCCAGCCGTGGCATTCTTTTGGGGCTGACTCTGTGCCAGC |
| | CAACACAGAAAACGAAGTTGAGCCTGTTGATGCCCGCCCTGCTGCCGACCGAGGACTG |
| | ACCACTCGACCAGGTTCTGGGTTGACAAATATCAAGACGGAGGAGATCTCTGAAGTGA |
| | AGATGGATGCAGAATTCCGACATGACTCAGGATATGAAGTTCATCATCAAAAATTGGT |
| | GTTCTTTGCAGAAGATGTGGGTTCAAACAAAGGTGCAATCATTGGACTCATGGTGGGCG |
| | GTGTTGTCATAGCGACAGTGATCGTCATCACCTTGGTGATGCTGAAGAAGAAACAGTAC |
| | ACATCCATTCATCATGGTGTGGTGGAGGTTGACGCCGCTGTCACCCCAGAGGAGCGCCA |
| | CCTGTCCAAGATGCAGCAGAACGGCTACGAAAATCCAACCTACAAGTTCTTTGAGCAG |
| | ATGCAGAACTAGACCCCCGCCACAGCAGCCTCTGAAGTTGGACAGCAAAACCATTGCT |
| | TCACTACCCATCGGTGTCCATTTATAGAATAATGTGGGAAGAAACAAACCCGTTTTATG |
| | ATTTACTCATTATCGCCTTTTGACAGCTGTGCTGTAACACAAGTAGATGCCTGAACTTG |
| | AATTAATCCACACATCAGTAATGTATTCTATCTCTCTTTACATTTTGGTCTCTATACTAC |
| | ATTATTAATGGGTTTTGTGTACTGTAAAGAATTTAGCTGTATCAAACTAGTGCATGAAT |
| | AGATTCTCTCCTGATTATTTATCACATAGCCCCTTAGCCAGTTGTATATTATTCTTGTGG |
| | TTTGTGACCCAATTAAGTCCTACTTTACATATGCTTTAAGAATCGATGGGGGATGCTTC |
| | ATGTGAACGTGGGAGTTCAGCTGCTTCTCTTGCCTAAGTATTCCTTTCCTGATCACTATG |
| | CATTTTAAAGTTAAACATTTTTAAGTATTTCAGATGCTTTAGAGAGATTTTTTTTCCATG |
| | ACTGCATTTTACTGTACAGATTGCTGCTTCTGCTATATTTGTGATATAGGAATTAAGAGG |
| | ATACACACGTTTGTTTCTTCGTGCCTGTTTTATGTGCACACATTAGGCATTGAGACTTCA |
| | AGCTTTTCTTTTTTTGTCCACGTATCTTTGGGTCTTTGATAAAGAAAAGAATCCCTGTTC |
| | ATTGTAAGCACTTTTACGGGGCGGGTGGGGAGGGGTGCTCTGCTGGTCTTCAATTACCA |
| | AGAATTCTCCAAAACAATTTTCTGCAGGATGATTGTACAGAATCATTGCTTATGACATG |
| | ATCGCTTTCTACACTGTATTACATAAATAAATTAAATAAAATAACCCCGGGCAAGACTT |
| | TTCTTTGAAGGATGACTACAGACATTAAATAATCGAAGTAATTTTGGGTGGGGAGAAG |
| | AGGCAGATTCAATTTTCTTTAACCAGTCTGAAGTTTCATTTATGATACAAAAGAAGATG |
| | AAAATGGAAGTGGCAATATAAGGGGATGAGGAAGGCATGCCTGGACAAACCCTTCTTT |
| | TAAGATGTGTCTTCAATTTGTATAAAATGGTGTTTTCATGTAAATAAATACATTCTTGGA |
| | GGAGCA |
| 169 | HHHHHHCKRVEQKEECVKLAETEETDKSETMETEDVPTSSRLYWADLKTLLSEKLNSIEFA |
| | DTIKQLSQNTYTPREAGSQKDESLAYYIENQFHEFKFSKVWRDEHYVKIQVKSSIGQNMVTI |
| | VQSNGNLDPVESPEGYVAFSKPTEVSGKLVHANFGTKKDFEELSYSVNGSLVIVRAGEITFA |
| | EKVANAQSFNAIGVLIYMDKNKFPVVEADLALFGHAHLGTGDPYTPGFPSFNHTQFPPSQS |
| | SGLPNIPVQTISRAAAEKLFGKMEGSCPARWNIDSSCKLELSQNQNVKLIVKNVLKERRILNI |
| | FGVIKGYEEPDRYVVVGAQRDALGAGVAAKSSVGTGLLLKLAQVFSDMISKDGFRPSRSIIF |
| | ASWTAGDFGAVGATEWLEGYLSSLHLKAFTYINLDKVVLGTSNFKVSASPLLYTLMGKIM |
| | QDVKHPVDGKSLYRDSNWISKVEKLSFDNAAYPFLAYSGIPAVSFCFCEDADYPYLGTRLD |
| | TYEALTQKVPQLNQMVRTAAEVAGQLIIKLTHDVELNLDYEMYNSKLLSFMKDLNQFKTD |
| | IRDMGLSLQWLYSARGDYFRATSRLTTDFHNAEKTNRFVMREINDRIMKVEYHFLSPYVSP |
| | RESPFRHIFWGSGSHTLSALVENLKLRQKNITAFNETLFRNQLALATWTIQGVANALSGDIW |
| | NIDNEF |
| 170 | HHHHHHCKGVEPKTECERLAGTESPVREEPGEDFPAARRLYWDDLKRKLSEKLDSTDFTG |
| | TIKLLNENSYVPREAGSQKDENLALYVENQFREFKLSKVWRDQHFVKIQVKDSAQNSVIIV |
| | DKNGRLVYLVENPGGYVAYSKAATVTGKLVHANFGTKKDFEDLYTPVNGSIVIVRAGKIT |
| | FAEKVANAESLNAIGVLIYMDQTKFPIVNAELSFFGHAHLGTGDPYTPGFPSFNHTQFPPSRS |
| | SGLPNIPVQTISRAAAEKLFGNMEGDCPSDWKTDSTCRMVTSESKNVKLTVSNVLKEIKILN |
| | IFGVIKGFVEPDHYVVVGAQRDAWGPGAAKSGVGTALLLKLAQMFSDMVLKDGFQPSRSII |
| | FASWSAGDFGSVGATEWLEGYLSSLHLKAFTYINLDKAVLGTSNFKVSASPLLYTLIEKTM |
| | QNVKHPVTGQFLYQDSNWASKVEKLTLDNAAFPFLAYSGIPAVSFCFCEDTDYPYLGTTM |
| | DTYKELIERIPELNKVARAAAEVAGQFVIKLTHDVELNLDYERYNSQLLSFVRDLNQYRADI |
| | KEMGLSLQWLYSARGDFFRATSRLTTDFGNAEKTDRFVMKKLNDRVMRVEYHFLSPYVSP |
| | KESPFRHVFWGSGSHTLPALLENLKLRKQNNGAFNETLFRNQLALATWTIQGAANALSGD |
| | VWDIDNEF |
| 171 | HHHHHHHHGKPIPNPLLGLDSTGGGGSDSAQNSVIIVDKNGRLVYLVENPGGYVAYSKAA |
| | TVTGKLVHANFGTKKDFEDLYTPVNGSIVIVRAGKITFAEKVANAESLNAIGVLIYMDQTKF |
| | PIVNAELSFFGHAHLGGGGGGLPNIPVQTISRAAAEKLFGNMEGDCPSDWKTDSTCRMVTS |
| | ESKNVKLTVS |
| 172 | mA*mG*mCmAmAmAmAmCfCfAfUmUmGmCmUmUmCmAmC*mU*mA |
| 173 | mU*fA*mGmUfGmAfAmGmCmAmAmUmGfGmUfUmUmUmGmCmU*mG*mU |
| 174 | mC*mA*mGmCmAmAmAmAfCfCfAmUmUmGmCmUmUmCmA*mC*mA |
| 175 | mU*fG*mUmGfAmAfGmCmAmAmUmGmGfUmUfUmUmGmCmUmG*mU*mC |
| 176 | mU*fA*mGmUfGmAmAmGmCmAmAmUmGfGmUfUmUmUmGmCmU*mG*mU |
| 177 | mU*fA*mGmUfGmAmAfGmCmAmAmUmGfGmUfUmUmUmGmCmU*mG*mU |
| 178 | mU*fA*mGfUmGmAmAmGmCmAmAmUmGfGmUfUmUmUmGmCmU*mG*mU |
| 179 | mU*fA*mGmUmGmAmAmGmCmAmAmUmGfGmUfUmUmUmGmCmU*mG*mU |
| 180 | mU*fA*fGmUmGmAfAmGmCmAmAmUmGfGmUfUmUmUmGmCmU*mG*mU |
| 181 | mA*mG*mCmAmAmAAmCfCfAfUmUmGmCmUmUmCmAmC*mU*mA |
| 182 | mU*fA*mGfUmGfAfAmGfCmAmAmUmGfGmUfUmUmUmGmCmU*mG*mU |
| 183 | mA*mG*mCmAmAmAfAmCfC(n)fUmUmGmCmUmUmCmAmC*mU*mA |
| 184 | AGCAAAACC(n)UUGCUUCACUA, wherein n is an abasic moiety. |
| 185 | mU*fA*mGmUfGfAfAfGmCmAmAmUmGfGmUfUmUmUmGmCmU*mG*mU |
| 186 | mA*mG*mCmAmAmAfAmCfCdAfUmUmGmCmUmUmCmAmC*mU*mA |
| 187 | mC*mA*mGmCmAmAfAmAfC(n)fAmUmUmGmCmUmUmCmA*mC*mA |
| 188 | CAGCAAAAC(n)AUUGCUUCACA, wherein n is an abasic moiety. |
| 189 | mU*fG*mUmGfAmAmGfCmAmAmUmGmGfUmUfUmUmGmCmUmG*mU*mC |
| 190 | mU*fG*mUfGmAmAmGmCmAmAmUmGmGfUmUfUmUmGmCmUmG*mU*mC |
| 191 | mU*fG*mUmGmAmAmGmCmAmAmUmGmGfUmUfUmUmGmCmUmG*mU*mC |
| 192 | mU*fG*fUmGmAmAfGmCmAmAmUmGmGfUmUfUmUmGmCmUmG*mU*mC |
| 193 | mC*mA*mGmCmAmAAmAfCfCfAmUmUmGmCmUmUmCmA*mC*mA |
| 194 | mU*fG*mUfGmAfAfGmCfAmAmUmGmGfUmUfUmUmGmCmUmG*mU*mC |
| 195 | mU*fG*mUmGfAmAmGmCmAmAmUmGmGfUmUfUmUmGmCmUmG*mU*mC |
| 196 | mU*fG*mUmGfAfAfGfCmAmAmUmGmGfUmUfUmUmGmCmUmG*mU*mC |
| 197 | mC*mA*mGmCmAmAfAmAfCdCfAmUmUmGmCmUmUmCmA*mC*mA |
| 198 | mG*mA*mUmGmCmCmUmGfAfAfCmUmUmGmAmAmUmUmA*mA*mA |
| 199 | mU*fU*mUmAfAmUfUmCmAmAmGmUmUfCmAfGmGmCmAmUmC*mU*mA |
| 200 | mC*mU*mGmUmAmAmCmAfCfAfAmGmUmAmGmAmUmGmC*mC*mA |
| 201 | mU*fG*mGmCfAmUfCmUmAmCmUmUmGfUmGfUmUmAmCmAmG*mC*mA |
| 202 | mC*mC*mUmGmAmUmCmAfCfUfAmUmGmCmAmUmUmUmU*mA*mA |
| 203 | mU*fU*mAmAfAmAfUmGmCmAmUmAmGfUmGfAmUmCmAmGmG*mA*mA |
| 204 | mU*mG*mAmCmAmGmCmUfGfUfGmCmUmGmUmAmAmCmA*mC*mA |
| 205 | mU*fG*mUmGfUmUfAmCmAmGmCmAmCfAmGfCmUmGmUmCmA*mA*mA |
| 206 | mC*mA*mUmGmAmAmUmAfGfAfUmUmCmUmCmUmCmCmU*mG*mA |
| 207 | mU*fC*mAmGfGmAfGmAmGmAmAmUmCfUmAfUmUmCmAmUmG*mC*mA |
| 208 | mG*mU*mAmCmAmGmAmAfUfCfAmUmUmGmCmUmUmAmU*mG*mA |
| 209 | mU*fC*mAmUfAmAfGmCmAmAmUmGmAfUmUfCmUmGmUmAmC*mA*mA |
| 210 | mU*mG*mUmCmCmAmCmGfUfAfUmCmUmUmUmGmGmGmU*mC*mA |
| 211 | mG*mU*mCmCmAmCmGmUfAfUfCmUmUmUmGmGmGmUmC*mU*mA |
| 212 | mG*mA*mUmAmUmAmGmGfAfAfUmUmAmAmGmAmGmGmA*mU*mA |
| 213 | mA*mC*mAmGmAmUmUmGfCfUfGmCmUmUmCmUmGmCmU*mA*mA |
| 214 | UAGUGAAGCAAUGGUUUUGCUG |
| 215 | mU*fA*mGmUfGmAfAmGmCmAmAmUmGfGmUfUmUmUmGmCmU*mG |
| 216 | mU*fA*mGmUfGmAfAmGmCmAmAmUmGfGmUfUmUmUmGmC*mU*mG*mU |
| 217 | mU*fA*mGmUfGmAfAmGmCmAmAmUmGfGmUfUmUmUmGmCmU*mG*mU[iAb] |
| 218 | mU*fA*mGmUfGmAfAmGmCmAmAmUmGfGmUfUmUmUmGmC*mU*mG |
| 219 | mC*mC*mAmAmGmAmUmGfCfAfGmCmAmGmAmAmCmGmG*mC*mA |
| 220 | mU*fG*mCmCfGmUfUmCmUmGmCmUmGfCmAfUmCmUmUmGmG*mA*mC |
| 221 | mA*mG*mGmAmAmGmCmAfGfCfCmAmAmCmGmAmGmAmG*mA*mA |
| 222 | mU*fU*mCmUfCmUfCmGmUmUmGmGmCfUmGfCmUmUmCmCmU*mG*mU |
| 223 | mC*mU*mUmUmGmAmGmCfAfGfAmUmGmCmAmGmAmAmC*mU*mA |
| 224 | mU*fA*mGmUfUmCfUmGmCmAmUmCmUfGmCfUmCmAmAmAmG*mA*mA |
| 225 | mG*mG*mCmAmGmUmUmAfUfCfCmAmGmCmAmUmUmUmC*mC*mA |
| 226 | mU*fG*mGmAfAmAfUmGmCmUmGmGmAfUmAfAmCmUmGmCmC*mU*mU |
| 227 | mG*mA*mCmAmAmAmGmUfAfGfUmAmGmAmAmGmUmAmG*mC*mA |
| 228 | mU*fG*mCmUfAmCfUmUmCmUmAmCmUfAmCfUmUmUmGmUmC*mU*mU |
| 229 | mA*mG*mGmCmAmGmUmUfAfUfCmCmAmGmCmAmUmUmU*mC*mA |
| 230 | mU*fG*mAmAfAmUfGmCmUmGmGmAmUfAmAfCmUmGmCmCmU*mU*mC |
| 231 | mA*mG*mCmAmCmCmGmAfGfAfGmAmGmAmAmUmGmUmC*mC*mA |
| 232 | mU*fG*mGmAfCmAfUmUmCmUmCmUmCfUmCfGmGmUmGmCmU*mU*mG |
| 233 | mA*mA*mCmCmAmGmUmGfAfCfCmAmUmCmCmAmGmAmA*mC*mA |
| 234 | mU*fG*mUmUfCmUfGmGmAmUmGmGmUfCmAfCmUmGmGmUmU*mG*mG |
| 235 | mA*mC*mCmGmAmGmGmAfCfUfGmAmCmCmAmCmUmCmG*mA*mA |
| 236 | mU*fU*mCmGfAmGfUmGmGmUmCmAmGfUmCfCmUmCmGmGmU*mC*mG |
| 237 | mC*mA*mCmAmCmCmGmUfCfGfCmCmAmAmAmGmAmGmA*mC*mA |
| 238 | mU*fG*mUmCfUmCfUmUmUmGmGmCmGfAmCfGmGmUmGmUmG*mC*mC |
| 239 | mG*mC*mCmAmAmGmCmAfCfCfGmAmGmAmGmAmGmAmA*mU*mA |
| 240 | mU*fA*mUmUfCmUfCmUmCmUmCmGmGfUmGfCmUmUmGmGmC*mC*mU |
| 241 | mG*mA*mUmCmCmGmGmUfCfCfCmAmGmGmUmUmAmUmG*mA*mA |
| 242 | mU*fU*mCmAfUmAfAmCmCmUmGmGmGfAmCfCmGmGmAmUmC*mU*mG |

1. An APP RNAi agent comprising Formula (I): (R-L)ₙ-P,
wherein R is a double stranded RNA (dsRNA) comprising a sense stand and an antisense strand, wherein the antisense strand is complementary to APP mRNA;
wherein P is a protein comprising one monovalent human TfR binding domain; and
wherein L is a linker, or optionally absent,
wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6; and
wherein n is an integer of 1 to 3.

2. The APP RNAi agent of claim 1, wherein n is 1.

3. The APP RNAi agent of claim 1, wherein n is 2.

4. The APP RNAi agent of claim 1, wherein VH comprises SEQ ID NO: 7 and VL comprises SEQ ID NO: 8.

5. The APP RNAi agent of claim 1, wherein the human TfR binding domain is a Fab, scFv, Fv, or scFab.

6. The APP RNAi agent of claim 1, wherein the human TfR binding domain further comprises a heavy chain constant region comprising cysteine at residue 124 (according to the EU Index numbering).

7. The APP RNAi agent of claim 1, wherein the human TfR binding domain further comprises a light chain constant region comprising cysteine at residue 156 (according to the EU Index numbering).

8. The APP RNAi agent of claim 1, wherein P further comprises a half-life extender.

9. The APP RNAi agent of claim 8, wherein the half-life extender is an immunoglobulin Fc region or a VHH that binds human serum albumin (HSA).

10. The APP RNAi agent of claim 9, wherein the half-life extender is an immunoglobulin Fc region.

11. The APP RNAi agent of claim 10, wherein the immunoglobulin Fc region is a modified human IgG4 Fc region.

12. The APP RNAi agent of claim 11, wherein the modified human IgG4 Fc region comprises proline at residue 228, and alanine at residues 234 and 235 (all residues are numbered according to the EU Index numbering).

13. The APP RNAi agent of claim 10, wherein P comprises an immunoglobulin Fc region comprising cysteine at residue 378 (according to the EU Index numbering).

14. The APP RNAi agent of claim 10, wherein the immunoglobulin Fc region comprises:
(a) a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering); or
(b) a first Fc CH3 domain comprising leucine at residue 405, and a second Fc CH3 domain comprising arginine at residue 409 (all residues are numbered according to the EU Index numbering).

15. The APP RNAi agent of claim 1, wherein P comprises one heavy chain (HC) and one light chain (LC), wherein HC comprises SEQ ID NO: 9 and LC comprises SEQ ID NO: 10.

16. The APP RNAi agent of claim 1, wherein P comprises two heavy chains HC1 and HC2 and one light chain LC1, wherein HC1 comprises SEQ ID NO: 14, LC1 comprises SEQ ID NO: 10, HC2 comprises SEQ ID NO: 15.

17. The APP RNAi agent of claim 1, wherein P comprises two heavy chains HC1 and HC2 and one light chain LC1, wherein HC1 comprises SEQ ID NO: 16, LC1 comprises SEQ ID NO: 10, HC2 comprises SEQ ID NO: 17.

18. The APP RNAi agent of claim 9, wherein the half-life extender is a VHH that binds HSA.

19. The APP RNAi agent of claim 18, wherein the VHH comprises CDR1 comprising SEQ ID NO: 20, CDR2 comprising SEQ ID NO: 21, and CDR3 comprising SEQ ID NO: 22.

20. The APP RNAi agent of claim 18, wherein the VHH comprises SEQ ID NO: 23.

21. The APP RNAi agent of claim 18, wherein P comprises one heavy chain (HC) and one light chain (LC), and wherein the HC comprises SEQ ID NO: 11 and the LC comprises SEQ ID NO: 12 or 10.

22. The APP RNAi agent of claim 1, wherein P is a heterodimeric antibody that comprises a first arm comprising one monovalent human TfR binding domain and a second arm that is a null arm.

23. The APP RNAi agent of claim 22, wherein the second arm comprises one heavy chain (HC) and one light chain (LC), and wherein the HC comprises SEQ ID NO: 18 and the LC comprises SEQ ID NO: 19.

24. The APP RNAi agent of claim 22, wherein P comprises two heavy chains HC1 and HC2 and two light chains LC1 and LC2, wherein HC1 comprises SEQ ID NO: 13, LC1 comprises SEQ ID NO: 10, HC2 comprises SEQ ID NO: 18, and LC2 comprises SEQ ID NO: 19.

25. The APP RNAi agent of claim 1, wherein, when L is present, L is a Mal-Tet-TCO linker, SMCC linker, or GDM linker.

26. The APP RNAi agent of claim 1, wherein L is a SMCC linker.

27. The APP RNAi agent of claim 1, wherein P is linked to the 3′ end of the sense strand of dsRNA, optionally via the linker.

28. The APP RNAi agent of claim 1, wherein the sense strand and the antisense strand comprise a pair of nucleic acid sequences selected from the group consisting of:
(a) the sense strand comprises SEQ ID NO: 35, and the antisense strand comprises SEQ ID NO: 36;
(b) the sense strand comprises SEQ ID NO: 37, and the antisense strand comprises SEQ ID NO: 38;
(c) the sense strand comprises SEQ ID NO: 39, and the antisense strand comprises SEQ ID NO: 40;
(d) the sense strand comprises SEQ ID NO: 41, and the antisense strand comprises SEQ ID NO: 42;
(e) the sense strand comprises SEQ ID NO: 43, and the antisense strand comprises SEQ ID NO: 44;
(f) the sense strand comprises SEQ ID NO: 45, and the antisense strand comprises SEQ ID NO: 46;
(g) the sense strand comprises SEQ ID NO: 47, and the antisense strand comprises SEQ ID NO: 48;
(h) the sense strand comprises SEQ ID NO: 49, and the antisense strand comprises SEQ ID NO: 50;
(i) the sense strand comprises SEQ ID NO: 51, and the antisense strand comprises SEQ ID NO: 52;
(j) the sense strand comprises SEQ ID NO: 53, and the antisense strand comprises SEQ ID NO: 54;
(k) the sense strand comprises SEQ ID NO: 55, and the antisense strand comprises SEQ ID NO: 56;
(l) the sense strand comprises SEQ ID NO: 57, and the antisense strand comprises SEQ ID NO: 58;
(m) the sense strand comprises SEQ ID NO: 59, and the antisense strand comprises SEQ ID NO: 60;
(n) the sense strand comprises SEQ ID NO: 61, and the antisense strand comprises SEQ ID NO: 62;
(o) the sense strand comprises SEQ ID NO: 63, and the antisense strand comprises SEQ ID NO: 64;
(p) the sense strand comprises SEQ ID NO: 65, and the antisense strand comprises SEQ ID NO: 66;
(q) the sense strand comprises SEQ ID NO: 67, and the antisense strand comprises SEQ ID NO: 68;
(r) the sense strand comprises SEQ ID NO: 69, and the antisense strand comprises SEQ ID NO: 70;
(s) the sense strand comprises SEQ ID NO: 71, and the antisense strand comprises SEQ ID NO: 72;
(t) the sense strand comprises SEQ ID NO: 73, and the antisense strand comprises SEQ ID NO: 74;
(u) the sense strand comprises SEQ ID NO: 75, and the antisense strand comprises SEQ ID NO: 76;
(v) the sense strand comprises SEQ ID NO: 77, and the antisense strand comprises SEQ ID NO: 78;
(w) the sense strand comprises SEQ ID NO: 79, and the antisense strand comprises SEQ ID NO: 80;
(x) the sense strand comprises SEQ ID NO: 81, and the antisense strand comprises SEQ ID NO: 82;
(y) the sense strand comprises SEQ ID NO: 83, and the antisense strand comprises SEQ ID NO: 84;
(z) the sense strand comprises SEQ ID NO: 85, and the antisense strand comprises SEQ ID NO: 86;
(aa) the sense strand comprises SEQ ID NO: 87, and the antisense strand comprises SEQ ID NO: 88;
(bb) the sense strand comprises SEQ ID NO: 89, and the antisense strand comprises SEQ ID NO: 90;
(cc) the sense strand comprises SEQ ID NO: 91, and the antisense strand comprises SEQ ID NO: 92;
(dd) the sense strand comprises SEQ ID NO: 93, and the antisense strand comprises SEQ ID NO: 94;
(ee) the sense strand comprises SEQ ID NO: 95, and the antisense strand comprises SEQ ID NO: 96;
(ff) the sense strand comprises SEQ ID NO: 97, and the antisense strand comprises SEQ ID NO: 98;
(gg) the sense strand comprises SEQ ID NO: 99, and the antisense strand comprises SEQ ID NO: 100;
(hh) the sense strand comprises SEQ ID NO: 184, and the antisense strand comprises SEQ ID NO: 36;
(ii) the sense strand comprises SEQ ID NO: 188, and the antisense strand comprises SEQ ID NO: 38;
(jj) the sense strand comprises SEQ ID NO: 35, and the antisense strand comprises SEQ ID NO: 214;
wherein optionally one or more nucleotides of the sense strand and the antisense strand are independently modified nucleotides, and wherein optionally one or more internucleotide linkages of the sense strand and the antisense strand are modified internucleotide linkages.

29. The APP RNAi agent of claim 28, wherein the sense strand comprises SEQ ID NO: 35, and the antisense strand comprises SEQ ID NO: 36.

30. The APP RNAi agent of claim 28, wherein the sense strand comprises SEQ ID NO: 35, and the antisense strand comprises SEQ ID NO: 214.

31. The APP RNAi agent of claim 28, wherein the sense strand comprises SEQ ID NO: 37, and the antisense strand comprises SEQ ID NO: 38.

32. The APP RNAi agent of claim 1, wherein one or more nucleotides of the sense strand are modified nucleotides.

33. The APP RNAi agent of claim 32, wherein each nucleotide of the sense strand is a modified nucleotide.

34. The APP RNAi agent of claim 1, wherein one or more nucleotides of the antisense strand are modified nucleotides.

35. The APP RNAi agent of claim 34, wherein each nucleotide of the antisense strand is a modified nucleotide.

36. The APP RNAi agent of claim 32, wherein the modified nucleotide is a 2′-fluoro modified nucleotide, 2′-O-methyl modified nucleotide, 2′ deoxy nucleotide (DNA), or 2′-O-alkyl modified nucleotide.

37. The APP RNAi agent of claim 34, wherein the modified nucleotide is a 2′-fluoro modified nucleotide, 2′-O-methyl modified nucleotide, 2′ deoxy nucleotide (DNA), or 2′-O-alkyl modified nucleotide.

38. The APP RNAi agent of claim 36, wherein the sense strand has four 2′-fluoro modified nucleotides at positions 7, 9, 10, and 11 from the 5′ end of the sense strand.

39. The APP RNAi agent of claim 38, wherein nucleotides at positions other than positions 7, 9, 10, and 11 of the sense strand are 2′-O-methyl modified nucleotides.

40. The APP RNAi agent of claim 37, wherein the antisense strand has four 2′-fluoro modified nucleotides at positions 2, 6, 14, and 16 from the 5′ end of the antisense strand.

41. The APP RNAi agent of claim 40, wherein nucleotides at positions other than positions 2, 6, 14 and 16 of the antisense strand are 2′-O-methyl modified nucleotides.

42. The APP RNAi agent of claim 36, wherein the sense strand has three 2′-fluoro modified nucleotides at positions 9, 10, and 11 from the 5′ end of the sense strand.

43. The APP RNAi agent of claim 42, wherein nucleotides at positions other than positions 9, 10, and 11 of the sense strand are 2′-O-methyl modified nucleotides.

44. The APP RNAi agent of claim 37, wherein the antisense strand has five 2′-fluoro modified nucleotides at positions 2, 5, 7, 14, and 16 from the 5′ end of the antisense strand.

45. The APP RNAi agent of claim 44, wherein nucleotides at positions other than positions 2, 5, 7, 14, and 16 of the antisense strand are 2′-O-methyl modified nucleotides.

46. The APP RNAi agent of claim 37, wherein the antisense strand has five 2′-fluoro modified nucleotides at positions 2, 5, 8, 14, and 16 from the 5′ end of the antisense strand.

47. The APP RNAi agent of claim 46, wherein nucleotides at positions other than positions 2, 5, 8, 14, and 16 of the antisense strand are 2′-O-methyl modified nucleotides.

48. The APP RNAi agent of claim 37, wherein the antisense strand has five 2′-fluoro modified nucleotides at positions 2, 3, 7, 14, and 16 from the 5′ end of the antisense strand.

49. The APP RNAi agent of claim 48, wherein nucleotides at positions other than positions 2, 3, 7, 14, and 16 of the antisense strand are 2′-O-methyl modified nucleotides.

50. The APP RNAi agent of claim 1, wherein the sense strand and the antisense strand have one or more modified internucleotide linkages.

51. The APP RNAi agent of claim 50, wherein the modified internucleotide linkage is phosphorothioate linkage.

52. The APP RNAi agent of claim 50, wherein the sense strand has four or five phosphorothioate linkages.

53. The APP RNAi agent of claim 50, wherein the antisense strand has four or five phosphorothioate linkages.

54. The APP RNAi agent of claim 1, wherein the antisense strand has a phosphate analog at the 5′ end.

55. The APP RNAi agent of claim 54, wherein the phosphate analog is 5′-vinylphosphonate.

56. The APP RNAi agent of claim 1, wherein the sense strand or the antisense strand comprises an abasic moiety or inverted abasic moiety.

57. The APP RNAi agent of claim 1, wherein the sense strand and the antisense strand comprise a pair of nucleic acid sequences selected from the group consisting of:
(a) the sense strand comprises SEQ ID NO: 101, and the antisense strand comprises SEQ ID NO: 102, 173, 176, 177, 178, 179, 180, 182, or 185;
(b) the sense strand comprises SEQ ID NO: 103, and the antisense strand comprises SEQ ID NO: 104, 175, 189, 190, 191, 192, 194, 195, or 196;
(c) the sense strand comprises SEQ ID NO: 105, and the antisense strand comprises SEQ ID NO: 106;
(d) the sense strand comprises SEQ ID NO: 107, and the antisense strand comprises SEQ ID NO: 108;
(e) the sense strand comprises SEQ ID NO: 109, and the antisense strand comprises SEQ ID NO: 110;
(f) the sense strand comprises SEQ ID NO: 111, and the antisense strand comprises SEQ ID NO: 112;
(g) the sense strand comprises SEQ ID NO: 113, and the antisense strand comprises SEQ ID NO: 114;
(h) the sense strand comprises SEQ ID NO: 115, and the antisense strand comprises SEQ ID NO: 116;
(i) the sense strand comprises SEQ ID NO: 117, and the antisense strand comprises SEQ ID NO: 118;
(j) the sense strand comprises SEQ ID NO: 119, and the antisense strand comprises SEQ ID NO: 120;
(k) the sense strand comprises SEQ ID NO: 121, and the antisense strand comprises SEQ ID NO: 122;
(l) the sense strand comprises SEQ ID NO: 123, and the antisense strand comprises SEQ ID NO: 124;
(m) the sense strand comprises SEQ ID NO: 125, and the antisense strand comprises SEQ ID NO: 126;
(n) the sense strand comprises SEQ ID NO: 127, and the antisense strand comprises SEQ ID NO: 128;
(o) the sense strand comprises SEQ ID NO: 129, and the antisense strand comprises SEQ ID NO: 130;
(p) the sense strand comprises SEQ ID NO: 131, and the antisense strand comprises SEQ ID NO: 132;
(q) the sense strand comprises SEQ ID NO: 133, and the antisense strand comprises SEQ ID NO: 134;
(r) the sense strand comprises SEQ ID NO: 135, and the antisense strand comprises SEQ ID NO: 136;
(s) the sense strand comprises SEQ ID NO: 137, and the antisense strand comprises SEQ ID NO: 138;
(t) the sense strand comprises SEQ ID NO: 139, and the antisense strand comprises SEQ ID NO: 140;
(u) the sense strand comprises SEQ ID NO: 141, and the antisense strand comprises SEQ ID NO: 142;
(v) the sense strand comprises SEQ ID NO: 143, and the antisense strand comprises SEQ ID NO: 144;
(w) the sense strand comprises SEQ ID NO: 145, and the antisense strand comprises SEQ ID NO: 146;
(x) the sense strand comprises SEQ ID NO: 147, and the antisense strand comprises SEQ ID NO: 148;
(y) the sense strand comprises SEQ ID NO: 149, and the antisense strand comprises SEQ ID NO: 150;
(z) the sense strand comprises SEQ ID NO: 151, and the antisense strand comprises SEQ ID NO: 152;
(aa) the sense strand comprises SEQ ID NO: 153, and the antisense strand comprises SEQ ID NO: 154;
(bb) the sense strand comprises SEQ ID NO: 155, and the antisense strand comprises SEQ ID NO: 156;
(cc) the sense strand comprises SEQ ID NO: 157, and the antisense strand comprises SEQ ID NO: 158;
(dd) the sense strand comprises SEQ ID NO: 159, and the antisense strand comprises SEQ ID NO: 160;
(ee) the sense strand comprises SEQ ID NO: 161, and the antisense strand comprises SEQ ID NO: 162;
(ff) the sense strand comprises SEQ ID NO: 163, and the antisense strand comprises SEQ ID NO: 164;
(gg) the sense strand comprises SEQ ID NO: 165, and the antisense strand comprises SEQ ID NO: 166;
(hh) the sense strand comprises SEQ ID NO: 172, and the antisense strand comprises SEQ ID NO: 173;
(ii) the sense strand comprises SEQ ID NO: 181, and the antisense strand comprises SEQ ID NO: 173;
(jj) the sense strand comprises SEQ ID NO: 174 or 193, and the antisense strand comprises SEQ ID NO: 175;
(kk) the sense strand comprises SEQ ID NO: 172, and the antisense strand comprises SEQ ID NO: 177;
(ll) the sense strand comprises SEQ ID NO: 181, 183, or 186, and the antisense strand comprises SEQ ID NO: 102;
(mm) the sense strand comprises SEQ ID NO: 187, 193, or 197, and the antisense strand comprises SEQ ID NO: 104;
(nn) the sense strand comprises SEQ ID NO: 198, and the antisense strand comprises SEQ ID NO: 199;
(oo) the sense strand comprises SEQ ID NO: 200, and the antisense strand comprises SEQ ID NO: 201;
(pp) the sense strand comprises SEQ ID NO: 202, and the antisense strand comprises SEQ ID NO: 203;
(qq) the sense strand comprises SEQ ID NO: 204, and the antisense strand comprises SEQ ID NO: 205;
(rr) the sense strand comprises SEQ ID NO: 206, and the antisense strand comprises SEQ ID NO: 207;
(ss) the sense strand comprises SEQ ID NO: 208, and the antisense strand comprises SEQ ID NO: 209;
(tt) the sense strand comprises SEQ ID NO: 210, and the antisense strand comprises SEQ ID NO: 118;
(uu) the sense strand comprises SEQ ID NO: 211, and the antisense strand comprises SEQ ID NO: 120;
(vv) the sense strand comprises SEQ ID NO: 212, and the antisense strand comprises SEQ ID NO: 122;
(ww) the sense strand comprises SEQ ID NO: 213, and the antisense strand comprises SEQ ID NO: 124;
(xx) the sense strand comprises SEQ ID NO: 172, and the antisense strand comprises SEQ ID NO: 215;
(yy) the sense strand comprises SEQ ID NO: 172, and the antisense strand comprises SEQ ID NO: 216;
(zz) the sense strand comprises SEQ ID NO: 172, and the antisense strand comprises SEQ ID NO: 217;
(aaa) the sense strand comprises SEQ ID NO: 172, and the antisense strand comprises SEQ ID NO: 218;
(bbb) the sense strand comprises SEQ ID NO: 219, and the antisense strand comprises SEQ ID NO: 220;
(ccc) the sense strand comprises SEQ ID NO: 221, and the antisense strand comprises SEQ ID NO: 222;
(ddd) the sense strand comprises SEQ ID NO: 223, and the antisense strand comprises SEQ ID NO: 224;
(eee) the sense strand comprises SEQ ID NO: 225, and the antisense strand comprises SEQ ID NO: 226;
(fff) the sense strand comprises SEQ ID NO: 227, and the antisense strand comprises SEQ ID NO: 228;
(ggg) the sense strand comprises SEQ ID NO: 229, and the antisense strand comprises SEQ ID NO: 230;
(hhh) the sense strand comprises SEQ ID NO: 231, and the antisense strand comprises SEQ ID NO: 232;
(iii) the sense strand comprises SEQ ID NO: 233, and the antisense strand comprises SEQ ID NO: 234;
(jjj) the sense strand comprises SEQ ID NO: 235, and the antisense strand comprises SEQ ID NO: 236;
(kkk) the sense strand comprises SEQ ID NO: 237, and the antisense strand comprises SEQ ID NO: 238;
(lll) the sense strand comprises SEQ ID NO: 239, and the antisense strand comprises SEQ ID NO: 240; and
(mmm) the sense strand comprises SEQ ID NO: 241, and the antisense strand comprises SEQ ID NO: 242.

58. The APP RNAi agent of claim 1, wherein the sense strand and the antisense strand have a pair of nucleic acid sequences selected from the group consisting of:
(a) the sense strand consists of SEQ ID NO: 101, and the antisense strand consists of SEQ ID NO: 102, 173, 176, 177, 178, 179, 180, 182, or 185;
(b) the sense strand consists of SEQ ID NO: 103, and the antisense strand consists of SEQ ID NO: 104, 175, 189, 190, 191, 192, 194, 195, or 196;
(c) the sense strand consists of SEQ ID NO: 105, and the antisense strand consists of SEQ ID NO: 106;
(d) the sense strand consists of SEQ ID NO: 107, and the antisense strand consists of SEQ ID NO: 108;
(e) the sense strand consists of SEQ ID NO: 109, and the antisense strand consists of SEQ ID NO: 110;
(f) the sense strand consists of SEQ ID NO: 111, and the antisense strand consists of SEQ ID NO: 112;
(g) the sense strand consists of SEQ ID NO: 113, and the antisense strand consists of SEQ ID NO: 114;
(h) the sense strand consists of SEQ ID NO: 115, and the antisense strand consists of SEQ ID NO: 116;
(i) the sense strand consists of SEQ ID NO: 117, and the antisense strand consists of SEQ ID NO: 118;
(j) the sense strand consists of SEQ ID NO: 119, and the antisense strand consists of SEQ ID NO: 120;
(k) the sense strand consists of SEQ ID NO: 121, and the antisense strand consists of SEQ ID NO: 122;
(l) the sense strand consists of SEQ ID NO: 123, and the antisense strand consists of SEQ ID NO: 124;
(m) the sense strand consists of SEQ ID NO: 125, and the antisense strand consists of SEQ ID NO: 126;
(n) the sense strand consists of SEQ ID NO: 127, and the antisense strand consists of SEQ ID NO: 128;
(o) the sense strand consists of SEQ ID NO: 129, and the antisense strand consists of SEQ ID NO: 130;
(p) the sense strand consists of SEQ ID NO: 131, and the antisense strand consists of SEQ ID NO: 132;
(q) the sense strand consists of SEQ ID NO: 133, and the antisense strand consists of SEQ ID NO: 134;
(r) the sense strand consists of SEQ ID NO: 135, and the antisense strand consists of SEQ ID NO: 136;
(s) the sense strand consists of SEQ ID NO: 137, and the antisense strand consists of SEQ ID NO: 138;
(t) the sense strand consists of SEQ ID NO: 139, and the antisense strand consists of SEQ ID NO: 140;
(u) the sense strand consists of SEQ ID NO: 141, and the antisense strand consists of SEQ ID NO: 142;
(v) the sense strand consists of SEQ ID NO: 143, and the antisense strand consists of SEQ ID NO: 144;
(w) the sense strand consists of SEQ ID NO: 145, and the antisense strand consists of SEQ ID NO: 146;
(x) the sense strand consists of SEQ ID NO: 147, and the antisense strand consists of SEQ ID NO: 148;
(y) the sense strand consists of SEQ ID NO: 149, and the antisense strand consists of SEQ ID NO: 150;
(z) the sense strand consists of SEQ ID NO: 151, and the antisense strand consists of SEQ ID NO: 152;
(aa) the sense strand consists of SEQ ID NO: 153, and the antisense strand consists of SEQ ID NO: 154;
(bb) the sense strand consists of SEQ ID NO: 155, and the antisense strand consists of SEQ ID NO: 156;
(cc) the sense strand consists of SEQ ID NO: 157, and the antisense strand consists of SEQ ID NO: 158;
(dd) the sense strand consists of SEQ ID NO: 159, and the antisense strand consists of SEQ ID NO: 160;
(ee) the sense strand consists of SEQ ID NO: 161, and the antisense strand consists of SEQ ID NO: 162;
(ff) the sense strand consists of SEQ ID NO: 163, and the antisense strand consists of SEQ ID NO: 164;
(gg) the sense strand consists of SEQ ID NO: 165, and the antisense strand consists of SEQ ID NO: 166, (hh) the sense strand consists of SEQ ID NO: 172, and the antisense strand consists of SEQ ID NO: 173;
(ii) the sense strand consists of SEQ ID NO: 181, and the antisense strand consists of SEQ ID NO: 173;
(jj) the sense strand consists of SEQ ID NO: 174 or 193, and the antisense strand consists of SEQ ID NO: 175;
(kk) the sense strand consists of SEQ ID NO: 172, and the antisense strand consists of SEQ ID NO: 177;
(ll) the sense strand consists of SEQ ID NO: 181, 183, or 186, and the antisense strand consists of SEQ ID NO: 102;
(mm) the sense strand consists of SEQ ID NO: 187, 193, or 197, and the antisense strand consists of SEQ ID NO: 104;
(nn) the sense strand consists of SEQ ID NO: 198, and the antisense strand consists of SEQ ID NO: 199;
(oo) the sense strand consists of SEQ ID NO: 200, and the antisense strand consists of SEQ ID NO: 201;
(pp) the sense strand consists of SEQ ID NO: 202, and the antisense strand consists of SEQ ID NO: 203;
(qq) the sense strand consists of SEQ ID NO: 204, and the antisense strand consists of SEQ ID NO: 205;
(rr) the sense strand consists of SEQ ID NO: 206, and the antisense strand consists of SEQ ID NO: 207;
(ss) the sense strand consists of SEQ ID NO: 208, and the antisense strand consists of SEQ ID NO: 209;
(tt) the sense strand consists of SEQ ID NO: 210, and the antisense strand consists of SEQ ID NO: 118;
(uu) the sense strand consists of SEQ ID NO: 211, and the antisense strand consists of SEQ ID NO: 120;
(vv) the sense strand consists of SEQ ID NO: 212, and the antisense strand consists of SEQ ID NO: 122;
(ww) the sense strand consists of SEQ ID NO: 213, and the antisense strand consists of SEQ ID NO: 124;
(xx) the sense strand consists of SEQ ID NO: 172, and the antisense strand consists of SEQ ID NO: 215;
(yy) the sense strand consists of SEQ ID NO: 172, and the antisense strand consists of SEQ ID NO: 216;
(zz) the sense strand consists of SEQ ID NO: 172, and the antisense strand consists of SEQ ID NO: 217;
(aaa) the sense strand consists of SEQ ID NO: 172, and the antisense strand consists of SEQ ID NO: 218;
(bbb) the sense strand consists of SEQ ID NO: 219, and the antisense strand consists of SEQ ID NO: 220;
(ccc) the sense strand consists of SEQ ID NO: 221, and the antisense strand consists of SEQ ID NO: 222;
(ddd) the sense strand consists of SEQ ID NO: 223, and the antisense strand consists of SEQ ID NO: 224;
(eee) the sense strand consists of SEQ ID NO: 225, and the antisense strand consists of SEQ ID NO: 226;
(fff) the sense strand consists of SEQ ID NO: 227, and the antisense strand consists of SEQ ID NO: 228;
(ggg) the sense strand consists of SEQ ID NO: 229, and the antisense strand consists of SEQ ID NO: 230;
(hhh) the sense strand consists of SEQ ID NO: 231, and the antisense strand consists of SEQ ID NO: 232;
(iii) the sense strand consists of SEQ ID NO: 233, and the antisense strand consists of SEQ ID NO: 234;
(jjj) the sense strand consists of SEQ ID NO: 235, and the antisense strand consists of SEQ ID NO: 236;
(kkk) the sense strand consists of SEQ ID NO: 237, and the antisense strand consists of SEQ ID NO: 238;
(lll) the sense strand consists of SEQ ID NO: 239, and the antisense strand consists of SEQ ID NO: 240; and
(mmm) the sense strand consists of SEQ ID NO: 241, and the antisense strand consists of SEQ ID NO: 242.

59. A pharmaceutical composition comprising the APP RNAi agent of claim 1 and a pharmaceutically acceptable carrier.

60. A method of treating an APP associated neurological disease in a patient in need thereof, the method comprising administering to the patient an effective amount of the APP RNAi agent of claim 1.

61. The method of claim 60, wherein the APP associated neurological disease is selected from Alzheimer's disease, Down's syndrome, or cerebral amyloid angiopathy.

62. The method of claim 60, wherein the APP RNAi agent is administered to the patient intravenously or subcutaneously.
